(19) United States
(12) Patent Application Publication
(10) Pub. No.: US 2025/0179012 A1
(43) Pub. Date: Jun. 5, 2025

(54) SYNTHESIS OF SULFUR(VI) COMPOUNDS AND REAGENTS FOR SAME

(71) Applicant: H. LEE MOFFITT CANCER CENTER AND RESEARCH INSTITUTE, INC., Tampa, FL (US)

(72) Inventors: Justin Matthew LOPCHUK, Tampa, FL (US); Zachary SHULTZ, Tampa, FL (US)

(21) Appl. No.: 18/843,828

(22) PCT Filed: Mar. 6, 2023
(86) PCT No.: PCT/US2023/014592

§ 371 Date: Sep. 4, 2024

Related U.S. Application Data
(60) Provisional application No. 63/316,676, filed on Mar. 4, 2022.

(51) Int. Cl. C07C 311/02; C07C 303/40

(52) U.S. Cl. CPC C07C 311/02; C07C 303/40

(57) ABSTRACT

This disclosure provides reagents and processes for the synthesis of organic compounds, more particularly reagents and processes for the asymmetric synthesis of sulfur (VI) containing organic compounds.

### CROSS-REFERENCE TO RELATED APPLICATIONS

This application claims the benefit of priority to U.S. Provisional Application No. 63/316,676, filed Mar. 4, 2022, the disclosure of which is incorporated herein by reference in its entirety.

### STATEMENT REGARDING FEDERALLY SPONSORED RESEARCH OR DEVELOPMENT

This invention was made with government support under grant number R35GM142577 awarded by the National Institutes of Health. The Government has certain rights in the invention.

### TECHNICAL FIELD

This disclosure relates to reagents and processes for the synthesis of organic compounds, and more particularly to reagents and processes for the asymmetric synthesis of sulfur (VI) containing organic compounds.

### BACKGROUND

The ability of sulfur to adopt a range of oxidation states (II-VI) with defined molecular geometries has led to many advancements in the discovery sciences (see Ilardi, E. A. et al. J. Med. Chem. 2014, 57 (7), 2832-2842; Zhao, C.; et al. Eur. J. Med. Chem. 2019, 162, 679-734; Tang, K.-X. et al. Adv. Synth. Catal. 2019, 361 (1), 26-38; and Wang, J. et al. Nat. Mater. 2021, 20 (5), 665-673). From materials to medicines, sulfur-containing functional groups are pervasive across disciplines. The more common S(VI) functional groups, such as sulfones and sulfonamides, have attracted the most attention-finding their way into many drug discovery programs and greater than 70 FDA approved drugs (see Scott, K. A. et al. Top Curr Chem. 2018, 376, 5). More recently, the exploration of historically neglected sulfonimidoyl S(VI) functional groups, containing S═N and S—N bond(s), has provided novel clinical candidates for a variety of indications as well as important agrochemicals (see Mäder, P. et al. J. Med. Chem. 2020, 63 (23), 14243-14275; Lücking, U. Angew. Chem. Int. Ed. 2013, 52 (36), 9399-9408; Sirvent, J. A. et al. ChemMedChem 2017, 12 (7), 487-501; Lücking, U. Org. Chem. Front. 2019, 6 (8), 1319-1324; Devendar, P. et al. Top Curr Chem. 2017, 375 (6), 82; Zasukha, S. V. et al. Chem.—Eur. J. 2019, 25 (28), 6928-6940; and Chinthakindi, P. K. et al. Angew. Chem. Int. Ed. 2017, 56 (15), 4100-4109).

Sulfoximines, the mono-aza S═N variants of sulfones, have recently been accepted in medicinal chemistry as bioisosteres or viable replacement groups for carboxylic acids, alcohols sulfones and sulfonamides (see Foote, K. M. et al. J. Med. Chem. 2018, 61 (22), 9889-9907; WO 2017/060167 A1; WO 2017/055196 A1; Zhu, Y. et al. J. Agric. Food Chem. 2011, 59 (7), 2950-2957; WO 2014/172443 A1; Lücking, U. et al. J. Med. Chem. 2021, 64 (15), 11651-11674; and Frings, M. et al. Eur. J. Med. Chem. 2017, 126, 225-245). Additionally, sulfonimidamides can serve as bioisosteres for amines, sulfones and sulfonamides. The unique H-bond donor and acceptor properties of the sulfonimidoyl groups allow them to mimic a wide range of other functionality while commonly providing other advantages, such as a chiral environment and increases in aqueous solubility (see Lücking, U. et al. ChemMedChem 2013, 8 (7), 1067-1085). Recent enthusiasm over the physiochemical properties of sulfoximines (and other sul-fonimidoyl groups) has led to an exponential increase in their use to improve pharmacokinetic (PK) and pharmacodynamic (PD) properties during lead optimization studies (see Izzo, F. et al. Chem.-Eur. J. 2018, 24 (37), 9295-9304).

A specific example of this was demonstrated by AstraZeneca during the discovery and subsequent development of their ATR inhibitor, ceralasertib. Amid the final optimization stage of the drug discovery program, a sulfone was replaced for a sulfoximine. The resulting introduction of a sulfoximine led to an increased aqueous solubility while maintaining potency, which allowed for the advancement of ceralasertib to the clinic where it is currently undergoing multiple phase II clinical trials (see Clinical Trials Using ATR Kinase Inhibitor AZD6738. https://www.cancer.gov/about-cancer/treatment/clinical-trials/intervention/atr-kinase-inhibitor-azd6738, accessed May 26, 2021). Other discovery programs at Bayer, Pfizer, Genentech, Hoffman-La Roche, Novartis, Nestlé Skin Health and Corteva Agriscience, have been actively researching neglected S(VI) functional groups with respect to methods for their installation and incorporation into lead scaffolds (see WO 2018/177899 A1; Walker, D. P. et al. Bioorg. Med. Chem. Lett. 2009, 19 (12), 3253-8; WO 2020/018975 A1; WO 2017/172802 A1; WO 2016/012422 A1; WO 2020/154499 A1; Ouvry, G. et al. Bioorg. Med. Chem. Lett. 2018, 28 (8), 1269-1273; and Loso, M. R. et al. Bioorg. Med. Chem. 2016, 24 (3), 378-382).

Furthermore, the novelty of sulfonimidoyl groups, with their inherent stereochemical and additional spatial vectors capable of modifications, provides ample opportunities for new intellectual property (IP) development. An increase in patent applications and issuances within the last ten years is a growing testament to the untapped potential of sulfoximines (2,074 total patents since their first report in 1953, 1,536 of those coming in the last decade), sulfonimidamides (140 total patents since their first report in 1967, 121 of those coming in the last decade), sulfondiimines (33 total patents since their first report in 1967, 18 of those coming in the last decade) and related functional groups. Pioneering work by Bolm, Bull, Johnson, Luecking, Maruoka, Sharpless, Willis and others for the creation and modifications of S(VI) functionality has given rise to new possibilities in the field. However, despite the increase in methods to access neglected sulfonimidoyl-containing compounds, there has been relatively little advancement toward the modular installation of these groups to pharmaceutical scaffolds (see SciFinder; Chemical Abstracts Service: Columbus, OH; Patent search for sulfoximine-containing compounds: structure search using general sulfoximine; https://scifinder-n.cas.org/search/reference/60b0d9dde91ebe7b813ae2f8/1 (accessed May 28); SciFinder; Chemical Abstracts Service: Columbus, OH; Patent search for sulfonimidamide-containing compounds: structure search using general sulfonimidamide; https://scifinder-n.cas.org/search/reference/60b0da8be91ebe7b813ae3af/1 (accessed May 28); SciFinder; Chemical Abstracts Service: Columbus, OH; Patent search for sulfondiimine-containing compounds: structure search using general sulfondiimine; https://scifinder-n.cas.org/search/reference/60b0dae9e91ebe7b813ae41a/1 (accessed May 28); Okamura, H. et al. Org. Lett. 2004, 6 (8), 1305-1307; Correa, A. et al. Adv. Synth. Catal. 2007, 349 (17+18), 2673-2676; Cheng, Y.; Bolm, C., Regioselective Syntheses of 1,2-Benzothiazines by Rhodium-Catalyzed Annulation Reactions. Angew. Chem. Int. Ed. 2015, 54 (42), 12349-12352; Zenzola, M. et al. J. Org. Chem. 2015, 80 (12), 6391-6399; Zenzola, M. et al. Angew. Chem., Int. Ed. 2016, 55 (25), 7203-7207; Tota, A. et al. Chem. Commun. 2017, 53 (2), 348-351; Johnson, C. R. Acc. Chem. Res. 1973, 6 (10), 341-347; Sirvent, J. A. et al. Synthesis 2017, 49 (05), 1024-1036; Greed, S. et al. Chem.-Eur. J. 2020, 26 (55), 12533-12538; Izzo, F. et al. Chem.-Eur. J. 2017, 23 (60), 15189-15193; Aota, Y. et al. Angew. Chem. Int. Ed. 2019, 58 (49), 17661-17665; Aota, Y. et al. J. Am. Chem. Soc. 2019, 141 (49), 19263-19268; Gao, B. et al. Angew. Chem., Int. Ed. 2018, 57 (7), 1939-1943; Zheng, Q. et al. Proc. Natl. Acad. Sci. U.S.A. 2019, 116 (38), 18808; Kitamura, S. et al. J. Am. Chem. Soc. 2020, 142 (25), 10899-10904; Davies, T. Q. et al. Angew. Chem. Int. Ed. 2017, 56 (47), 14937-14941; Zhang, Z.-X. et al. J. Am. Chem. Soc. 2019, 141 (33), 13022-13027; Davies, T. Q. et al. J. Am. Chem. Soc. 2020, 142 (36), 15445-15453; Pyne, S. G. et al. J. Org. Chem. 1997, 62 (8), 2337-2343; Zhang, W. et al. Org. Lett. 2009, 11 (10), 2109-2112; and Shen, X. et al. Eur. J. Org. Chem. 2016, 2016 (5), 906-909).

Owing to the growing attention of higher order sulfur-based functional groups as bioisosteres and PK modulators in the pharmaceutical sciences, the unmet need for their incorporation into medicinally relevant structures with an emphasis on asymmetric control must be addressed.

There is a clear need for new methods and reagents for the synthesis of sulfur (VI) compounds. The present disclosure addresses these as well as other needs.

### SUMMARY

In accordance with the purposes of the disclosed materials and methods, as embodied and broadly defined herein, the disclosed subject matter, in one aspect, related to processes for the synthesis of compounds as well as compounds made by said processes.

In one aspect, a process is provided for synthesis of a compound of Formula I

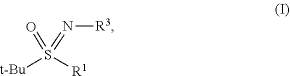

the process comprising:
reacting a compound of Formula II

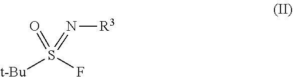

with a compound of Formula III

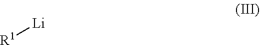

to form the compound of Formula I;
wherein all variables are as defined herein.

In another aspect, a compound of Formula I prepared according to the processes described herein is also provided.

In another aspect, a compound of Formula II is provided:

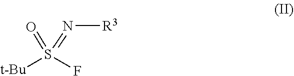

wherein all variables are as defined herein.

In another aspect, a process is provided for synthesis of a compound of Formula V

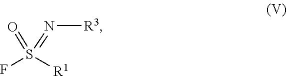

the process comprising:
(a) reacting a compound of Formula II

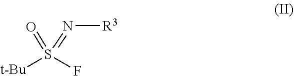

with a compound of Formula III

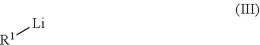

to form a compound of Formula I

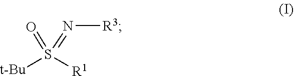

(b) reacting the compound of Formula I with a first base to form a compound of Formula IV

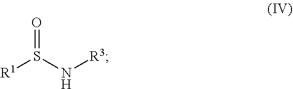

and
(c) reacting the compound of Formula IV with a fluorinating reagent and a second base to form the compound of Formula V;
Wherein all variables are as defined herein.

In another aspect, a compound of Formula V prepared according to the processes described herein is also provided.

In another aspect, a process is provided for synthesis of a compound of Formula VII

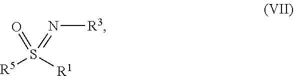

the process comprising:
(a) reacting a compound of Formula II

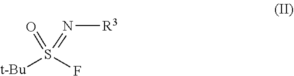

with a compound of Formula III

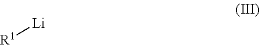

to form a compound of Formula I

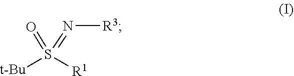

(b) reacting the compound of Formula I with a first base to form a compound of Formula IV

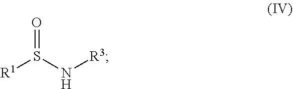

(c) reacting the compound of Formula IV with a fluorinating reagent and a second base to form a compound of Formula V

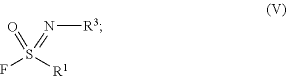

and
(d) reacting the compound of Formula V with a compound of Formula VI

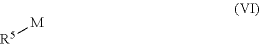

to form the compound of Formula VII;
wherein all variables are as defined herein.

In another aspect, a compound of Formula VII is provided prepared according to the processes described herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a base”, or “a solvent”, includes, but is not limited to, two or more such compounds, bases, solvents, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl (C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or goups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

“Protecting group”, as used herein, refers to any convenient functional group that allows to obtain chemoselectivity in a subsequent chemical reaction. Protecting groups are described, for example, in Greene & Wuts, eds., “Protecting Groups in Organic Synthesis”, 2nd ed. New York; John Wiley & Sons, Inc., 1991. For a particular compound and/or a particular chemical reaction, a person skilled in the art knows how to select and implement appropriate protecting groups and synthetic methods. Examples of amine protecting groups include acyl and alkoxycarbonyl groups, such as t-butoxycarbonyl (BOC), and [2-(trimethylsilyl) ethoxy]methyl (SEM). Examples of carboxyl protecting groups include (1-6C) alkyl groups, such as methyl, ethyl and t-butyl. Examples of alcohol protecting groups include benzyl, trityl, silyl ethers, and the like.

As used herein, the term “composition” is intended to encompass a product comprising the specified ingredients in the specified amounts, as well as any product which results, directly or indirectly, from combination of the specified ingredients in the specified amounts.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Compounds of Formula II

In one aspect, a compound is provided of Formula II:

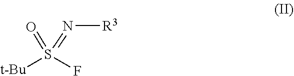

wherein R³ is —C(═O)—N(R⁴)₂, and wherein R⁴ is independently selected at each occurrence from C₃-C₅ alkyl. In some aspects of Formula II, each R⁴ is isopropyl.

In some aspects, the compound of Formula II is selected from

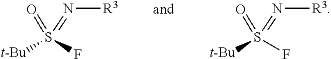

In another aspect, a compound is of Formula II-a

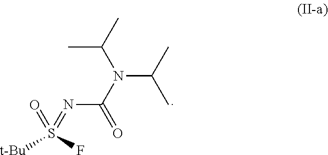 [SMILES: CC(C)N(C(=O)N=[S@](=O)(F)C(C)(C)C)C(C)C]

In another aspect, a compound is of Formula II-b

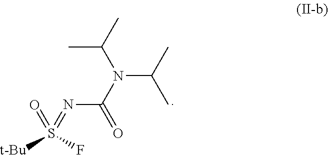 [SMILES: CC(C)N(C(=O)N=[S@@](=O)(F)C(C)(C)C)C(C)C]

### Processes for Synthesis of Compounds of Formula I

In another aspect, a process is provided for synthesis of a compound of Formula I

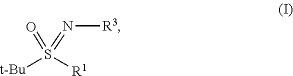

the process comprising:
reacting a compound of Formula II

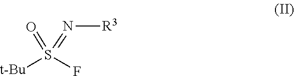

with a compound of Formula III

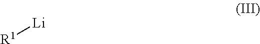

to form the compound of Formula I;
wherein:
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴)₂, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects, each R⁴ is independently selected from isopropyl, sec-butyl, tert-butyl, and isopentyl. In some aspects, each R⁴ is isopropyl.

In some aspects, the compound of Formula II is selected from:

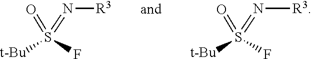

In some embodiments, the process described herein may further be run in the presence of a solvent. The solvent may typically comprise one or more solvents which are unreactive with the selection of compounds used in the process. In some aspects, the solvent comprises an ethereal solvent, for example diethyl ether, tetrahydrofuran, 1,4-dioxane, cyclopentyl methyl ether, or combinations thereof. Other suitable solvents may be used as would be apparent to a person of skill in the art. In some aspects, the process is performed at a temperature from about-80 degrees Celsius to about-40 degrees Celsius.

In another aspect, a compound of Formula I prepared according to the processes described herein is also provided.

### Processes for Synthesis of Compounds of Formula V

In another aspect, a process is provided for synthesis of a compound of Formula V

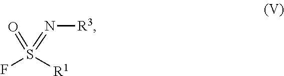

the process comprising:
(a) preparing a compound of Formula I according to the process described herein;
(b) reacting the compound of Formula I with a first base to form a compound of Formula IV

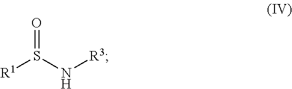

and
(c) reacting the compound of Formula IV with a fluorinating reagent and a second base to form the compound of Formula V

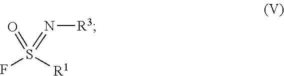

wherein all variables are as defined herein.

In some aspects, the first base comprises a metal alkoxide. In some aspects, the first base comprises a metal tert-butoxide, such as sodium or potassium tert-butoxide.

In some aspects, step (b) is performed in the presence of a solvent. The solvent may typically comprise one or more solvents which is unreactive with the selection of compounds used in step (b). In some aspects, the solvent comprises an ethereal solvent, for example diethyl ether, tetrahydrofuran, 1,4-dioxane, cyclopentyl methyl ether, or combinations thereof. In some aspects, step (b) is performed at a temperature from about 25 degrees Celsius to about 100 degrees Celsius.

In some aspects, the fluorinating agent comprises an electrophilic fluorinating agent. In some aspects, the fluorinating agent comprises N-fluorobenzenesulfonimide. In some aspects, the second base comprises a metal hydride. In some aspects, the second base comprises sodium hydride, potassium hydride, or cesium hydride.

In some aspects, step (c) is performed in the presence of a solvent. The solvent may typically comprise one or more solvents which are unreactive with the selection of compounds used in step (c). In some aspects, the solvent comprises an ethereal solvent, for example diethyl ether, tetrahydrofuran, 1,4-dioxane, cyclopentyl methyl ether, or combinations thereof. Other suitable solvents may be used as would be apparent to a person of skill in the art. In some aspects, step (c) is performed at a temperature ranging from about −40 degrees Celsius to about 30 degrees Celsius.

In another aspect, a process is provided for synthesis of a compound of Formula V

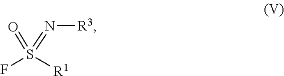

the process comprising:
(a) preparing a compound of Formula I according to the processes described herein;
(b) reacting the compound of Formula I with a first base to form a reaction mixture;
(c) reacting the first reaction mixture with a fluorinating reagent in the presence of an acid to form the compound of Formula V

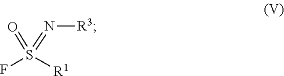

wherein all variables are as defined herein.

In some aspects, the first base comprises a metal alkoxide. In some aspects, the first base comprises a metal tert-butoxide, such as sodium or potassium tert-butoxide. In some aspects, step (b) is performed at a temperature from about 25 degrees Celsius to about 100 degrees Celsius. In some aspects, step (b) is performed in the presence of a solvent. The solvent may typically comprise one or more solvents which are unreactive with the selection of compounds used in step (b). In some aspects, the solvent comprises an ethereal solvent, for example diethyl ether, tetrahydrofuran, 1,4-dioxane, cyclopentyl methyl ether, or combinations thereof. Other suitable solvents may be used as would be apparent to a person of skill in the art. In some aspects, the reaction mixture formed in step (b) is not isolated or purified before performing step (c).

In some aspects, the fluorinating agent comprises an electrophilic fluorinating agent. In some aspects, the fluorinating agent comprises N-fluorobenzenesulfonimide. In some aspects, the acid comprises an organic acid, for example acetic acid. In some aspects, step (c) is performed at a temperature ranging from about-40 degrees Celsius to about 30 degrees Celsius.

In another aspect, a compound of Formula V prepared according to the processes described herein.

### Processes for Synthesis of Compounds of Formula VII and Formula VII-a

In another aspect, a process is provided for synthesis of a compound of Formula VII

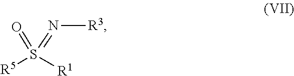

the process comprising:
(a) preparing a compound of Formula V according to the processes described herein;
(b) reacting the compound of Formula V with a compound of Formula VI

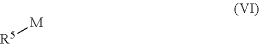

to form the compound of Formula VII;
wherein:
R⁵ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, and (R^{x}R^{y}N)—(C₀-C₃ alkyl)-, wherein each R⁵ is optionally substituted with one or more Z groups as allowed by valency;
M is selected from Li, Na, MgX¹, and MgX¹LiX², wherein X¹ and X² are each independently halo; and
all other variables are as defined herein.

In some aspects, M is Li. In some aspects, M is Na. In some aspects, M is MgCl or MgBr. In some aspects, M is MgBrLiCl or MgClLiCl.

In some aspects, step (b) is performed in the presence of a solvent. The solvent may typically comprise one or more solvents which are unreactive with the selection of compounds used in step (b). In some aspects, the solvent comprises an ethereal solvent, for example diethyl ether, tetrahydrofuran, 1,4-dioxane, cyclopentyl methyl ether, or combinations thereof. Other suitable solvents may be used as would be apparent to a person of skill in the art. In some aspects, step (b) is performed at a temperature ranging from about −10 degrees Celsius to about 30 degrees Celsius.

In another aspect, a process is provided for synthesis of a compound of Formula VII-a

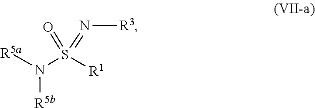

the process comprising:
(a) preparing a compound of Formula V according to the processes described herein;
(b) reacting the compound of Formula V with a compound of Formula VI-a in the presence of an amine base

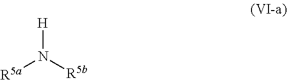

to form the compound of Formula VII;
wherein:
R^{5a} is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5a} is optionally substituted with one or more Z groups as allowed by valency;
R^{5b} is selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5b} is optionally substituted with one or more Z groups as allowed by valency;
or R^{5a} and R^{5b} are brought together with the nitrogen to which they are attached to form a 3- to 8-membered monocyclic or bicyclic heterocycle which is optionally substituted with one or more Z groups as allowed by valency; and
all other variables are as defined herein.

In some aspects, the amine base is selected from pyridine, 2,6-lutidine, collidine, triethylamine, tributylamine, and quinuclidine. In some aspects, step (b) is performed in the presence of a lithium salt, such as lithium chloride or lithium bromide.

In some aspects, step (b) is performed in the presence of a solvent. The solvent may typically comprise one or more solvents which are unreactive with the selection of compounds used in step (b). In some aspects, the solvent comprises acetonitrile. Other suitable solvents may be used as would be apparent to a person of skill in the art. In some aspects, step (b) is performed at a temperature ranging from about 25 degrees Celsius to about 100 degrees Celsius.

In another aspect, a compound is provided of Formula VII prepared by the processes described herein. In yet another aspect, a compound is provided of Formula VII-a prepared by the processes described herein.

### Processes for Synthesis of Compounds of Formula VIII and Formula VIII-a

In another aspect, a process is provided of synthesis of a compound of Formula VIII

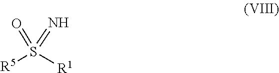

the process comprising:
(a) preparing a compound of Formula VII according to the processes described herein; and
(b) reacting the compound of Formula VIII in the presence of an acid or by heating to form the compound of Formula VIII;
wherein all variables are as defined herein.

In another aspect, a process is provided of synthesis of a compound of Formula VIII-a

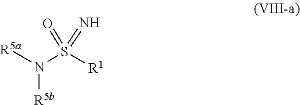

(a) preparing a compound of Formula VII according to the process of any one of claims 53-58; and
(b) reacting the compound of Formula VIII in the presence of an acid or by heating to form the compound of Formula VIII;
wherein all variables are as defined herein.

In some aspects, the acid comprises camphor sulfonic acid. In some aspects, step (b) is performed in the presence of a solvent. The solvent may typically comprise one or more solvents which are unreactive with the selection of compounds used in step (b). In some aspects, the solvent comprises hexafluoroisopropanol. In some aspects, the solvent comprises a mixture of dimethyl sulfoxide and water. Other suitable solvents may be used as would be apparent to a person of skill in the art.

In another aspect, a compound is provided of Formula VIII or Formula VIII-a prepared by the processes described herein.

Variations on compounds of Formula I, Formula II, Formula III, Formula IV, Formula V, Formula VI, Formula VI-A, Formula VII, Formula VII-a, Formula VIII, Formula VIII-a as made by or used in the processes described herein can include the addition, subtraction, or movement of the various constituents as described for each compound. Similarly, when one or more chiral centers is present in a molecule the chirality of the molecule can be changed. Additionally, the synthesis of the compounds for use in the process can involve the protection of various chemical groups, and further the compounds prepared by the disclosed processes may be subsequently deprotected as appropriate. The use of protection and deprotection, and the selection of appropriate protecting groups would be readily known to one skilled in the art. The chemistry of protecting groups can be found, for example, in Peter G. M. Wuts, Greene's Protective Groups in Organic Synthesis, 5th Ed., Wiley & Sons, 2014.

In some embodiments of the disclosed processes, the sulfur atom found in the compounds described herein is chiral. In some embodiments, the chirality of the sulfur atom may be inverted during any of the processes described herein. In some embodiments, the processes describe herein allow the asymmetric synthesis of the produced compounds.

The described processes, or reactions to produce the compounds used in the described processes, can be carried out in solvents indicated herein, or in solvents which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high-performance liquid chromatography (HPLC) or thin layer chromatography.

In another aspect, the following particular embodiments of the present disclosure are also provided:
Embodiment 1. A compound of Formula II:

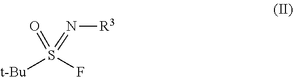

wherein R³ is —C(═O)—N(R⁴) 2, and wherein R⁴ is independently selected at each occurrence from C₃-C₅ alkyl.
Embodiment 2. The compound of embodiment 1, wherein each R⁴ is isopropyl.
Embodiment 3. The compound of embodiment 1 or embodiment 2, wherein the compound is selected from

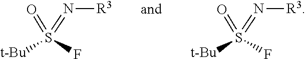

Embodiment 4. A compound of Formula II-a

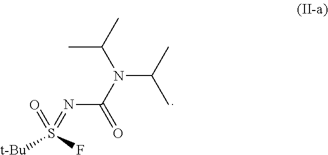 [SMILES: CC(C)N(C(=O)N=[S@](=O)(F)C(C)(C)C)C(C)C]

Embodiment 5. A compound of Formula II-b

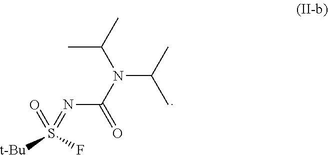 [SMILES: CC(C)N(C(=O)N=[S@@](=O)(F)C(C)(C)C)C(C)C]

Embodiment 6. A process for synthesis of a compound of Formula I

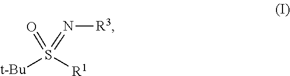

the process comprising:
reacting a compound of Formula II

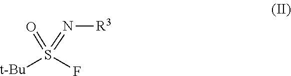

with a compound of Formula III

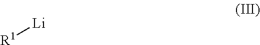

to form the compound of Formula I;
wherein:
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.
Embodiment 7. The process of embodiment 6, wherein each R⁴ is independently selected from isopropyl, sec-butyl, tert-butyl, and isopentyl.
Embodiment 8. The process of embodiment 6 or embodiment 7, wherein each R⁴ is isopropyl.
Embodiment 9. The process of any one of embodiments 6-8, wherein the compound of Formula II is selected from:

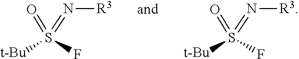

Embodiment 10. The process of any one of embodiments 6-9, wherein the process is performed in the presence of a solvent.
Embodiment 11. The process of embodiment 10, wherein the solvent comprises an ethereal solvent.
Embodiment 12. The process of embodiment 10 or embodiment 11, wherein the solvent comprises diethyl ether, tetrahydrofuran, 1,4-dioxane, cyclopentyl methyl ether, or combinations thereof.
Embodiment 13. The process of any one of embodiments 6-12, wherein the process is performed at a temperature from about-80 degrees Celsius to about-40 degrees Celsius.
Embodiment 14. A compound of Formula I prepared according to the process of any one of embodiments 6-13.
Embodiment 15. A process for synthesis of a compound of Formula V

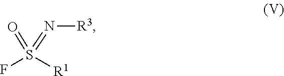

the process comprising:
(a) preparing a compound of Formula according to the process of any one of embodiments 6-13;
(b) reacting the compound of Formula I with a first base to form a compound of Formula IV

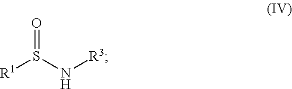

and
(c) reacting the compound of Formula IV with a fluorinating reagent and a second base to form the compound of Formula V

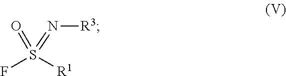

wherein all variables are as defined in embodiment 6.
Embodiment 16. The process of embodiment 15, wherein the first base comprises a metal alkoxide.
Embodiment 17. The process of embodiment 15 or 16, wherein the first base comprises a metal tert-butoxide, such as sodium or potassium tert-butoxide.
Embodiment 18. The process of any one of embodiments 15-17, wherein step (b) is performed in the presence of a solvent.
Embodiment 19. The process of embodiment 18, wherein the solvent comprises an ethereal solvent.
Embodiment 20. The process of embodiment 18 or 19, wherein the solvent comprises diethyl ether, tetrahydrofuran, 1,4-dioxane, cyclopentyl methyl ether, or combinations thereof.
Embodiment 21. The process of any one of embodiments 15-20, wherein step (b) is performed at a temperature from about 25 degrees Celsius to about 100 degrees Celsius.
Embodiment 22. The process of any one of embodiments 15-21, wherein the fluorinating agent comprises an electrophilic fluorinating agent.
Embodiment 23. The process of any one of embodiments 15-22, wherein the fluorinating agent comprises N-fluorobenzenesulfonimide.
Embodiment 24. The process of any one of embodiments 15-23, wherein the second base comprises a metal hydride.
Embodiment 25. The process of any one of embodiments 15-24, wherein the second base comprises sodium hydride, potassium hydride, or cesium hydride.
Embodiment 26. The process of any one of embodiments 15-25, wherein step (c) is performed in the presence of a solvent.
Embodiment 27. The process of embodiment 26, wherein the solvent comprises an ethereal solvent.
Embodiment 28. The process of embodiment 26 or 27, wherein the solvent comprises diethyl ether, tetrahydrofuran, 1,4-dioxane, cyclopentyl methyl ether, or combinations thereof.
Embodiment 29. The process of any one of embodiments 15-28, wherein step (c) is performed at a temperature ranging from about-40 degrees Celsius to about 30 degrees Celsius.
Embodiment 30. A process for synthesis of a compound of Formula V

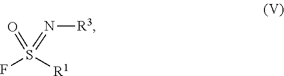

the process comprising:
(a) preparing a compound of Formula I according to the process of any one of embodiments 6-13;
(b) reacting the compound of Formula I with a first base to form a reaction mixture;
(c) reacting the first reaction mixture with a fluorinating reagent in the presence of an acid to form the compound of Formula V

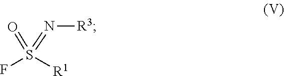

wherein all variables are as defined in embodiment 6.
Embodiment 31. The process of embodiment 30, wherein the first base comprises a metal alkoxide.
Embodiment 32. The process of embodiment 30 or 31, wherein the first base comprises a metal tert-butoxide, such as sodium or potassium tert-butoxide.
Embodiment 33. The process of any one of embodiments 30-32, wherein step (b) is performed at a temperature from about 25 degrees Celsius to about 100 degrees Celsius.
Embodiment 34. The process of any one of embodiments 30-33, wherein step (b) is performed in the presence of a solvent.
Embodiment 35. The process of embodiment 34, wherein the solvent comprises an ethereal solvent.
Embodiment 36. The process of embodiment 34 or 35, wherein the solvent comprises diethyl ether, tetrahydrofuran, 1,4-dioxane, cyclopentyl methyl ether, or combinations thereof.
Embodiment 37. The process of any one of embodiments 30-36, wherein the reaction mixture formed in step (b) is not isolated or purified before performing step (c).
Embodiment 38. The process of any one of embodiments 30-37, wherein the fluorinating agent comprises an electrophilic fluorinating agent.
Embodiment 39. The process of any one of embodiments 30-38, wherein the fluorinating agent comprises N-fluorobenzenesulfonimide.
Embodiment 40. The process of any one of embodiments 30-39, wherein the acid comprises an organic acid.
Embodiment 41. The process of any one of embodiments 30-40, wherein the acid comprises acetic acid.
Embodiment 42. The process of any one of embodiments 30-41, wherein step (c) is performed at a temperature ranging from about-40 degrees Celsius to about 30 degrees Celsius.
Embodiment 43. A compound of Formula V prepared according to the process of any one of embodiments 15-42.
Embodiment 44. A process for synthesis of a compound of Formula VII

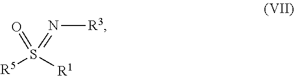

the process comprising:
(a) preparing a compound of Formula V according to the process of any one of embodiments 15-42;
(b) reacting the compound of Formula V with a compound of Formula VI

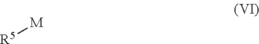

to form the compound of Formula VII;
wherein:
R⁵ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, and (R^{x}R^{y}N)—(C₀-C₃ alkyl)-, wherein each R⁵ is optionally substituted with one or more Z groups as allowed by valency;
M is selected from Li, Na, MgX¹, and MgX¹LiX², wherein X¹ and X² are each independently halo; and
all other variables are as defined in embodiment 6.
Embodiment 45. The process of embodiment 44, wherein M is Li.
Embodiment 46. The process of embodiment 44, wherein M is Na.
Embodiment 47. The process of embodiment 44, wherein M is MgCl or MgBr.
Embodiment 48. The process of embodiment 44, wherein M is MgBrLiCl or MgClLiCl.
Embodiment 49. The process of any one of embodiments 44-48, wherein step (b) is performed in the presence of a solvent.
Embodiment 50. The process of embodiment 49, wherein the solvent comprises an ethereal solvent.
Embodiment 51. The process of embodiment 49 or 50, wherein the solvent comprises diethyl ether, tetrahydrofuran, 1,4-dioxane, cyclopentyl methyl ether, or combinations thereof.
Embodiment 52. The process of any one of embodiments 35-62, wherein step (b) is performed at a temperature ranging from about-10 degrees Celsius to about 30 degrees Celsius.
Embodiment 53. A process for synthesis of a compound of Formula VII-a

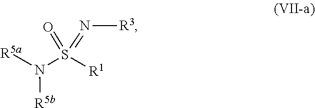

the process comprising:
(a) preparing a compound of Formula V according to the process of any one of embodiments 15-42;
(b) reacting the compound of Formula V with a compound of Formula VI-a in the presence of an amine base

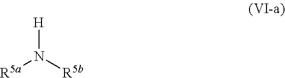

to form the compound of Formula VII;
wherein:
R^{5a} is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5a} is optionally substituted with one or more Z groups as allowed by valency;
R^{5b} is selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5b} is optionally substituted with one or more Z groups as allowed by valency;
or R^{5a} and R^{5b} are brought together with the nitrogen to which they are attached to form a 3- to 8-membered monocyclic or bicyclic heterocycle which is optionally substituted with one or more Z groups as allowed by valency; and all other variables are as defined in embodiment 6.
Embodiment 54. The process of embodiment 53, wherein the amine base is selected from pyridine, 2,6-lutidine, collidine, triethylamine, tributylamine, and quinuclidine.
Embodiment 55. The process of embodiment 53 or embodiment 54, wherein step (b) is performed in the presence of a lithium salt, such as lithium chloride or lithium bromide.
Embodiment 56. The process of any one of embodiments 53-55, wherein step (b) is performed in the presence of a solvent.
Embodiment 57. The process of embodiment 56, wherein the solvent comprises acetonitrile.
Embodiment 58. The process of any one of embodiments 53-57, wherein step (b) is performed at a temperature ranging from about 25 degrees Celsius to about 100 degrees Celsius.
Embodiment 59. A compound of Formula VII prepared by the process of any one of embodiments 44-52.
Embodiment 60. A compound of Formula VII-a prepared by the process of any one of embodiments 53-58.
Embodiment 61. A process of synthesis of a compound of Formula VIII

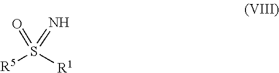

the process comprising:
(a) preparing a compound of Formula VII according to the process of any one of embodiments 44-52; and
(b) reacting the compound of Formula VIII in the presence of an acid or by heating to form the compound of Formula VIII;
wherein all variables are as defined in embodiment 44.
Embodiment 62. A process of synthesis of a compound of Formula VIII-a

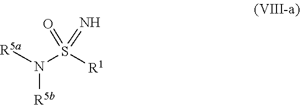

(a) preparing a compound of Formula VII according to the process of any one of embodiments 53-58; and
(b) reacting the compound of Formula VIII in the presence of an acid or by heating to form the compound of Formula VIII;
wherein all variables are as defined in embodiment 53.
Embodiment 63. The process of embodiment 61 or embodiment 62, wherein the acid comprises camphor sulfonic acid.
Embodiment 64. The process of any one of embodiments 61-63, wherein step (b) is performed in the presence of a solvent.
Embodiment 65. The process of embodiment 64, wherein the solvent comprises hexafluoroisopropanol.
Embodiment 66. The process of embodiment 64, wherein the solvent comprises a mixture of dimethyl sulfoxide and water.
Embodiment 67. A compound of Formula VIII or Formula VIII-a prepared by the process of any one of embodiments 61-66.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compounds, compositions, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### General Experimental.

Reagents were purchased at the highest commercial quality and used without further purification, unless otherwise stated. Anhydrous diethyl ether (Et₂O), tetrahydrofuran (THF), 2-methyl tetrahydrofuran (2-Me-THF), toluene (PhMe) were obtained by passing the previously degassed solvent through an activated alumina column (PPT Glass Contour Solvent Purification System). Anhydrous cyclopentyl methyl ether (CPME), dimethoxyethane (DME) and methyl tert-butyl ether (MTBE) were purchased from Acros Organics. All glassware was flame-dried under vacuum before use. Yields refer to chromatographically and spectroscopically (¹H NMR) homogeneous material, unless otherwise stated. Reactions were monitored by LC-MS or thin layer chromatography (TLC) carried out on 250 μm SiliCycle SiliaPlates (TLC Glass-Backed TLC Extra Hard Layer, 60 Å), using shortwave UV light as the visualizing agent and p-anisaldehyde, phosphomolybdic acid (PMA) or KMnO₄ with heat as developing agents. Flash column chromatography was performed with a Biotage Isolera One (ZIP or SNAP Ultra cartridges) or with traditional glass flash columns using SiliCycle SiliaFlash® P60 (particle size 40-63 μm). NMR spectra were recorded on a Bruker Ascend™ 500 MHz instrument or Bruker Neo600 600 MHz spectrometer and were calibrated using residual undeuterated solvent as an internal reference (CDCl₃: 7.26 ppm ¹H NMR, 77.16 ppm ¹³C NMR; DMSO-d6:2.50 ppm ¹H NMR, 39.5 ppm ¹³C NMR). The following abbreviations were used to explain NMR peak multiplicities: s=singlet, d=doublet, t=triplet, q=quartet, dd=doublet of doublet, ddd=doublet of doublet of doublet, dddd=doublet of doublet of doublet of doublet, ddddd=doublet of doublet of doublet of doublet of doublet, tt=triplet of triplet, ddt=doublet of doublet of triplet, m=multiplet, br=broad, hept=heptet. High resolution mass spectra (HRMS) were recorded on an Agilent 6230 LC-MS TOF mass spectrometer. Enantiomeric excess (ee) was determined using a Varian Prostar HPLC with a 210 binary pump and a 335 diode array detector. Optical rotations were measured using a JASCO P-2000 polarimeter with a cell length of 1 dm. Melting points were recorded on a Chemglass DMP 100 melting point apparatus and were uncorrected.

### Handling of Reagents.

All synthesized sulfonimidoyl fluorides were stored under ambient conditions, either room temperature or at −20° C. Notably, there is no significant difference observed regarding to reactivity and stereospecificity. Specifically for the t-BuSF, NMR tests demonstrated it is stable for at least 7-month under ambient conditions—in other words, it is bench stable. Sulfoximines and sulfonimidamides were stored under ambient conditions and appeared to be unchanged over the course of this work.

Synthesis of Enantiopure Bifunctional Sulfonimidoyl Transfer Reagent t-BuSF.

### Scalable Synthesis of Diisopropyl Carbamoyl Chloride (DIP-CCI).

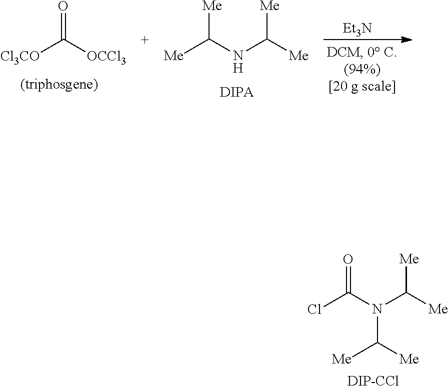

In a 1 L round-bottomed flask equipped with a stir bar, septum capped addition funnel (500 mL) and argon balloon was added triphosgene (20 g, 16.9 mmol, 1 eq.) and DCM (100 mL) then cooled to 0° C. A solution of DIPA (20.5 g, 28.6 mL, 202 mmol, 3 eq.) and Et₃N (20.5 g, 28.2 mL, 202 mmol, 3 eq.) in DCM (240 mL) was added to the addition funnel (directly poured using a funnel and recapped) then added over 10 minutes. The reaction mixture stirred at 0° C. for 1.5 hours, removed from the ice bath, filtered through a sintered glass funnel (removal of Et₃N—HCl) and rinsed with DCM (50 mL×3). The filtrate was concentrated under reduced pressure to remove DCM (rotary evaporator bath set to 25° C.) then taken up in hexanes (450 mL), washed with water (3×200 mL), dried over Na₂SO₄, filtered and concentrated (rotary evaporator bath set to 25° C.) to give the desired carbamoyl chloride (31.1 g, 190 mmol, 94% yield) as an off-white solid that was sufficiently pure by NMR and used in the next step without further purification.

Physical characteristics: Off-white solid ¹H NMR: (500 MHz, CDCl₃) δ 4.53 (q, J=6.9 Hz, 1H), 3.66-3.50 (m, 1H), 1.36 (d, J=6.9 Hz, 6H), 1.21 (d, J=6.9 Hz, 6H) ppm. 13C NMR: (126 MHZ, CDCl₃) δ 145.98, 52.89, 48.55, 20.37, 19.94 ppm

General Procedure for a Decagram Synthesis of Enantiopure t-BuSF.

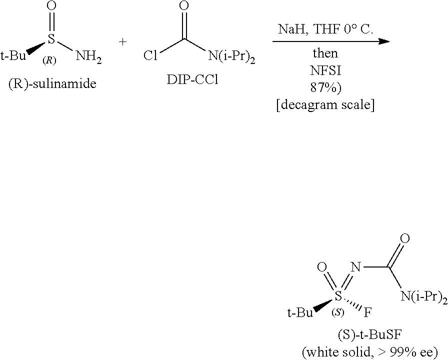

In a septum capped 1 L round-bottomed flask equipped with a stir bar and argon balloon was added (R)-sulfinamide (10.3 g, 41.4 mmol, 1 eq.) and THF (400 mL, 0.21 M) then cooled to 0° C. NaH (8.50 g, 212 mmol, 2.5 eq., 60% wt) was added portion-wise (3 portions) then stirred for 20 minutes until H₂ gas evolution ceased. DIPC-CCI (13.9 g, 84.9 mmol, 1 eq.) was added portion-wise (3 portions) then stirred at 0° C. for 1.5 hours until H₂ gas evolution ceased (reaction monitored by TLC and LC-MS for the disappearance of sulfinamide). NFSI (28.1 g, 89.2 mmol, 1.05 eq.) was added in one portion then stirred at 0° C. for an additional 1 hour (reaction monitored by TLC and LC-MS for the disappearance of sulfinyl urea intermediate). The reaction mixture was removed from the ice bath and diluted with 10% EtOAc in hexanes (300 mL) then filtered through a medium porous sintered glass funnel while rinsing with 10% EtOAc in hexanes. The organic solution was washed with 10% KI aqueous solution (150 mL×3: for removal of unreacted NFSI) and brine (150 mL×3). The solvent was dried over anhydrous Na₂SO₄, filtered and solvents removed under reduced pressure to give a yellow oil. Further purification by silica gel column chromatography using hexanes/EtOAc (0% to 20% EtOAc) provided the desired sulfonimidoyl fluoride (19.8 g, 74.3 mmol, 87% yield) as a clear colorless oil that solidified to a white crystalline solid under reduced pressure.

Physical characteristics: White crystalline solid TLC: R_{f}=0.33 (hexane/EtOAc, 20% EtOAc, PMA). ¹H NMR: (500 MHZ, CDCl₃) δ 4.03 (s, 1H), 3.88 (s, 1H), 1.59 (d, J=0.7 Hz, 9H), 1.38-1.10 (m, 12H) ppm. ¹³C NMR: (126 MHz, CDCl₃) δ 153.88, 62.76 (d, J=11.7 Hz), 47.83, 45.86, 24.72, 21.36, 20.67, 20.59 ppm. 19F NMR: (471 MHZ, CDCl₃) δ 33.20 ppm. Specific rotation: [α]23/D=+78.17 (c 1.00, CHCl₃) HRMS: Calc'd for C₁₁H₂₃FN₂NaO₂S [M+Na⁺] 289.1356; found 289.1363. Enantiomeric excess: >99% HPLC Conditions: Daicel Chiralpak IC column, 70:30 n-hexane:i-PrOH, flow rate: 1 mL min-1, 25° C., UV detection wavelength: 220 nm, retention time: minor: 13.02 min, major: 16.67 min.

Stability Experiments for t-BuSF Reagent
Bench Stability Analysis of t-BuSF.

The t-BuSF used for the stability study was prepared following the general procedure and stored on the lab bench in a capped 20 mL colorless vial at ambient temperatures. Room temperature was variable (23-26° C. and humidity was roughly 55%). t-BuSF samples were prepared from the same batch, separated into 3 different vials and compared with freshly prepared material. Stability analysis using ¹H-NMR, ¹⁹F-NMR and ¹³C-NMR were used and accompanied by a reaction performance analysis (% yield and % ee) using phenyl lithium as the nucleophile.

Based on the NMR analysis, no significant decomposition was observed. The only identifiable peaks were that of diisopropyl amine (presumably forms via decomposition pathway). Reaction performance analysis provided identical enantiopurity of products with slightly diminished yields (up to 10%).

### Thermal Stability of Tert-Butyl Sulfonimidoyl Fluoride Reagents.

An analysis of thermal stability of t-BuSF was performed and compared to other protected t-Bu sulfonimidoyl fluorides. Three different solvents were used for a thermal distribution of 35-110° C. Each sample (0.2 mmol, 0.1 M) was refluxed in the respective solvent: Et₂₀ (35° C.), THF (66° C.) and toluene (110° C.) for 24 hours. Note: anhydrous solvents and conditions were not employed. After 24 hours of heating, the reactions were cooled to room temperature, the solvent was removed under reduced pressure, and the remaining residue dissolved in CDCl₃ then analyzed by NMR (1H and 1° F.).

Thermal Stability of t-BuSF.

Based on the NMR analysis of t-BuSF, significant decomposition was only observed when heating at 110° C. in toluene for 24 hours. We were unable to identify the by-products besides diisopropyl amine. Decomposition was not observed at room temperature in either of the solvents used.

### Thermal Stability Comparison of Different Protecting Groups.

Four different protecting groups were evaluated for thermal stability: two urea-based (—CON(i-Pr)₂, —CONEt₂) one carbamate (-Boc) and one acyl (-Piv). Other protecting groups including silyl (-TBS, -TBDPS), -tosyl, and -benzyl were unable to be prepared and evaluated due to reactivity or stability issues of the sulfinamide precursors and/or sulfonimidoyl fluoride products.

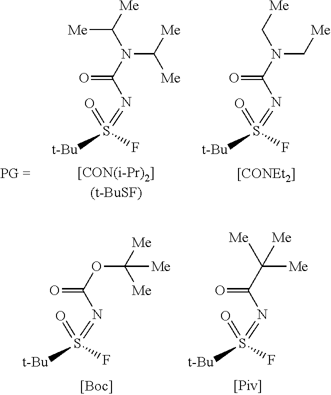 [SMILES: CC(C)N(C(=O)N=[S@@](=O)(F)C(C)(C)C)C(C)C ; CCN(CC)C(=O)N=[S@@](=O)(F)C(C)(C)C ; CC(C)(C)OC(=O)N=[S@@](=O)(F)C(C)(C)C ; CC(C)(C)C(=O)N=[S@@](=O)(F)C(C)(C)C]

Experimental procedures and analyses were identical to those described above for the t-BuSF reagent. ¹H NMR was found to provide greater diagnostic evidence of decomposition compared to 19F NMR. The urea protecting groups were both found to have increased stability under the thermal conditions relative to carbamate and acyl protecting groups. Both -Boc and -Piv protecting groups exhibited significant decomposition after refluxing (66° C.) in THF for 24 hours. No obvious decomposition was observed when refluxing (35° C.) in Et₂O for 24 hours across all four protected sulfonimidoyl fluorides.

Synthesis of N,N-diisopropyl urea protected chiral tert-butyl sulfoximines: first functionalization of t-BuSF.

### Optimization for the Synthesis of N,N-Diisopropyl Urea Protected Chiral Tert-Butyl Aryl Sulfoximines Using Phenyl Lithium as a Model Nucleophile.

TABLE 1
All reactions were performed on a 0.3 mmol scale and
t-BuSF added to PhLi (1.9M in dibutyl ether) at −78 ° C.
Reactions were quenched within 1 hour after addition unless
otherwise stated. Enantiopurity was determined by chiral HPLC.

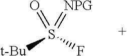

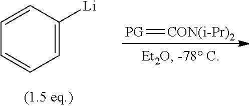

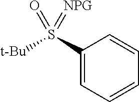

| | | Enantiopurity | Yield |
|---|---|---|---|
| Entry | Variations | (% ee) | (%)^{a} |
| 1 | None^{c} | 98 | 87 |
| 2 | THF | NAb | <5 |
| 3 | 2-Me-THF | 96 | 15 |
| 4 | CPME | 98 | 80 |
| 5 | DME^{d} | 96 | 75 |
| 6 | MTBE | 95 | 42 |
| 7 | PhMe | 96 | 50 |
| 8 | dibutyl ether | 95 | 45 |
| 9 | Et₂O/THF = 8:1 | 97 | 67 |
| 10 | Et₂O/hexane = 8:1 | 96 | 60 |
| 11 | PhLi (1.0 eq.) | 98 | 55 |
| 12 | 0° C. | 91 | 83 |
| 13 | −50° C. | 96 | 85 |
| 14 | TMEDA (1.5 eq.) as additive | 93 | 75 |
| 15 | HMPA (1.0 eq.) as additive | 96 | 74 |
| 16 | HMPA (10 eq.) as additive | NA^{b} | 0 |
| 17 | DMPU (1 eq.) as additive | 97.4 | 74 |
| 18 | TBAB (1.0 eq.) as additive | 95 | 73 |
| 19 | TBAI (1.0 eq.) as additive | 96 | 73 |
| 20 | LiCIO4 (1.0 eq.) as additive | 98.2 | 48 |
| 21 | LiCIO4 (2.0 eq.) as additive | 96.2 | 32 |
| 22 | NFSI (0.15 eq.) as additive | 91.3ª | 79 |
| 23 | t-BuSF added (0.1 mL/min) | 98.4 | 81 |
| 24 | t-BuSF added (0.05 mL/min) | 98.3 | 83 |
| 25 | t-BuSF added (0.025 mL/min) | 98.3 | 81 |
| 26 | t-BuSF added (0.01 mL/min), 0.05M | 98.2 | 75 |
| 27 | t-BuSF added (0.01 mL/min), 0.2M | 98.3 | 74 |
| 28 | PhLi added (0.1 mL/min) | 97.6 | 80 |
| 29 | PhLi added (0.05 mL/min) | 98.2 | 92 |
| 30 | PhLi added (0.025 mL/min) | 98.4 | 83 |
| 31 | PhLi added (0.01 mL/min) | 98.5 | 91 |
| 32 | PhLi added (0.01 mL/min), 0.05M | 98.2 | 81 |
| 33 | PhLi added (0.01 mL/min), 0.2M | 97.5 | 73 |
| 34 | PG = CON(Me)₂ | NA^{b} | 0 |
| 35 | PG = CON(Et)₂ | NA^{b} | <5 |
| 36 | PG = Piv | NA^{b} | 0 |
| 37 | PG = Boc | NA^{b} | <5 |
| 38 | PG = Bz | NA^{b} | 0 |

ªIsolated yield.
^{b}Not available.
^{c}No observable difference in yield or % ee when forming PhLi in situ using GP-1 or GP-2.
^{d}1,2-dimethoxyethane (DME) was warmed to −50° C. due to its melting point.
^{e}t-BuSF (93.5% ee) prepared using Selectfluor instead of NFSI. Addition rates (entries 23-33) were controlled via syringe pump.

### General Procedures for the Synthesis of Chiral N,N-Diisopropyl Urea Protected Tert-Butyl Sulfoximines.

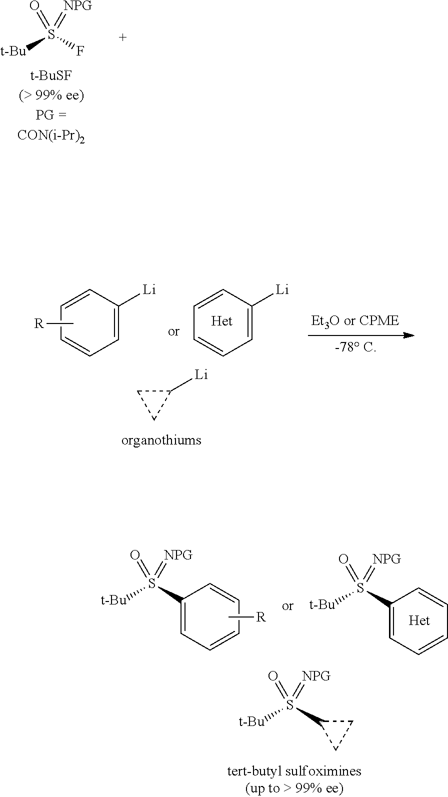

### General Procedure 1 (GP-1): Stepwise Addition of Reagents.

To a 10 mL flame dried round-bottomed flask equipped with magnetic stir bar and argon balloon was added aryl bromide (0.375 mmol, 1.5 eq.) and anhydrous Et₂O (2.0 mL). Cooled to −78° C. and n-BuLi (0.150 mL, 2.5 M in hexane, 1.5 eq.) was added dropwise. Stirred for 1 hour (lithium-halogen exchange) then tert-butyl sulfonimidoyl fluoride (66.5 mg, 0.25 mmol, 1 eq.) in Et₂O (0.5 mL) was added dropwise. The reaction mixture stirred at −78° C. for 1 hour (unless otherwise stated-vide infra). Upon completion (checked by TLC and LC-MS) MeOH (0.2 mL) and saturated aqueous NH₄Cl (5 mL) were added to quench the reaction. The mixture was transferred to separatory funnel and extracted with EtOAc (5 ml×3), then washed with water (10 mL×3) and brine (10 mL×3). Dried over anhydrous Na₂SO₄, filtered and concentrated under reduced pressure. Further purification was performed by silica gel column chromatography to give the desired tert-butyl sulfoximines.

General Procedure 2 (GP-2): Lithiation of Aryl Halides in the Presence of t-BuSF.

To a 10 mL flame dried round-bottomed flask equipped with magnetic stir bar and argon balloon was added aryl bromide (0.375 mmol, 1.5 eq.), t-Bu sulfonimidoyl fluoride (66.5 mg, 0.25 mmol, 1 eq.) and anhydrous Et₂₀ (2.5 mL). Cooled to −78° C. and 1-BuLi (0.22 mL, 1.7 M in pentane, 1.5 eq.) was added dropwise. The reaction mixture was stirred at −78° C. for 1 hour (some substrates required longer Li—X exchange times; vide infra). Upon completion (checked by TLC and LC-MS) MeOH (0.2 mL) and saturated aqueous NH₄Cl (5 mL) were added to quench the reaction. The mixture was transferred to separatory funnel and extracted with EtOAc (5 ml×3), then washed with water (10 mL×3) and brine (10 mL×3). Dried over anhydrous Na₂SO₄, filtered and concentrated under reduced pressure. Further purification was performed by silica gel column chromatography to give the desired tert-butyl sulfoximines.

### General Procedure 3 (GP-3): Lithium-Halogen Exchange of (Hetero) Aryl Halides and Substrates Less Prone to Lithiation.

To a flame dried round-bottomed flask equipped with magnetic stir bar under argon was added (hetero) aryl halide (0.375 mmol, 1.5 eq.) and Et₂O (2.0 mL) then cooled to −78° C. n-BuLi (0.150 mL, 0.375 mmol, 2.5 M in hexane, 1.5 eq.) was added dropwise and stirred at −78° C. for 30 minutes then warmed to −20-0° C. gradually and stirred for another 30 minutes then cooled to −78° C. A solution of t-BuSF (0.25 mmol, 1 eq.) in Et₂₀ (0.5 mL) was added dropwise. The reaction mixture was stirred at −78° C. for 1 hour (or warmed to the temperature noted below). Upon completion (checked by TLC and LC-MS) MeOH (0.2 mL) and saturated aqueous NH₄Cl (5 mL) were added to quench the reaction. The mixture was transferred to separatory funnel and extracted with EtOAc (5 ml×3), then washed with water (10 mL×3) and brine (10 mL×3). Dried over anhydrous Na₂SO₄, filtered and concentrated under reduced pressure. Further purification was performed by silica gel column chromatography to give the desired tert-butyl sulfoximines.

### Activating Urea Protected Tert-Butyl Sulfoximines for Further Functionalization.

### Optimization for the Reductive De-Tert-Butylation of N,N-Diisopropyl Urea Protected Chiral Tert-Butyl Sulfoximines to Sulfinyl Ureas.

TABLE 2
All reactions were performed on a 0.3 mmol scale in
a flame dried flask under argon with anhydrous solvents.
Enantiopurity was determined by using chiral HPLC.
Reactions were quenched with wet (THF/DCM) silica gel.

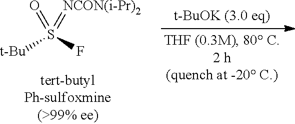

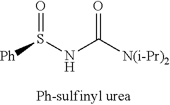

| | | Enantiopurity | Yield |
|---|---|---|---|
| Entry | Variations | (% ee) | (%)^{a} |
| 1 | None | >99 | 88 |
| 2 | 4 h | >99 | 87 |
| 3 | 8 h | >99 | 88 |
| 4 | dioxane | >99 | 60 |
| 5 | DMF | 91 | 75 |
| 6 | quench at 0° C. | 97 | 84 |
| 7 | quench at rt | 93 | 83 |
| 8 | TFA (0.2-1.5 eq.), | NA | decomposed |
| | DCM (0.25M), rt | | |
| 9 | 4.0M HCl in Dioxane (5-20 eq.) | NA | decomposed |
| | DCM (0.2M), −78 to 0° C. | | |
| 10 | BF₃•OEt₂ (1.0 eq.), | NA | decomposed |
| | THF (0.1M), 0 to 50° C.^{b} | | |
| 11 | Mg(ClO₄)₂ (1.0 eq.), | NA | NR |
| | THF (0.2M), rt to 80° C.^{b} | | |
| 12 | BH₃ (2.0 eq.), THF (0.2M), rt | NA | NR |
| 13 | Selectfluor (2.0 eq.), MeCN (0.1M) | NA | (<20%)^{c} |
| | rt to 50° C., 48 h | | |

ªIsolated yield. ^{b}Decomposed upon heating. ^{c}Relative yield based on LC-MS.

General Procedure 4 (GP-4): Reductive De-Tert-Butylation of N,N-Diisopropyl Urea Protected Chiral Tert-Butyl Sulfoximines to Sulfinyl Ureas Using t-BuOK.

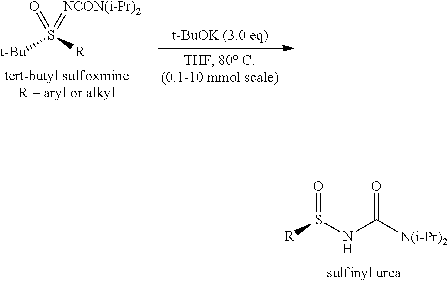

In a septum capped flame dried round-bottomed flask equipped with a stir bar and argon balloon was added tert-butyl sulfoximine (1 eq.) followed by THF (0.3 M). Solid t-BuOK (3 eq.) was added, and the reaction stirred at room temperature for 2-5 minutes. The argon balloon was removed, and the reaction placed in a pre-heated oil bath set to 80° C. for 2 hours (behind a blast shield). Upon competition (checked by TLC) the reaction was cooled to −20° C. then quenched by adding a solution of AcOH (2 eq.) in THF (1-2 M) followed by silica gel (10:1, silica gel to starting material by mass) or by adding wet (THF and DCM) silica gel (20:1, silica gel to starting material by mass) with continual stirring at −20° C. for 2-5 minutes. The reaction mixture containing silica was filtered a plug of silica gel (wet with DCM) and rinsed with DCM. The filtrate was concentrated under reduced pressure via rotary evaporated with a water bath set to 25° C. to give the desired sulfinyl urea in 80-90% yields with high purity.

Optimization for the S-fluorination of chiral sulfinyl ureas to N,N-diisopropyl urea protected chiral sulfonimidoyl fluorides.

TABLE 3
All reactions were performed on 0.3 mmol scale in a flame
dried flask under argon. Enantiopurity was determined by
using chiral HPLC.

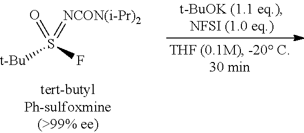

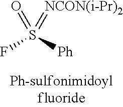

| | | Enantiopurity | Yield |
|---|---|---|---|
| Entry | Variations | (% ee) | (%)^{a} |
| 1 | None | >99 | 91 |
| 2 | 0° C., 5 min | 98 | 60 |
| 3 | 0° C., 30 min | 98 | 93 |
| 4 | −78° C., 2 h | >99 | 65 |
| 5 | DMF | 96 | 81 |
| 6 | NaH (1.5 eq.), 0° C. | 97 | 90 |
| 7 | NaH (1.5 eq.), −20° C. | >99 | 89 |
| 8 | EtOH (0.2M), AcOK (2.0 eq.) | | 87 |
| | selectfluor(2.0 eq.), 0° C. | | |
| | to rt, 24 h | 97 | |
| 9 | DME, 0° C., NaOH (1.5 eq.) | 98 | 83 |

ªIsolated yield.

General Procedure 5 (GP-5): Synthesis of N,N-Diisopropyl Urea Protected Chiral Sulfonimidoyl Fluorides from Sulfinyl Ureas.

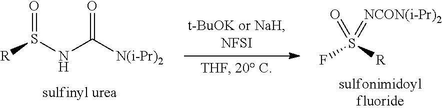

In a septum capped flask or reaction vial equipped with a stir bar and argon balloon was added sulfinyl urea (1 eq.) and THF (0.1 M) then cooled to −20° C. Either solid t-BuOK (1.1 eq.) or NaH (1.5 eq., 60% wt) was added (in one portion for small scale reactions, portion wise for >5 mmol scales) and the reaction stirred for 15 minutes at −20° C. NFSI (1 eq.) was added in one portion and the reaction continued to stir for 30 minutes at −20° C. Upon completion (checked by TLC) the reactions were quenched with saturated aqueous NH₄Cl and extracted with EtOAc (×3). Combined organic layers were washed with water and brine, dried over Na₂SO₄, filtered and concentrated. Further purification by silica gel column chromatography provided the desired sulfonimidoyl fluorides.

### Optimization of the One-Pot Transformation of N,N-Diisopropyl Urea Protected Chiral Tert-Butyl Sulfoximines to Sulfonimidoyl Fluorides.

TABLE 4
All reactions were performed on 0.3 mmol scale in a flame
dried flask under argon. Enantiopurity was determined by
using chiral HPLC.

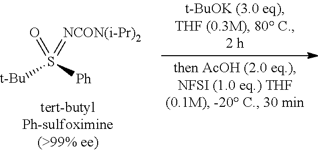

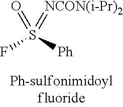

| | | Enantiopurity | Yield |
|---|---|---|---|
| Entry | Variations | (% ee) | (%)^{a} |
| 1 | None | > 99 | 81 |
| 2 | None | > 99 | 84^{b} |
| 3 | None | > 99 | 78^{c} |
| 4 | 2.5 eq. AcOH | >99 | 73 |
| 5 | 1.5 eq. AcOH | >99 | 75 |
| 6 | 0° C. insteand of −20° C. | 98 | 80 |
| 7 | no AcOH | NA^{d} | ND^{e} |
| 8 | Selectfluor (2.2 eq.), MeCN (0.1M) | 40 | 62 |
| | 50° C., 48 h | | |

ªIsolated yield.
^{b}reaction performed on 1.5 mmol scale (500 mg).
^{c}reaction performed on >3 mmol scale (>1 g).
^{d}Not available.
^{e}Not detected.

### General Procedure 6 (GP-6): One-Pot Transformation of N,N-Diisopropyl Urea Protected Chiral Tert-Butyl Sulfoximines to Sulfonimidoyl Fluorides.

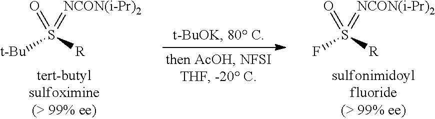

To a flame dried round bottom flask equipped with magnetic stir bar and argon balloon was added t-Bu sulfoximine (1.0 eq) and anhydrous THF (0.3 M). Once dissolved, solid anhydrous t-BuOK (3.0 eq) was added, and the reaction stirred at room temperature for 2-5 minutes. The argon balloon was removed, and the reaction placed in a pre-heated oil bath set to 80° C. for 2 hours (behind a blast shield). After 2 h (or upon completion; checked by TLC), the argon balloon was replaced then the reaction was cooled to −20° C. with dry ice and acetone bath. AcOH (2.0 eq) dissolved in anhydrous THF was added slowly to dilute the reaction to 0.1 M. Solid NSFI (1.0 eq) was added in one portion, and the reaction stirred at −20° C. for 30 mins. Upon completion (checked by TLC) the reaction was quenched with saturated aqueous NH₄Cl solution at −20° C. and extracted with EtOAc (×3). Combined organic layers were washed with water (×3) and brine (×3), dried over Na₂SO₄, filtered and concentrated under reduced pressure. Further purification was performed by silica gel column chromatography to provide the desired sulfonimidoyl fluoride.

Synthesis of Chiral Sulfoximines and Sulfonimidamides from N,N-Diisopropyl Urea Protected Sulfonimidoyl Fluorides.

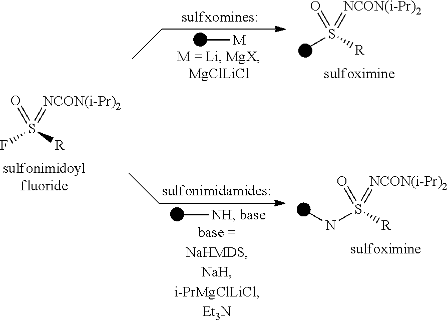

General Procedure 7 (GP-7): Synthesis of N,N-Diisopropyl Urea Protected Chiral Sulfoximines from Sulfonimidoyl Fluorides Via Grignard Reagents.

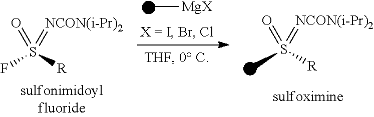

The Grignard Reagents were Used as Purchased or Prepared as Follows:

In a 10 mL flame dried round-bottomed flask equipped a stir bar and argon balloon was added Mg turnings (73 mg, 3 mmol, 1.5 eq.) and I₂ (approx. 5 mg, 0.02 mmol, 0.01 eq.) then vacuumed and refilled with argon. A portion of alkyl bromide or iodide (2 mmol, 1 eq.) solution in anhydrous THF (4 mL) was added with gentle heat until I₂ color disappeared, the rest of solution was then added dropwise.

After titration with iodine, the proper amount of the Grignard reagent (0.275 mmol, 1.1 eq.) was added dropwise to a solution of the sulfonimidoyl fluoride (0.25 mmol, 1.0 eq.) in THF (2.5 mL) in a separate 5 mL flame dried septum capped vial equipped a stir bar and argon balloon at 0° C. The reaction was stirred at 0° C. for 30 minutes then warmed to room temperature (unless otherwise stated below). Upon completion (checked by TLC) the reaction was quenched with saturated aqueous NH₄Cl (5 mL) then extracted with EtOAc (10 mL×3). The combined organic layers were washed with water (5 mL×3) then brine (5 mL×3), dried over anhydrous MgSO₄, filtered and concentrated under reduced pressure. Further purification by silica gel column chromatography provided the desired sulfoximines.

General Procedure 8 (GP-8): Synthesis of N,N-Diisopropyl Urea Protected Chiral Sulfoximines from Sulfonimidoyl Fluorides Via Turbo-Grignard Reagents.

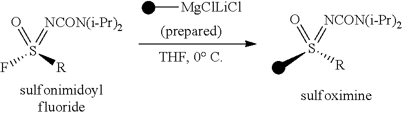

The preparation of turbo-Grignard reagents was modified from the Knochel methods, with slight variations depending on each substrate. In a 5 mL flame dried vial equipped with a stir bar and argon balloon was added isopropylmagnesium chloride lithium chloride (i-PrMgClLiCl) complex solution (0.21 mL, 0.275 mmol, 1.1 eq., 1.3 M in THF) followed by aryl bromide/iodide (0.275 mmol, 1.1 eq.) dissolved in dry THF (0.2 mL) at indicated temperature and exchange for indicated time (vide infra). Upon complete Mg-halogen exchange, a solution of sulfonimidoyl fluoride (0.25 mmol, 1 eq.) in THF (2.5 mL) was added dropwise at 0° C., stirred for 30 minutes then warmed to room temperature. Upon completion (checked by TLC) the reaction was quenched with saturated aqueous NH₄Cl (5 mL) then extracted with EtOAc (10 mL×3). The combined organic layers were washed with water (5 mL×3) then brine (5 mL×3), dried over anhydrous MgSO₄, filtered and concentrated under reduced pressure. Further purification by silica gel column chromatography provided the desired sulfoximines.

General Procedure 9 (GP-9): Synthesis of N,N-Diisopropyl Urea Protected Chiral Sulfonimidamides from Sulfonimidoyl Fluorides Using Li/Na/K-HMDS as a Base.

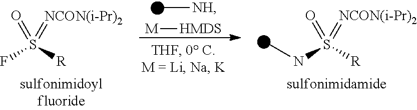

To a flame dried round bottom flask equipped with magnetic stir bar and argon balloon was sulfonimidoyl fluoride (0.25 mmol, 1.0 eq.) and amine (1.0 eq.) in anhydrous THF (0.1 M) at 0° C. M-HMDS (2.0 eq.) was added dropwise to the stirring mixture at 0° C. The reaction slowly warmed to room temperature where it stirred. Upon completion (checked by TLC) the reaction was quenched with silica gel then DCM was added, and solvent removed to adsorb the crude material to silica gel. Purification by column chromatography provided the desired sulfonimidamides.

General Procedure 10 (GP-10): Synthesis of N,N-Diisopropyl Urea Protected Chiral Sulfonimidamides from Sulfonimidoyl Fluorides Using Turbo-Amides.

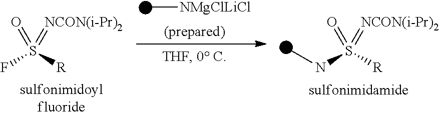

For aliphatic amines: To a flame dried round bottom flask equipped with magnetic stir bar and argon balloon was amine (2 eq. for 1° amines, 1 eq. for 2° amines) in anhydrous THF (0.1 M) at 0° C. i-PrMgCl—LiCl (2 eq. for 1° amines, 1:1; 1 eq. for 2° amines, 1:1) was added dropwise under 0° C. After 30 minutes of stirring, sulfonimidoyl fluoride (1.0 eq.) in THF (0.5 M) was added dropwise then warmed to room temperature. Upon completion (checked by TLC) the reaction was quenched with saturated aqueous NH₄Cl then extracted with EtOAc (×3). The combined organic layers were washed with water (×3) then brine (×3), dried over anhydrous MgSO₄, filtered and concentrated under reduced pressure. Further purification by silica gel column chromatography provided the desired sulfonimidamides.

For ammonium chloride or bromide: To a flame dried round bottom flask equipped with magnetic stir bar and argon balloon was ammonium chloride or bromide (3.0 eq.) in anhydrous THF (0.1 M) at 0° C. i-PrMgCl—LiCl (6.0 eq.) was added dropwise to the vigorously stirring mixture at 0° C. (cloudy mixture) then warmed to room temperature where the reaction mixture stirred until it became nearly clear (1 hour). The reaction mixture was cooled to 0° C. then sulfonimidoyl fluoride (1 eq.) in THF (0.5 M) was added dropwise and slowly warmed to room temperature. Upon completion (checked by TLC) the reaction was quenched with saturated aqueous NH₄Cl then extracted with EtOAc (×3). The combined organic layers were washed with water (×3) then brine (×3), dried over anhydrous MgSO₄, filtered and concentrated under reduced pressure. Further purification by silica gel column chromatography provided the desired sulfonimidamides.

General Procedure 11 (GP-11): Synthesis of N,N-Diisopropyl Urea Protected Chiral Sulfonimidamides from Sulfonimidoyl Fluorides Using Et₃N Under Thermal Conditions.

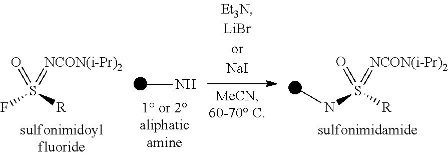

This reaction condition was adopted from the report by Bull and Luecking and slightly modified. In a flame dried septum capped vial equipped with a stir bar and argon balloon was added sulfonimidoyl fluoride (1 eq.) followed by MeCN (0.3 M). The amine (1-1.5 eq.), LiBr (2 eq.) or NaI (2 eq.), and Et₃N (2 eq.) were added. The argon balloon was removed, and the reaction was heated to 60-70° C. for the indicated reaction time (3-48 hours). Upon completion (checked by TLC and/or LC-MS) the solvent was removed under reduced pressure and the crude material purified by silica gel column chromatography.

General Procedure 12 (GP-12): Rapid Synthesis of N,N-Diisopropyl Urea Protected Chiral Sulfoximines and Sulfonimidamides from Tert-Butyl Sulfoximines in One-Pot.

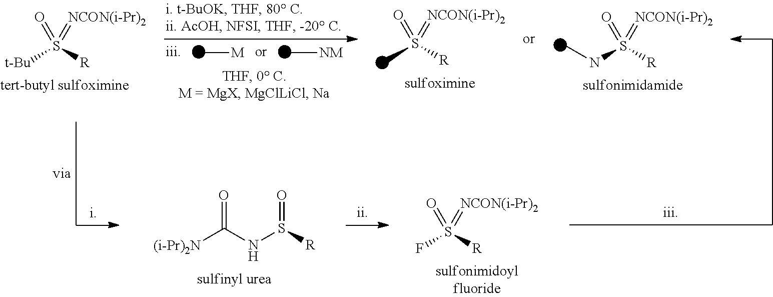

To a flame dried round-bottomed flask equipped with magnetic stir bar and argon balloon was added tert-butyl sulfoximine (1 eq.) followed by anhydrous THF (0.3 M). Once dissolved, solid anhydrous t-BuOK (3.0 eq) was added, and the reaction stirred at room temperature for 2-5 minutes. The argon balloon was removed, and the reaction placed in a pre-heated oil bath set to 80° C. for 2 hours (behind a blast shield). After 2 h (or upon completion; checked by TLC), the argon balloon was replaced then the reaction was cooled to −20° C. with dry ice and acetone bath. AcOH (2.0 eq) dissolved in anhydrous THF was added slowly to dilute the reaction to 0.1 M. Solid NSFI (1.0 eq) was added in one portion, and the reaction stirred at −20° C. for 30 mins. Upon completion (checked by TLC) either the Grignard or amine nucleophile was added.

### Grignard Nucleophiles:

The reaction mixture was cooled to −78° C. then the Grignard reagent (2 eq.) was added dropwise and stirred for one hour at −78° C. Upon completion (checked by TLC) the reaction was quenched with saturated aqueous NH₄Cl solution then extracted with EtOAc (×3). The combined organic layers were washed with water (×3) then brine (×3), dried over anhydrous Na₂SO₄, filtered and concentrated under reduced pressure. Further purification by silica gel column chromatography provided the desired sulfoximines.

### Amine Nucleophiles:

While the reaction was still at −20° C. from the fluorination step, the amine (1 eq.) and NaHMDS (2 eq.) were sequentially added to the reaction then slowly warmed to room temperature. Upon completion (checked by TLC) the reaction was quenched with silica gel then DCM was added, and solvent removed to adsorb the crude material to silica gel. Purification by column chromatography provided the desired sulfonimidamides.

Removal of N,N-Diisopropyl Urea-Type Groups from Sulfonimidoyl Compounds: A New Protecting Group for Sulfoximines and Sulfonimidamides.

### Optimization for the Removal of N,N-Diisopropyl Urea Protecting Group to Give N—H Sulfoximines.

TABLE 5
All reactions were performed on 0.1 mmol scale in a flame
dried flask or vial under argon.

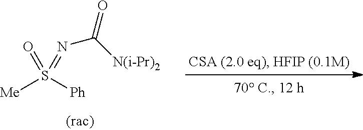

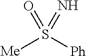

| | | Conversion | Yield |
|---|---|---|---|
| Entry | Variations | (%)^{a} | (%)^{b} |
| 1 | None | 100 | 93 |
| 2 | 60° C., 36 h | 100 | 93 |
| 3 | 4.0M HCl (aq., 8.0 eq.): | 100 | 87 |
| | AcOH = 1:1, 100° C., 12 h | | |
| 4 | 4.0M HCl (aq., 8.0 eq.): | 100 | 87 |
| | Dioxane = 1:1, 100° C., 12 h | | |
| 5 | PTSA (4.0 eq.), EtOH (0.1M) | <5 | NA^{c} |
| | 80° C., 20 h | | |
| 6 | MsOH (4.0 eq.), EtOH (0.1M) | <5 | NA^{c} |
| | 80° C., 20 h | | |
| 7 | TfOH (4.0 eq.), EtOH (0.1M) | <5 | NA^{c} |
| | 80° C., 20 h | | |
| 8 | LiOH (1.0M, aq, 3.0 eq.) | 0 | NA^{c} |
| | THF (0.1M), 80° C., 24 h | | |
| 9 | NH₄OH (aq., 30% w/w, 3.0 eq.) | 0 | NA^{c} |
| | Dioxane (0.1M), 100° C., 24 h | | |
| 10 | LiAlH₄ (3.0 eq.), THF (0.1M) | 64 | 57 |
| | r.t, 12 h | | |
| 11 | SmI₂-H₂O-LiBr (5:100:100 eq.) | 0 | NA^{c} |
| | THF (0.05 M), rt, 8 h | | |
| 12 | SmI₂-H₂O-Et₃N (6:72:72 eq.) | 0 | NA^{c} |
| | THF (0.05M), rt, 8 h | | |
| 13 | NaH (3.0 eq.), ZnI₂ (1.0 eq.) | 0 | NA^{c} |
| | NaI (1.0 eq.), THF (0.2M) | | |
| | 40° C., 12 h | | |
| 14 | (Ir(COE)₂Cl)₂ (5%), Et₂SiH₂ (4.0 eq.) | 0 | NA^{c} |
| | THF (0.3M), rt, 24 h | | |
| 15 | 9-BBN (2.0 eq.), THF (0.1M) | 0 | NA^{c} |
| | rt, 12 h | | |
| 16 | Ti(OiPr)₄ (1.0 eq.), PhSiH₃ (1.1 eq.) | 0 | NA^{c} |
| | THF (0.5M), rt, 3 h | | |
| 17 | DMSO/H₂O = 10:1(0.1M) | 0 | NA^{c} |
| | 80° C., 12 h^{d} | | |

ªDetermined by LC-MS.
^{b}Isolated yield. Not available.
^{c}This condition works for primary sulfonimidamides.

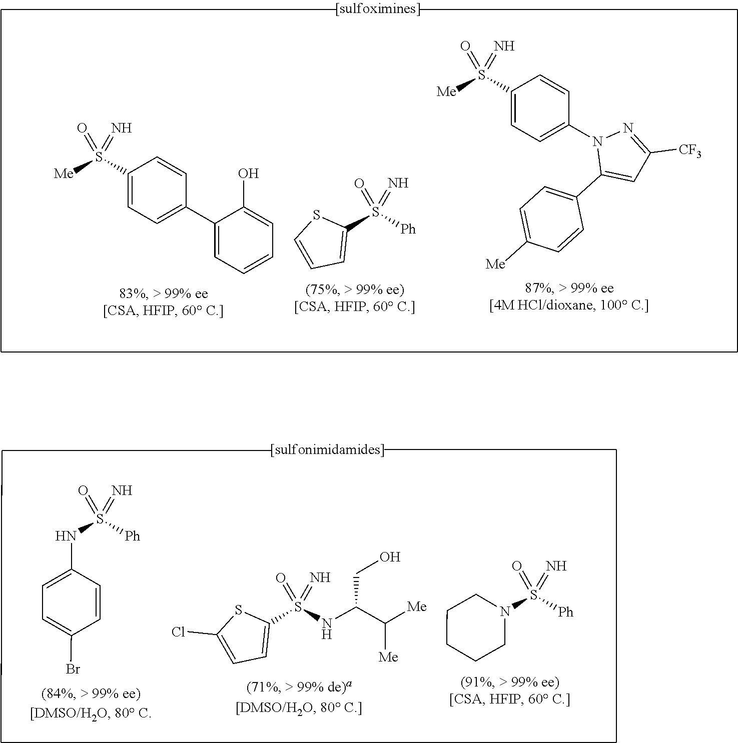

Examples representing the sulfoximine and sulfonimidamide scope for the deprotection of N,N-diisopropyl urea-type sulfonimidoyl protecting group. Enantiopurity was determined by using chiral HPLC. ªYield after deprotection of the sulfonimidoyl and alcohol (OTBS) protecting groups.

### General Procedure 13 (GP-13): Deprotection of N,N-Diisopropyl Urea Protected Chiral Sulfoximines and Sulfonimidamides.

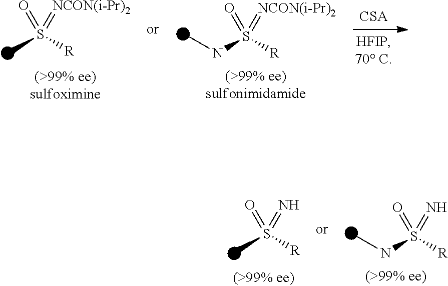

In a flask or vial equipped with a stir bar was added sulfoximine or sulfonimidamide (1 eq.) followed by CSA (2.0 eq) and HFIP (0.1 M). The reaction vessel was tightly capped then heated to 70° C. and stirred overnight (typically 12 h). Upon completion (checked by TLC) the reaction was cooled to room temperature then quenched with saturated aqueous NaHCO₃ to adjust the pH to neutral. The mixture was extracted with EtOAc (×4), washed with brine and purified by column chromatography to give the desired deprotected sulfoximine or sulfonimidamides.

### General Procedure 14 (GP-14): Specific Deprotection Conditions for N,N-Diisopropyl Urea Protected Chiral Primary Sulfonimidamides.

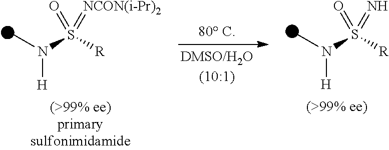

In a flask or vial equipped with a stir bar was added the primary sulfonimidamide (1 eq.) followed by DMSO/H₂O (10:1, 0.1 M) then heated to 80° C. (typically 8 hours). Upon completion (checked by TLC) the reaction was cooled to room temperature, diluted with water, and extracted with EtOAc (×4). The combined organic layers were washed with brine, dried over Na₂SO₄, filtered and concentrated. Further purification by silica gel column chromatography gave the desired deprotected products.

### Recrystallization of Tert-Butyl Sulfoximines to Further Enhance Enantiopurity.

For the cases in which organolithium additions to t-BuSF do not give the desired enantiopurity, recrystallizations can be performed to further enhance the enantiopurity of tert-butyl sulfoximines. We have found that nearly all N,N-diisopropyl urea protected chiral tert-butyl sulfoximines prepared from t-BuSF are solids at room temperature and have the potential to further enhance the enantiopurity if desired.

For example: tert-butyl phenyl sulfoximine was used as our model substrate to demonstrate the bifunctional property of the t-BuSF sulfonimidoyl transfer reagents. The addition of PhLi (commercial or in situ generated) to t-BuSF provides tert-butyl phenyl sulfoximine in 98% ee as a white solid which was enhanced to >99% ee via recrystallization.

The general procedure (GP-14) for typical recrystallizations is as follows: Pure tert-butyl sulfoximine was dissolved in a minimum amount of acetone, with the help of ultrasonic bath or heat. Hexanes (three times the volume of acetone used) was slowly added to prevent complete mixing of the two solvents. The mixture was transformed to −20° C. freezer to settle overnight to induce recrystallization. After 12 hours the recrystallized material was collected by filtration and washed with hexanes (×3) to give 60-70% recovery yield and >99% ee after a single recrystallization. This process can be repeated two more times to give up to 90% recovery yield with >99% ee.

### Other Examples

tert-butyl cyclopropyl sulfoximine (>90% recovery, 97 to >99% ee, three crops).

tert-butyl 4-chlorophenyl sulfoximine (>90% recovery, 95 to >99% ee; three crops).

First Functionalization of t-BuSF Reagent: Chiral Tert-Butyl Sulfoximine Product Characterization Data.

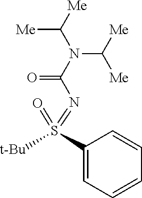 [SMILES: CC(C)N(C(=O)N=[S@](=O)(c1ccccc1)C(C)(C)C)C(C)C]

GP-1 and GP-2 were used with commercially available bromobenzene (0.375 mmol, 1.5 eq.) with no further modifications. Purified by silica gel column chromatography using hexane/EtOAc (0% to 50% EtOAc gradient) to give the product (70 mg, 217 μmol, 87% yield) as a white crystalline solid.

There were no significant differences in yield or enantiopurity between the two procedures. Phenyl lithium can be used instead of generating phenyl lithium from n-BuLi or t-BuLi with no decrease in yield or enantiopurity.

### Different Scale Reactions:

GP-1 was followed with commercial PhLi (5.33 mL, 10.1 mmol, 1.9 M, dibutylether, 1.5 eq.) and t-BuSF (1.80 g, 6.76 mmol, 1 eq.), produced (1.77 g, 5.45 mmol, 81% yield, 98% ee).

GP-1 was followed with commercial PhLi (8.89 mL, 16.9 mmol, 1.9 M, dibutylether, 1.5 eq.) and t-BuSF (3.0 g, 16.9 mmol, 1 eq.), produced (3.30 g, 10.2 mmol, 90% yield, 98% ee).

Physical characteristics: White crystalline solid. TLC: R_{f}=0.27 (hexane/EtOAc, 50% EtOAc, UV). ¹H NMR: (500 MHZ, CDCl₃) δ 7.84-7.73 (m, 2H), 7.61-7.56 (m, 1H), 7.55-7.50 (m, 2H), 4.06 (s, 2H), 1.37 (s, 9H), 1.27 (d, J=8.3 Hz, 12H) ppm. ¹³C NMR: (126 MHz, CDCl₃) δ 159.55, 135.23, 132.89, 130.21, 128.92, 60.78, 45.96, 23.75, 21.39 ppm. Specific rotation:

$$[ α ] \frac{23}{D} = + 28.85$$

(c 0.50, CHCl₃) HRMS: Calc'd for C₁₇H₂₉N₂O₂S [M+H⁺] 325.1944; found 325.1951. Melting Point: 177-178° C. Enantiomeric excess: 98% ee. Recrystallized to >99% ee with >90% recovery. HPLC Conditions: Daicel Chiralpak IC column, 70:30 n-hexane:i-PrOH, flow rate: 1 mL min-1, 25° C., UV detection wavelength: 220 nm, retention time: major: 17.127 min, minor: 26.107 min.

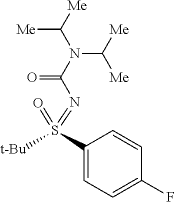 [SMILES: CC(C)N(C(=O)N=[S@](=O)(c1ccc(F)cc1)C(C)(C)C)C(C)C]

GP-1 was followed with no additional modifications: Commercially available 1-bromo-4-fluorobenzene (0.375 mmol, 1.5 eq.) was used. Purified by silica gel column chromatography using hexane/EtOAc (0% to 40% EtOAc gradient) to give the product (71 mg, 207 μmol, 83% yield) as a white amorphous solid.

Physical characteristics: White amorphous solid. TLC: R_{f}=0.4 (hexane/EtOAc, 30% EtOAc, UV). ¹H NMR: (500 MHz, CDCl₃) δ 7.84-7.72 (m, 2H), 7.21 (dd, J=9.1, 8.2 Hz, 2H), 4.05 (s, 2H), 1.37 (s, 9H), 1.34-1.18 (m, 12H) ppm. ¹³C NMR: 126 MHz, CDCl₃) δ 165.62 (d, J=254.8 Hz), 159.38, 132.74 (d, J=9.4 Hz), 131.06 (d, J=3.2 Hz), 116.32 (d, J=22.6 Hz), 60.92, 45.97, 23.71, 21.36 ppm. 19F NMR: (471 MHz, CDCl₃) δ −105.86 ppm. Specific rotation:

$$[ α ] \frac{23}{D} = - 15.24$$

(c 1.00, CHCl₃) HRMS: Calc'd for C₁₇H₂₇FN₂NaO₂S [M+Na⁺] 365.1669; found 365.1666. Enantiomeric excess: 97% ee. HPLC Conditions: Daicel Chiralpak IC column, 70:30 n-hexane:i-PrOH, flow rate: 1 mL min-1, 25° C., UV detection wavelength: 220 nm, retention time: minor: 13.733 min, major: 17.120 min.

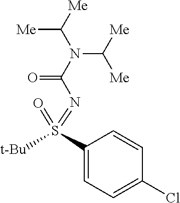 [SMILES: CC(C)N(C(=O)N=[S@](=O)(c1ccc(Cl)cc1)C(C)(C)C)C(C)C]

GP-1 was followed with no additional modifications: Commercially available 1-bromo-4-chlorobenzene (0.375 mmol, 1.5 eq.) was used. Purified by silica gel column chromatography using hexane/EtOAc (0% to 50% EtOAc gradient) to give the product (75 mg, 208 μmol, 85% yield) as a white crystalline solid.

GP-2 was also followed and showed no significant difference on yield or enantiopurity.

Physical characteristics: White crystalline solid. TLC: R_{f}=0.39 (hexane/EtOAc, 30% EtOAc, UV). ¹H NMR: (500 MHZ, CDCl₃) δ 7.70 (d, J=8.6 Hz, 2H), 7.50 (d, J=8.8 Hz, 2H), 4.06 (d, 2H), 1.37 (s, 21H) ppm. ¹³C NMR: (126 MHZ, CDCl₃) δ 159.38, 139.71, 133.96, 131.55, 129.33, 60.92, 46.47, 45.37, 23.72, 21.88, 20.85 ppm. Specific rotation:

$$[ α ] \frac{23}{D} = - 5.53$$

(c 1.00, CHCl₃) HRMS: Calc'd for C₁₇H₂₈ClN₂O₂S [M+H⁺] 359.1555; found 359.1560. Melting Point: 178-180° C. Enantiomeric excess: 95% ee. HPLC Conditions: Daicel Chiralpak IC column, 70:30 n-hexane:i-PrOH, flow rate: 1 mL min-1, 25° C., UV detection wavelength: 220 nm, retention time: major: 15.767 min, minor: 18.047 min.

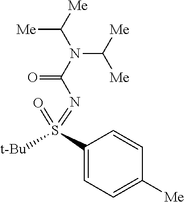 [SMILES: Cc1ccc([S@@](=O)(=NC(=O)N(C(C)C)C(C)C)C(C)(C)C)cc1]

GP-1 was followed with no additional modifications: Commercially available 4-bromotoluene (0.375 mmol, 1.5 eq.) was used. Purified by silica gel column chromatography using hexane/EtOAc (0% to 50% EtOAc gradient) to give the product (71 mg, 209 μmol, 84% yield) as a white amorphous solid.

GP-2 was also followed and showed no significant difference on yield or enantiopurity.

Physical characteristics: White amorphous solid. TLC: R_{f}=0.27 (hexane/EtOAc, 33% EtOAc). ¹H NMR: (500 MHz, CDCl₃) δ 7.67 (d, 2H), 7.33 (d, 2H), 4.12 (d, J=31.4 Hz, 2H), 2.44 (s, 3H), 1.39 (s, 15H), 1.20 (d, 6H) ppm. ¹³C NMR: (126 MHZ, CDCl₃) δ 159.66, 143.65, 132.06, 130.21, 129.68, 60.67, 46.40, 45.17, 23.74, 21.86, 21.66, 20.88 ppm. Specific rotation:

$$[ α ] \frac{23}{D} = - 9.12$$

(c 1.00, CHCl₃) HRMS. Calc'd for C₁₈H₃₁N₂O₂S [M+H⁺] 339.2101; found 339.2101. Enantiomeric excess: 97% ee. HPLC Conditions: Daicel Chiralpak IC column, 70:30 n-hexane:i-PrOH, flow rate: 1 mL min-1, 25° C., UV detection wavelength: 220 nm, retention time: major: 27.513 min, minor: 36.560 min.

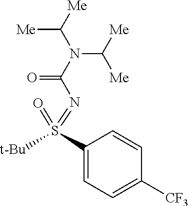 [SMILES: CC(C)N(C(=O)N=[S@](=O)(c1ccc(C(F)(F)F)cc1)C(C)(C)C)C(C)C]

GP-1 was followed with no additional modifications: Commercially available 1-bromo-4-(trifluoromethyl)benzene (0.375 mmol, 1.5 eq.) was used. Purified by silica gel column chromatography using hexane/EtOAc (0% to 40% EtOAc gradient) to give the product (71.6 mg, 183 μmol, 73% yield) as a white amorphous solid.

Physical characteristics: White amorphous solid. TLC: R_{f}=0.27 (hexane/EtOAc, 25% EtOAc). ¹H NMR: (500 MHZ, CDCl₃) δ 7.91 (d, 2H), 7.79 (d, 2H), 4.08 (d, 2H), 1.39 (s, 9H), 1.36 (s, 6H), 1.16 (dd, J=23.0, 6.7 Hz, 6H) ppm. ¹³C NMR: (126 MHZ, CDCl₃) δ 159.31, 139.61, 134.59 (q, J=32.8 Hz), 130.69, 126.04 (q, J=3.7 Hz), 123.50 (q, J=273.0 Hz), 61.03, 46.56, 45.49, 24.73, 23.74, 21.84, 20.90, 20.79 ppm. 19F NMR: (471 MHZ, CDCl₃) δ −63.07 ppm. Specific rotation:

$$[ α ] \frac{23}{D} = - 8.76$$

(c 1.00, CHCl₃) HRMS: Calc'd for C₁₈H₂₈F₃N₂O₂S [M+H⁺] 393.1818; found 393.1824. Enantiomeric excess: 95% ee. HPLC Conditions: Daicel Chiralpak IC column, 70:30 n-hexane:i-PrOH, flow rate: 1 mL min-1, 25° C., UV detection wavelength: 220 nm, retention time: major: 8.340 min, minor: 9.387 min.

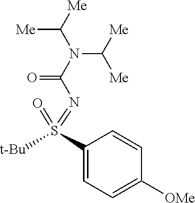 [SMILES: COc1ccc([S@@](=O)(=NC(=O)N(C(C)C)C(C)C)C(C)(C)C)cc1]

GP-1 was followed with no additional modifications: Commercially available 4-bromoanisole (0.375 mmol, 1.5 eq.) was used. Purified by silica gel column chromatography using hexane/EtOAc (0% to 40% EtOAc gradient) to give the product (72.6 mg, 206 μmol, 82% yield) as a white amorphous solid.

Physical characteristics: White amorphous solid. TLC: R_{f}=0.25 (hexane/EtOAc, 33% EtOAc). ¹H NMR: (500 MHZ, CDCl₃) δ 7.69 (d, J=8.8 Hz, 2H), 6.99 (d, J=9.1 Hz, 2H), 4.30-3.90 (m, 2H), 3.86 (s, 3H), 1.42-1.08 (m, 21H) ppm. ¹³C NMR: (126 MHZ, CDCl₃) δ 163.29, 159.63, 132.14, 126.22, 114.36, 60.82, 55.70, 45.24, 23.74, 21.83, 21.13 ppm. Specific rotation:

$$[ α ] \frac{23}{D} = - 1.51$$

(c 1.00, CHCl₃) HRMS: Calc'd for C₁₈H₃₁N₂O₃S [M+H⁺] 355.2050; found 355.2051. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 40.207 min, minor: 46.987 min.

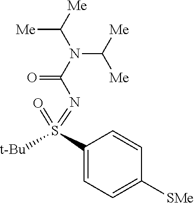 [SMILES: CSc1ccc([S@@](=O)(=NC(=O)N(C(C)C)C(C)C)C(C)(C)C)cc1]

GP-1 was followed with no additional modifications: Commercially available 4-bromothioanisole (0.375 mmol, 1.5 eq.) was used. Purified by silica gel column chromatography using hexane/EtOAc (0% to 50% EtOAc gradient) to give the product (69 mg, 187 μmol, 75% yield) as a white amorphous solid.

Physical characteristics: White amorphous solid. TLC: R_{f}=0.19 (hexane/EtOAc, 33% EtOAc). ¹H NMR: (500 MHz, CDCl₃) δ 7.64 (d, J=8.6 Hz, 2H), 7.31 (d, J=8.8 Hz, 2H), 4.06 (s, 2H), 2.51 (s, 3H), 1.37 (s, 9H), 1.25 (t, J=8.0 Hz, 12H) ppm. ¹³C NMR: ¹³C NMR (126 MHz, CDCl₃) δ 159.54, 146.02, 130.75, 130.40, 125.43, 60.91, 46.01, 23.72, 21.33,

$$[ α ] \frac{23}{D} = + 10.1$$

(c 1.00, CHCl₃) HRMS: Calc'd for C₁₈H₃₁N₂O₂S₂ [M+H⁺] 371.1821; found 371.1823. Enantiomeric excess: 98% ee. HPLC Conditions: Daicel Chiralpak IC column, 70:30 n-hexane:i-PrOH, flow rate: 1 mL min-1, 25° C., UV detection wavelength: 220 nm, retention time: major: 40.480 min, minor: 45.533 min.

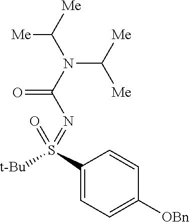 [SMILES: CC(C)N(C(=O)N=[S@](=O)(c1ccc(OCc2ccccc2)cc1)C(C)(C)C)C(C)C]

GP-1 was followed with no additional modifications: Commercially available 1-(benzyloxy)-4-bromobenzene (0.375 mmol, 1.5 eq.) was used. Purified by silica gel column chromatography using hexane/EtOAc (0% to 40% EtOAc gradient) to give the product (43 mg, 102 μmol, 40% yield) as a white amorphous solid.

Physical characteristics: White amorphous TLC: R_{f}=0.25 (hexane/EtOAc, 30% EtOAc). ¹H NMR: (500 MHz, CDCl₃) δ 7.69 (d, J=8.5 Hz, 2H), 7.44-7.32 (m, 5H), 7.10-7.03 (m, 2H), 5.13-5.05 (m, 2H), 4.07 (d, J=44.0 Hz, 2H), 1.42-1.27 (m, 15H), 1.25-1.09 (m, 6H) ppm. ¹³C NMR: (126 MHZ, CDCl₃) δ 162.55, 159.64, 136.14, 132.19, 128.87, 128.48, 127.76, 126.51, 115.14, 70.54, 60.84, 45.45, 23.75, 21.95, 20.86 ppm. Specific rotation:

$$[ α ] \frac{23}{D} = + 47.35$$

(c 0.50, CHCl₃) HRMS: Calc'd for C₂₄H₃₅N₂O₃S [M+H⁺] 431.2363; found 431.2359. Enantiomeric excess: 97% ee. HPLC Conditions: Daicel Chiralpak IC column, 70:30 n-hexane:i-PrOH, flow rate: 1 mL min-1, 25° C., UV detection wavelength: 220 nm, retention time: major: 36.433 min, minor: 52.920 min.

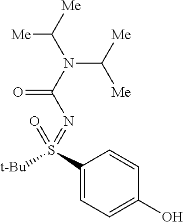 [SMILES: CC(C)N(C(=O)N=[S@](=O)(c1ccc(O)cc1)C(C)(C)C)C(C)C]

GP-1 was followed with no additional modifications: Commercially available 4-bromophenol (or with −TBS protection) were used. Purified by silica gel column chromatography using DCM/MeOH (0% to 10% MeOH gradient) to give the product (63.9 mg, 188 μmol, 75% yield) as a white amorphous solid.

Note: The OTBS protected phenol gave similar yield and exact enantiopurity. The silyl protecting group is removed during the reaction.

Physical characteristics: White amorphous solid. TLC: R_{f}=0.20 (DCM/MeOH, 10% MeOH). ¹H NMR: (500 MHz, CDCl₃) δ 9.54 (s, 1H), 7.36 (d, J=8.3 Hz, 2H), 6.66-6.59 (m, 2H), 4.12 (d, J=121.9 Hz, 2H), 1.42 (d, J=6.8 Hz, 6H), 1.33 (s, 9H), 1.21 (dd, J=14.0, 6.8 Hz, 6H) ppm. ¹³C NMR: ¹³C NMR (126 MHz, CDCl₃) δ 162.28, 161.13, 131.79, 122.22, 116.53, 61.15, 46.16, 45.95, 23.55, 21.87, 21.72, 20.99, 20.87 ppm. Specific rotation:

$$[ α ] \frac{23}{D} = - 66.29$$

(c 1.00, CHCl₃) HRMS: Calc'd for C₁₇H₂₈N₂NaO₃S [M+Na⁺] 363.1713; found 363.1705. Enantiomeric excess: 97% ee. HPLC Conditions: (hydroxy group was methylated for HPLC analysis) Daicel Chiralpak IC column, 70:30 n-hexane:i-PrOH, flow rate: 1 mL min-1, 25° C., UV detection wavelength: 220 nm, retention time: major: 36.540 min, minor: 43.720 min.

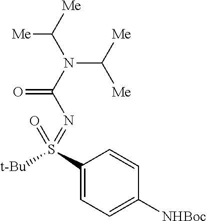 [SMILES: CC(C)N(C(=O)N=[S@](=O)(c1ccc(NC(=O)OC(C)(C)C)cc1)C(C)(C)C)C(C)C]

GP-1 was followed with additional modifications mentioned: Commercially available tert-butyl (4-bromophenyl) carbamate (0.375 mmol, 1.5 eq.) was used and equivalents of n-BuLi was increased (from 1.5 to 3 eq.). Purified by silica gel column chromatography using hexane/Acetone (0% to 30% Acetone gradient) to give the product (65.9 mg, 150 μmol, 60% yield) as a white amorphous solid.

Physical characteristics: White amorphous solid. TLC: R_{f}=0.35 (hexane/Acetone, 30% Acetone). ¹H NMR: (500 MHZ, CDCl₃) δ 7.64 (d, 2H), 7.50 (d, 2H), 6.95 (s, 1H), 4.02 (d, 2H), 1.52 (s, 9H), 1.35 (s, 15H), 1.19 (d, 6H) ppm. ¹³C NMR: (126 MHZ, CDCl₃) δ 159.61, 152.43, 143.05, 131.37, 127.94, 118.11, 81.40, 60.88, 46.57, 45.31, 28.40, 23.71, 21.82, 21.01 ppm. Specific rotation:

$$[ α ] \frac{23}{D} = - 15.31$$

(c 1.00, CHCl₃) HRMS: Calc'd for C₂₂H₃₈N₃O₄S [M+H⁺] 440.2578; found 440.2574. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 9.673 min, minor: 13.367 min.

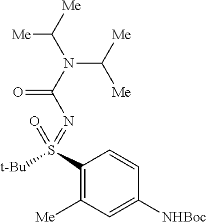 [SMILES: Cc1cc(NC(=O)OC(C)(C)C)ccc1[S@@](=O)(=NC(=O)N(C(C)C)C(C)C)C(C)(C)C]

GP-1 was followed with no additional modifications: Commercially available tert-butyl (4-bromo-3-methylphenyl) carbamate (0.375 mmol, 1.5 eq.) was used. n-BuLi was used with double amount: deprotonation and then exchange. Purified by silica gel column chromatography using hexane/Acetone (0% to 30% Acetone gradient) to give the product (75.7 mg, 167 μmol, 67% yield) as a white amorphous solid.

Physical characteristics: White amorphous solid. TLC: R_{f}=0.25 (hexane/Acetone, 30% Acetone). ¹H NMR: (500 MHz, CDCl₃) δ 7.79 (s, 1H), 7.47 (s, 1H), 7.15 (d, J=8.4 Hz, 1H), 6.71 (s, 1H), 4.35-3.79 (m, 2H), 2.58 (s, 3H), 1.49 (s, 9H), 1.38 (s, 9H), 1.34-1.16 (m, 12H) ppm. ¹³C NMR: (126 MHz, CDCl₃) δ 159.56, 152.54, 136.86, 133.60, 123.14, 122.68, 80.58, 62.33, 46.75, 45.08, 28.31, 23.72, 21.71, 21.52, 20.90, 20.76, 20.34 ppm. Specific rotation:

$$[ α ] \frac{23}{D} = - 33.81$$

(c 1.00, CHCl₃) HRMS: Calc'd for C₂₃H₃₉N₃NaO₄S [M+Na⁺] 476.2553; found 476.2549. Enantiomeric excess: 95% ee. HPLC Conditions: Daicel Chiralpak IB column, 95:05 n-hexane:i-PrOH, flow rate: 1 mL min-1, 25° C., UV detection wavelength: 220 nm, retention time: major: 5.847 min, minor: 7.053 min.

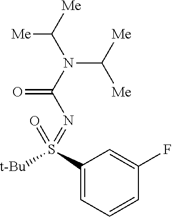 [SMILES: CC(C)N(C(=O)N=[S@](=O)(c1cccc(F)c1)C(C)(C)C)C(C)C]

GP-1 was followed with no additional modifications: Commercially available 1-bromo-3-fluorobenzene (0.375 mmol, 1.5 eq.) was used. Purified by silica gel column chromatography using hexane/EtOAc (0% to 40% EtOAc gradient) to give the product (70.1 mg, 205 μmol, 82% yield) as a white solid.

Physical characteristics: White amorphous solid. TLC: R_{f}=0.25 (hexane/EtOAc, 30% EtOAc). ¹H NMR: (500 MHz, CDCl₃) δ 7.59-7.44 (m, 3H), 7.29 (tdd, J=8.2, 2.6, 1.1 Hz, 1H), 4.05 (s, 2H), 1.38 (s, 9H), 1.33-1.13 (m, 12H) ppm. ¹³C NMR: (126 MHZ, CDCl₃) δ 162.60 (d, J=251.2 Hz), 159.34, 137.81 (d, J=6.3 Hz), 130.51 (d, J=7.6 Hz), 125.97 (d, J=3.2 Hz), 120.17 (d, J=21.2 Hz), 117.54 (d, J=24.2 Hz), 61.04, 46.62, 45.39, 23.78, 21.81, 20.89, 20.81 ppm. 19F NMR: (471 MHz, CDCl₃) δ −110.39 ppm. Specific rotation:

$$[ α ] \frac{23}{D} = - 13.65$$

(c 1.00, CHCl₃) HRMS: Calc'd for C₁₇H₂₈FN₂O₂S [M+H⁺] 343.1850; found 343.1853. Enantiomeric excess: 95% ee. HPLC Conditions: Daicel Chiralpak IC column, 70:30 n-hexane:i-PrOH, flow rate: 1 mL min-1, 25° C., UV detection wavelength: 220 nm, retention time: major: 12.113 min, minor: 13.687 min.

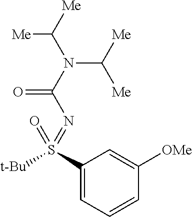 [SMILES: COc1cccc([S@@](=O)(=NC(=O)N(C(C)C)C(C)C)C(C)(C)C)c1]

GP-1 was followed with no additional modifications: Commercially available 3-bromoanisole (0.375 mmol, 1.5 eq.) was used. Purified by silica gel column chromatography using hexane/EtOAc (0% to 40% EtOAc gradient) to give the product (71.7 mg, 202 μmol, 81% yield) as a white solid.

Physical characteristics: White amorphous solid. TLC: R_{f}=0.20 (hexane/EtOAc, 30% EtOAc). ¹H NMR: (500 MHZ, CDCl₃) δ 7.42 (t, J=8.0 Hz, 1H), 7.36-7.30 (m, 2H), 7.11 (ddd, J=8.2, 2.5, 1.0 Hz, 1H), 4.30-3.88 (m, 2H), 3.83 (s, 3H), 1.38 (s, 9H), 1.36-1.06 (m, 12H) ppm. ¹³C NMR: (126 MHZ, CDCl₃) δ 159.93, 159.59, 136.52, 129.79, 122.36, 119.40, 115.01, 60.90, 55.68, 46.35, 45.38, 23.82, 21.82, 21.04 ppm. Specific rotation:

$$[ α ] \frac{23}{D} = - 1.76$$

(c 1.00, CHCl₃) HRMS: Calc'd for C₁₈H₂₇F₃N₂O₂S [M+H⁺] 355.2050; found 355.2051. Enantiomeric excess: 97% ee. HPLC Conditions: Daicel Chiralpak IC column, 70:30 n-hexane:i-PrOH, flow rate: 1 mL min-1, 25° C., UV detection wavelength: 220 nm, retention time: major: 23.827 min, minor: 30.987 min.

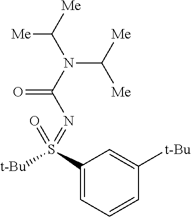 [SMILES: CC(C)N(C(=O)N=[S@](=O)(c1cccc(C(C)(C)C)c1)C(C)(C)C)C(C)C]

GP-1 was followed with no additional modifications: commercially available 1-bromo-3-(tert-butyl)benzene (0.375 mmol, 1.5 eq.) was used. Purified by silica gel column chromatography using hexane/EtOAc (0% to 40% EtOAc gradient) to give the product (68.4 mg, 180 μmol, 72% yield) as a white solid.

Physical characteristics: White amorphous solid. TLC: R_{f}=0.20 (hexane/EtOAc, 30% EtOAc). ¹H NMR: (500 MHZ, CDCl₃) δ 3.96 (s, 1H), 4.19 (s, 1H), 1.32 (s, 9H), 7.76 (t, J=2.0 Hz, 1H), 7.62-7.57 (m, 2H), 7.45 (t, J=7.8 Hz, 1H), 1.35 (s, 15H), 1.16 (dd, J=21.0, 7.0 Hz, 6H) ppm. ¹³C NMR: (126 MHZ, CDCl₃) δ 159.62, 151.98, 134.54, 129.96, 128.69, 127.55, 127.14, 60.63, 46.52, 45.22, 35.04, 31.28, 23.71, 21.80, 20.98, 20.90 ppm. Specific rotation:

$$[ α ] \frac{23}{D} = - 14.47$$

(c 1.00, CHCl₃) HRMS: Calc'd for C₂₁H₃₇N₂O₂S [M+H⁺] 381.2570; found 381.2572. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 9.720 min, minor: 14.593 min.

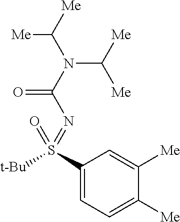 [SMILES: Cc1ccc([S@@](=O)(=NC(=O)N(C(C)C)C(C)C)C(C)(C)C)cc1C]

GP-1 was followed with no additional modifications: Commercially available 4-bromo-1,2-dimethylbenzene was used. Purified by silica gel column chromatography using hexane/EtOAc (0% to 40% EtOAc gradient) to give the product (73.3 mg, 207 μmol, 83% yield) as a white amorphous solid.

Physical characteristics: White amorphous solid. TLC: R_{f}=0.15 (hexane/EtOAc, 30% EtOAc). ¹H NMR: (500 MHz, CDCl₃) δ 7.55 (d, J=2.0 Hz, 1H), 7.45 (dd, J=8.0, 2.0 Hz, 1H), 7.26 (d, 1H), 4.13 (s, 1H), 3.99 (s, 1H), 2.30 (t, J=1.1 Hz, 6H), 1.37 (s, 15H), 1.16 (dd, J=25.2, 6.8 Hz, 6H) ppm. ¹³C NMR: (126 MHz, CDCl₃) δ 159.76, 142.38, 137.61, 132.12, 131.20, 130.17, 127.64, 60.62, 46.44, 45.21, 23.78, 21.84, 21.00, 20.87, 20.12, 20.04 ppm. Specific rotation:

$$[ α ] \frac{23}{D} = + 1.08$$

(c 1.00, CHCl₃) HRMS: Calc'd for C₁₉H₃₃N₂O₂S [M+H⁺] 353.2257; found 353.2260. Enantiomeric excess: 98% ee. HPLC Conditions: Daicel Chiralpak IC column, 70:30 n-hexane:i-PrOH, flow rate: 1 mL min-1, 25° C., UV detection wavelength: 220 nm, retention time: major: 30.753 min, minor: 43.480 min.

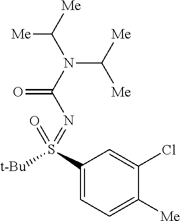 [SMILES: Cc1ccc([S@@](=O)(=NC(=O)N(C(C)C)C(C)C)C(C)(C)C)cc1Cl]

GP-1 was followed with no additional modifications: commercially available 4-bromo-1,2-dimethylbenzene (0.375 mmol, 1.5 eq.) was used. Purified by silica gel column chromatography using hexane/EtOAc (0% to 40% EtOAc gradient) to give the product (71.6 mg, 192 μmol, 77% yield) as a white amorphous solid.

Physical characteristics: White amorphous solid. TLC: R_{f}=0.20 (hexane/EtOAc, 30% EtOAc). ¹H NMR: (500 MHz, CDCl₃) δ 7.74 (d, J=1.9 Hz, 1H), 7.53 (dd, J=8.0, 1.9 Hz, 1H), 7.38 (dd, J=8.0, 0.9 Hz, 1H), 4.26-4.05 (m, 1H), 3.97 (s, 1H), 2.43 (s, 3H), 1.38 (s, 9H), 1.37-1.30 (m, 6H), 1.23-1.11 (m, 6H) ppm. ¹³C NMR: (126 MHZ, CDCl₃) δ 159.44, 141.76, 135.38, 134.28, 131.36, 130.65, 128.25, 60.96, 46.65, 45.37, 23.76, 21.80, 20.94, 20.82, 20.42 ppm. Specific rotation:

$$[ α ] \frac{23}{D} = - 2.06$$

(c 1.00, CHCl₃) HRMS: Calc'd for C₁₈H₃₀ClN₂O₂S [M+H⁺] 373.1711; found 373.1714. Enantiomeric excess: 98% ee. HPLC Conditions: Daicel Chiralpak IC column, 70:30 n-hexane:i-PrOH, flow rate: 1 mL min-1, 25° C., UV detection wavelength: 220 nm, retention time: major: 18.407 min, minor: 20.020 min.

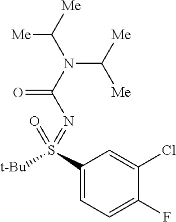 [SMILES: CC(C)N(C(=O)N=[S@](=O)(c1ccc(F)c(Cl)c1)C(C)(C)C)C(C)C]

GP-1 was followed with no additional modifications: Commercially available 4-bromo-1-chloro-2-fluorobenzene (0.375 mmol, 1.5 eq.) was used. Purified by silica gel column chromatography using hexane/EtOAc (0% to 40% EtOAc gradient) to give the product (71.5 mg, 190 μmol, 76% yield) as a white amorphous solid.

Physical characteristics: White amorphous solid. TLC: R_{f}=0.30 (hexane/EtOAc, 30% EtOAc). ¹H NMR: (500 MHz, CDCl₃) δ 7.84 (dd, J=6.7, 2.3 Hz, 1H), 7.64 (ddd, J=8.7, 4.3, 2.3 Hz, 1H), 7.29 (t, J=8.5 Hz, 1H), 4.04 (d, J=96.8 Hz, 2H), 1.39 (s, 9H), 1.33 (s, 6H), 1.17 (dd, J=24.9, 6.7 Hz, 6H) ppm. ¹³C NMR: (126 MHz, CDCl₃) δ 162.04, 159.58 (d, J=104.2 Hz), 132.93, 132.42 (d, J=4.0 Hz), 130.42 (d, J=8.3 Hz), 122.57 (d, J=18.7 Hz), 117.27 (d, J=22.5 Hz), 61.19, 46.72, 45.43, 23.73, 21.75, 20.87, 20.83 ppm. 19F NMR: (471 MHz, CDCl₃) δ −107.84 ppm. Specific rotation:

$$[ α ] \frac{23}{D} = - 5.67$$

(c 1.00, CHCl₃) HRMS: Calc'd for C₁₇H₂₇ClFN₂O₂S [M+H⁺] 377.1460; found 377.1454. Enantiomeric excess: 96% ee. HPLC Conditions: Daicel Chiralpak IA column, 95:05 n-hexane:i-PrOH, flow rate: 1 mL min-1, 25° C., UV detection wavelength: 220 nm, retention time: minor: 11.120 min, major: 14.553 min.

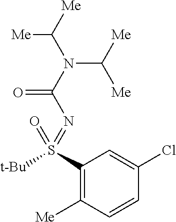 [SMILES: Cc1ccc(Cl)cc1[S@@](=O)(=NC(=O)N(C(C)C)C(C)C)C(C)(C)C]

GP-1 was followed with no additional modifications: Commercially available 2-bromo-4-chlorotoluene (0.375 mmol, 1.5 eq.) was used. Purified by silica gel column chromatography using hexane/EtOAc (0% to 40% EtOAc gradient) to give the product (67.2 mg, 180 μmol, 72% yield) as a white amorphous solid.

Physical characteristics: White amorphous solid. TLC: R_{f}=0.25 (hexane/EtOAc, 30% EtOAc). ¹H NMR: (500 MHz, CDCl₃) δ 7.81 (s, 1H), 7.39 (dd, J=8.2, 2.3 Hz, 1H), 7.30-7.18 (m, 1H), 4.06 (d, J=159.9 Hz, 2H), 2.63 (d, J=9.5 Hz, 3H), 1.40 (d, J=9.4 Hz, 9H), 1.35-1.14 (m, 12H) ppm. ¹³C NMR: (126 MHz, CDCl₃) δ 159.39, 135.30, 134.51, 132.63, 132.13, 62.48, 46.85, 45.22, 23.68, 21.58, 21.25-20.22 (m) ppm. Specific rotation:

$$[ α ] \frac{23}{D} = - 36.4$$

(c 1.00, CHCl₃) HRMS: Calc'd for C₁₈H₃₀ClN₂O₂S [M+H⁺] 373.1711; found 373.1710. Enantiomeric excess: 98% ee. HPLC Conditions: Daicel Chiralpak IC column, 70:30 n-hexane:i-PrOH, flow rate: 1 mL min-1, 25° C., UV detection wavelength: 220 nm, retention time: minor: 8.587 min, major: 11.913 min.

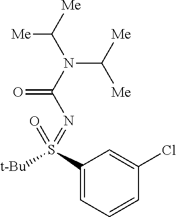 [SMILES: CC(C)N(C(=O)N=[S@](=O)(c1cccc(Cl)c1)C(C)(C)C)C(C)C]

GP-1 was followed with no additional modifications: Commercially available 1-bromo-3-chlorobenzene (0.375 mmol, 1.5 eq.) was used. Purified by silica gel column chromatography using hexane/EtOAc (0% to 40% EtOAc gradient) to give the product (74.3 mg, 207 μmol, 83% yield) as a white amorphous solid.

Physical characteristics: White amorphous solid. TLC: R_{f}=0.30 (hexane/EtOAc, 30% EtOAc). ¹H NMR: (500 MHz, CDCl₃) δ 7.76 (s, 1H), 7.64 (dt, J=8.0, 1.3 Hz, 1H), 7.55 (ddd, J=8.0, 2.1, 1.1 Hz, 1H), 7.47 (t, J=7.9 Hz, 1H), 4.29-3.86 (m, 2H), 1.38 (s, 9H), 1.35-1.16 (m, 12H) ppm. ¹³C NMR: (126 MHz, CDCl₃) δ 159.31, 137.38, 135.34, 133.06, 130.21, 130.10, 128.27, 61.07, 46.70, 45.47, 23.76, 21.61, 20.96 ppm. Specific rotation:

$$[ α ] \frac{23}{D} = - 7.86$$

(c 1.00, CHCl₃) HRMS: Calc'd for C₁₇H₂₈ClN₂O₂S [M+H⁺] 359.1555; found 359.1560. Enantiomeric excess: 97% ee. HPLC Conditions: Daicel Chiralpak IC column, 70:30 n-hexane:i-PrOH, flow rate: 1 mL min-1, 25° C., UV detection wavelength: 220 nm, retention time: major: 12.227 min, minor: 14.347 min.

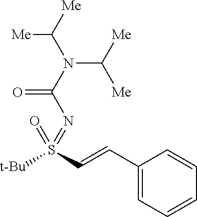 [SMILES: CC(C)N(C(=O)N=[S@](=O)(/C=C/c1ccccc1)C(C)(C)C)C(C)C]

GP-1 was followed with no additional modifications: Commercially available β-bromostyrene (0.375 mmol, 1.5 eq.) was used. Purified by silica gel column chromatography using hexane/EtOAc (0% to 40% EtOAc gradient) to give the product (49.0 mg, 140 μmol, 56% yield) as a white amorphous solid. Only Z isomer was detected after purification.

Physical characteristics: White amorphous solid. TLC: R_{f}=0.20 (hexane/EtOAc, 30% EtOAc). ¹H NMR: (500 MHz, CDCl₃) δ 7.56-7.51 (m, 3H), 7.41-7.37 (m, 3H), 6.92 (d, J=15.5 Hz, 1H), 4.05 (s, 2H), 1.46 (s, 9H), 1.35-1.14 (m, 12H) ppm. ¹³C NMR: (126 MHz, CDCl₃) δ 159.46, 146.18, 133.28, 130.80, 129.02, 128.72, 122.06, 60.49, 45.25, 23.73, 21.55, 20.91 ppm. Specific rotation:

$$[ α ] \frac{23}{D} = + 0.64$$

(c 1.00, CHCl₃) HRMS: Calc'd for C₁₉H₃₁N₂O₂S [M+H⁺] 351.2101; found 351.2105. Enantiomeric excess: 98% ee. HPLC Conditions: Daicel Chiralpak IC column, 70:30 n-hexane:i-PrOH, flow rate: 1 mL min-1, 25° C., UV detection wavelength: 220 nm, retention time: minor: 14.640 min, major: 16.007 min.

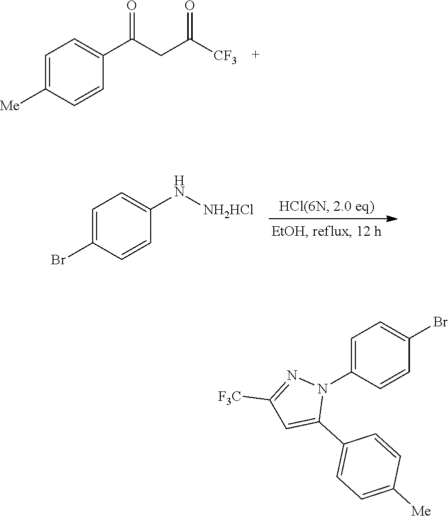

Prepared from a known procedure (J. Am. Chem. Soc. 2020, 142, 38, 16205-16210) to afford a white amorphous solid (3.5 g, 92%).

Physical characteristics: White amorphous solid. TLC: R_{f}=0.35 (hexane/EtOAc, 10% EtOAc). ¹H NMR: (500 MHz, CDCl₃) δ 7.84 (dd, J=6.7, 2.3 Hz, 1H), 7.64 (ddd, J=8.7, 4.3, 2.3 Hz, 1H), 7.29 (t, J=8.5 Hz, 1H), 4.04 (d, J=96.8 Hz, 2H), 1.39 (s, 9H), 1.33 (s, 6H), 1.17 (dd, J=24.9, 6.7 Hz, 6H) ppm. ¹³C NMR: ¹³C NMR (126 MHZ, Chloroform-d) δ 144.99, 143.63 (q, J=38.4 Hz), 139.54, 138.47, 132.37, 129.70, 128.83, 126.99, 126.13, 122.29, 121.34 (q, J=269.0 Hz), 105.82 (d, J=2.2 Hz), 21.45 ppm. 19F NMR: (471 MHZ, CDCl₃) δ −107.84 ppm. HRMS: Calc'd for C₁₇H₁₃BrF₃N₂ [M+H⁺] 381.0209; found 381.0206.

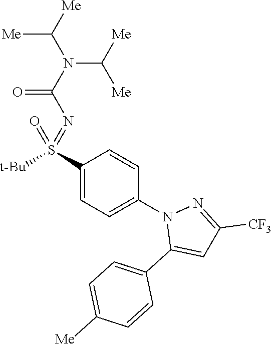 [SMILES: Cc1ccc(-c2cc(C(F)(F)F)nn2-c2ccc([S@@](=O)(=NC(=O)N(C(C)C)C(C)C)C(C)(C)C)cc2)cc1]

GP-1 was followed with no additional modifications: The halide prepared above (0.375 mmol, 1.5 eq.) was used. Purified by silica gel column chromatography using DCM/EtOAc (0% to 10% EtOAc gradient) to give the product (95.9 mg, 175 μmol, 70% yield) as a white solid.

TLC: R_{f}=0.35 (hexane/EtOAc, 30% EtOAc). ¹H NMR: (500 MHz, CDCl₃) δ 7.84 (dd, J=6.7, 2.3 Hz, 1H), 7.64 (ddd, J=8.7, 4.3, 2.3 Hz, 1H), 7.29 (t, J=8.5 Hz, 1H), 4.04 (d, J=96.8 Hz, 2H), 1.39 (s, 9H), 1.33 (s, 6H), 1.17 (dd, J=24.9, 6.7 Hz, 6H) ppm. ¹³C NMR: (126 MHZ, CDCl₃) δ 159.18, 145.36, 144.11 (q, J=38.5 Hz), 142.72, 139.78, 134.79, 131.15, 129.83, 128.83, 125.74, 125.02, 121.16 (q, J=269.2 Hz), 106.51-106.22 (m), 61.00, 47.12, 45.33, 23.61, 21.37 ppm. 19F NMR: (471 MHz, CDCl₃) δ −107.84 ppm. Specific rotation:

$$[ α ] \frac{23}{D} = + 42.52$$

(c 1.00, CHCl₃) HRMS: Calc'd for C₂₈H₃₅F₃N₄NaO₂S [M+Na⁺] 571.2325 found 571.2330. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 7.687 min, minor: 12.373 min.

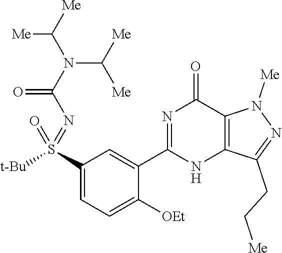 [SMILES: CCCc1nn(C)c2c(=O)nc(-c3cc([S@@](=O)(=NC(=O)N(C(C)C)C(C)C)C(C)(C)C)ccc3OCC)[nH]c12]

GP-3 was followed with additional modifications mentioned: The aryl bromide was prepared using a known procedure.² MeLi (0.124 mL, 0.384 mmol, 3.1 M, 1.2 eq.) was added to a solution of the aryl bromide (0.320 mmol, 1 eq.) in Et₂O (2.5 mL) at −78° C. and stirred for 30 minutes followed by the addition of n-BuLi (0.30 mL, 0.48 mmol, 1.6 M, 1.5 eq.). The reaction mixture stirred at −78° C. for 1 hour before warming to −40° C. where it stirred for an additional 30 minutes before cooling to −78° C. A solution of t-BuSF (128 mg, 0.48 mmol, 1.5 eq.) in Et₂O (0.7 mL) was added dropwise at −78° C. then warmed to −20° C. over 1.5 hours. No modification to the quench and work-up were made. Purification by silica gel column chromatography using Hex/EtOAc (0% to 60% EtOAc gradient) provided the product (96.0 mg, 0.172 mmol, 54% yield) as a white amorphous solid.

Physical characteristic: White amorphous solid. TLC: R_{f}=0.39 (DCM/MeOH, 5% MeOH). ¹H NMR: (500 MHz, CDCl₃) δ 10.93 (s, 1H), 8.80 (d, J=2.4 Hz, 1H), 7.94 (dd, J=8.8, 2.4 Hz, 1H), 7.18 (d, J=8.8 Hz, 1H), 4.37 (qd, J=7.0, 1.3 Hz, 2H), 4.26 (s, 3H), 4.15-4.06 (m, 1H), 4.06-3.93 (m, 1H), 2.87 (t, J=7.5 Hz, 2H), 1.86-1.77 (m, 2H), 1.64 (t, J=7.0 Hz, 3H), 1.42 (s, 9H), 1.40 (d, J=7.6 Hz, 6H), 1.14 (d, J=6.8 Hz, 3H), 1.10 (d, J=6.8 Hz, 3H), 0.98 (t, J=7.4 Hz, 3H) ppm. ¹³C NMR: ¹³C NMR (126 MHZ, CDCl₃) δ 159.5, 159.5, 153.7, 146.8, 146.8, 138.4, 134.3, 132.9, 128.2, 124.5, 120.8, 113.2, 66.1, 60.9, 45.2, 38.2, 29.7, 27.6, 23.6, 22.3, 21.8, 20.8, 20.7, 14.6, 14.0 ppm. Specific rotation:

$$[ α ] \frac{22.5}{D} = - 55.56$$

(c 0.8, CHCl₃) HRMS: Calc'd for C₂₈H₄₂N₆O₄SNa [M+Na⁺] 581.2880 found 581.2886. Enantiomers were unable to be separated.

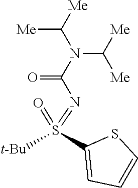 [SMILES: CC(C)N(C(=O)N=[S@](=O)(c1cccs1)C(C)(C)C)C(C)C]

GP-1 was followed with no additional change: Commercially available thiophene (0.375 mmol, 1.5 eq.) was used. Purified by silica gel column chromatography using hexane/EtOAc (0% to 40% EtOAc gradient) to give the product (66.0 mg, 200 μmol, 80% yield) as a white amorphous solid.

Physical characteristics: White amorphous solid. TLC: R_{f}=0.25 (hexane/EtOAc, 30% EtOAc). ¹H NMR: (500 MHz, CDCl₃) δ 7.71 (dd, J=5.0, 1.4 Hz, 1H), 7.55 (dd, J=3.7, 1.4 Hz, 1H), 7.15 (dd, J=5.0, 3.7 Hz, 1H), 4.23-3.84 (m, 2H), 1.45 (s, 9H), 1.30 (d, J=7.2 Hz, 6H), 1.20 (d, 6H) ppm. ¹³C NMR: (126 MHZ, CDCl₃) δ 159.12, 136.07, 135.66, 134.35, 127.95, 61.74, 46.83, 45.31, 23.85, 21.70, 20.94, 20.86 ppm. Specific rotation:

$$[ α ] \frac{23}{D} = - 28.81$$

(c 1.00, CHCl₃) HRMS: Calc'd for C₁₅H₂₇N₂O₂S₂ [M+H⁺] 331.1508; found 331.1509. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 20.673 min, minor: 23.813 min.

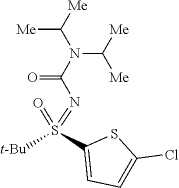 [SMILES: CC(C)N(C(=O)N=[S@](=O)(c1ccc(Cl)s1)C(C)(C)C)C(C)C]

GP-1 was followed with no additional change: Commercially available 2-bromo-5-chloro-thiophene (0.375 mmol, 1.5 eq.) was used. Purified by silica gel column chromatography using hexane/EtOAc (0% to 45% EtOAc gradient) to give the product (74.6 mg, 205 μmol, 82% yield) as a white amorphous solid.

GP-2 was used for a gram scale synthesis with no change in yield or enantiopurity of product.

Physical characteristics: White amorphous solid. TLC: R_{f}=0.50 (hexane/EtOAc, 33% EtOAc). ¹H NMR: (500 MHZ, CDCl₃) δ 7.33 (d, J=4.0 Hz, 1H), 6.99 (d, J=4.0 Hz, 1H), 4.03 (s, 2H), 1.47-1.44 (m, 10H), 1.28-1.20 (m, 12H) ppm. ¹³C NMR: (126 MHz, CDCl₃) δ 158.87, 139.33, 135.05, 134.11, 127.65, 61.99, 46.05, 23.82, 21.30 ppm. Specific rotation:

$$[ α ] \frac{23}{D} = - 33.04$$

(c 1.00, CHCl₃) HRMS: Calc'd for C₁₅H₂₆ClN₂O₂S₂ [M+H⁺] 365.1119; found 365.1122. Enantiomeric excess: >99% ee. HPLC Conditions: Daicel Chiralpak IA column, 95:05 n-hexane:i-PrOH, flow rate: 1 mL min-1, 25° C., UV detection wavelength: 220 nm, retention time: minor: 12.760 min, major: 14.920 min.

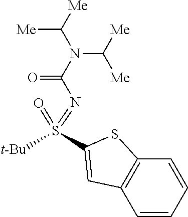 [SMILES: CC(C)N(C(=O)N=[S@](=O)(c1cc2ccccc2s1)C(C)(C)C)C(C)C]

GP-1 was followed with no additional change: Commercially available benzo[b]thiophene (0.375 mmol, 1.5 eq.) was used. Purified by silica gel column chromatography using hexane/EtOAc (0% to 50% EtOAc gradient) to give the product (85.5 mg, 225 μmol, 90% yield) as a white amorphous solid.

Physical characteristics: White amorphous solid. TLC: R_{f}=0.10 (hexane/EtOAc, 30% EtOAc). ¹H NMR: (500 MHz, CDCl₃) δ 7.90-7.81 (m, 3H), 7.48-7.40 (m, 2H), 4.08 (s, 2H), 1.51 (s, 9H), 1.26 (s, 12H) ppm. ¹³C NMR: (126 MHz, CDCl₃) δ 159.14, 143.63, 138.39, 137.06, 133.20, 127.03, 125.81, 125.26, 122.58, 62.08, 45.46, 23.98, 21.79, 21.03 ppm. Specific rotation:

$$[ α ] \frac{23}{D} = - 11.42$$

(c 1.00, CHCl₃) HRMS: Calc'd for C₁₉H₂₉N₂O₂S₂ [M+H⁺] 381.1665; found 381.1668. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 33.574 min, major: 40.653 min.

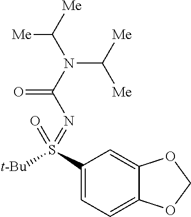 [SMILES: CC(C)N(C(=O)N=[S@](=O)(c1ccc2c(c1)OCO2)C(C)(C)C)C(C)C]

GP-1 was followed with no additional change: Commercially available 5-bromobenzo[d][1,3]dioxole (0.375 mmol, 1.5 eq.) was used. Purified by silica gel column chromatography using hexane/EtOAc (0% to 40% EtOAc gradient) to give the product (68.1 mg, 185 μmol, 74% yield) as a white amorphous solid.

GP-2 was also used to give no significant change in yield or enantiopurity of the product.

Physical characteristics: White amorphous solid. TLC: R_{f}=0.20 (hexane/EtOAc, 50% EtOAc). ¹H NMR: (500 MHz, CDCl₃) δ 7.32 (dd, J=8.2, 1.8 Hz, 1H), 7.17 (s, 1H), 6.91 (d, J=8.2 Hz, 1H), 6.09-6.03 (m, 2H), 4.06 (d, J=35.2 Hz, 2H), 1.37 (s, 9H), 1.36-1.29 (m, 6H), 1.16 (dd, J=21.9, 6.8 Hz, 6H) ppm. ¹³C NMR: (126 MHz, CDCl₃) δ 159.55, 151.85, 148.29, 128.20, 125.82, 110.24, 108.49, 102.40, 61.04, 46.34, 45.29, 23.80, 21.89, 21.83, 20.96, 20.87 ppm. Specific rotation:

$$[ α ] \frac{23}{D} = + 1.57$$

(c 1.00, CHCl₃) HRMS: Calc'd for C₁₈H₂₉N₂O₄S [M+H⁺] 369.1843; found 369.1840. Enantiomeric excess: 98% ee. HPLC Conditions: Daicel Chiralpak IB column, 95:05 n-hexane:i-PrOH, flow rate: 1 mL min-1, 25° C., UV detection wavelength: 220 nm, retention time: major: 19.647 min, minor: 23.007 min.

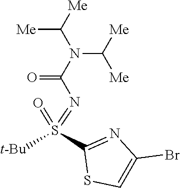 [SMILES: CC(C)N(C(=O)N=[S@](=O)(c1nc(Br)cs1)C(C)(C)C)C(C)C]

GP-1 was followed with no additional change: Commercially available 4-bromothiazole (0.375 mmol, 1.5 eq.) was used. Purified by silica gel column chromatography using hexane/EtOAc (0% to 50% EtOAc gradient) to give the product (72.8 mg, 178 μmol, 71% yield) as a white crystalline solid.

Physical characteristics: White crystalline solid. TLC: R_{f}=0.30 (hexane/EtOAc, 40% EtOAc). ¹H NMR: (500 MHZ, CDCl₃) δ 7.62 (s, 1H), 4.01 (s, 2H), 1.53 (s, 9H), 1.34-1.27 (m, 6H), 1.15 (dd, J=21.5, 6.8 Hz, 6H) ppm. ¹³C NMR: (126 MHZ, CDCl₃) δ 165.67, 158.42, 126.77, 125.50, 62.52, 46.81, 45.63, 23.99, 21.76, 20.76, 20.63 ppm. Specific rotation:

$$[ α ] \frac{23}{D} = - 32.43$$

(c 1.00, CHCl₃) HRMS: Calc'd for C₁₄H₂₄BrN₃NaO₂S₂ [M+Na⁺] 432.0386; found 432.0363. Melting Point: 140-142° C. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 8.553 min, minor: 10.347 min.

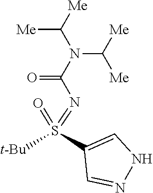 [SMILES: CC(C)N(C(=O)N=[S@](=O)(c1cn[nH]c1)C(C)(C)C)C(C)C]

GP-3 was followed with additional changes mentioned: Commercially available 4-bromopyrazole (0.375 mmol, 1.5 eq.) was used with an increased equivalents of n-BuLi (from 1.5 to 3 eq.). The reaction was warmed to room temperate to be complete. The compound is not UV active and PMA stain was used TLC visualization. Purified by silica gel column chromatography using DCM/MeOH (0% to 10% MeOH gradient) to give the product (62 mg, 197.5 μmol, 79% yield) as a white amorphous solid.

Physical characteristics: White amorphous solid. TLC: R_{f}=0.40 (DCM/MeOH, 10% MeOH) ¹H NMR: (500 MHz, CDCl₃) δ 7.62 (s, 2H), 4.36-3.78 (m, 2H), 1.38 (s, 9H), 1.30 (d, J=7.5 Hz, 12H) ppm. ¹³C NMR: (126 MHZ, CDCl₃) δ 160.55, 137.17, 113.69, 60.53, 47.06, 45.57, 23.17, 21.40, 20.97 ppm. Specific rotation:

$$[ α ] \frac{23}{D} = - 76.09$$

(c 1.00, CHCl₃) HRMS: Calc'd for C₁₄H₂₇N₄O₂S [M+H⁺] 315.1849; found 315.1840.

Enantiomeric excess: 95% ee. HPLC Conditions: Daicel Chiralpak IC column, 70:30 n-hexane; i-PrOH, flow rate: 1 mL min-1, 25° C., UV detection wavelength: 220 nm, retention time: minor: 13.313 min, major: 14.873 min.

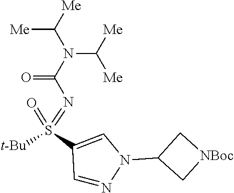 [SMILES: CC(C)N(C(=O)N=[S@](=O)(c1cnn(C2CN(C(=O)OC(C)(C)C)C2)c1)C(C)(C)C)C(C)C]

GP-3 was followed with additional changes mentioned: tert-butyl 3-(4-bromo-1H-pyrazol-1-yl) azetidine-1-carboxylate was prepared by a known procedure.³ CPME was used as the solvent instead of Et₂₀ and 2.2 eq. of t-BuLi was used. The reaction was warmed −20° C. before quenching (full Li—Br exchange was not achieved). Purified by silica gel column chromatography using Hex/EtOAc (0% to 100% EtOAc gradient) to give the product (65 mg, 0.138 mmol, 55% yield) as a colorless foam.

Physical characteristics: Colorless foam. TLC: R_{f}=0.19 (hexanes/EtOAc, 80% EtOAc) ¹H NMR: (500 MHZ, CDCl₃) δ 7.85 (s, 1H), 7.60 (s, 1H), 4.99 (ut, J=7.7, 5.5 Hz, 1H), 4.34-4.26 (m, 4H), 4.06 (s, 1H), 3.83 (s, 1H), 1.39 (s, 9H), 1.34 (s, 9H), 1.19 (d, J=6.9 Hz, 6H), 1.14 (d, J=5.5 Hz, 3H), 1.11 (d, J=5.8 Hz, 3H) ppm. ¹³C NMR: (126 MHZ, CDCl₃) δ 159.2, 156.0, 141.1, 133.5, 116.8, 80.4, 60.4, 56.3, 50.9, 46.6, 45.2, 28.3, 23.3,

$$[ α ] \frac{22.4}{D} = - 63.03$$

(c 1.00, CHCl₃) HRMS: Calc'd 21.5, 21.1, 20.8 ppm. Specific rotation: for C₂₂H₄₀N₅O₄S [M+H⁺] 470.2796; found 470.2787. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 51.29 min, major: 35.48 min.

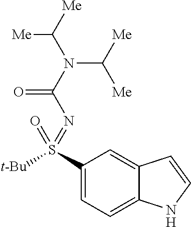 [SMILES: CC(C)N(C(=O)N=[S@](=O)(c1ccc2[nH]ccc2c1)C(C)(C)C)C(C)C]

GP-3 was followed with additional changes mentioned: Commercially available 5-iodo-1H-indole (0.275 mmol, 1.1 eq.) was used. CPME was used instead of Et₂₀ and 2.5 eq. of t-BuLi was used for the Li—I exchange (−78° C., 75 minutes). Warmed to −20° C. and held for 1 hour before quenching. No modifications to the quench or work-up procedure were made. Purified by silica gel column chromatography using Hex/EtOAc (0% to 60% EtOAc gradient) to give the product (58 mg, 0.160 mmol, 64% yield) as a white amorphous solid.

Physical characteristics: White amorphous solid. TLC: R_{f}=0.16 (hexanes/EtOAc, 60% EtOAc) ¹H NMR: ¹H NMR (500 MHZ, CDCl₃) δ 10.31 (s, 1H), 7.84 (d, J=2.0 Hz, 1H), 7.10-7.04 (m, 2H), 6.66 (d, J=8.5 Hz, 1H), 6.28 (s, 1H), 4.49-4.23 (m, 1H), 4.16-3.96 (m, 1H), 1.46-1.38 (m, 6H), 1.35 (s, 9H), 1.34-1.28 (m, 6H) ppm. ¹³C NMR: ¹³C NMR (126 MHz, CDCl₃) δ 160.47, 137.82, 127.54, 127.18, 124.04, 122.46, 120.71, 111.69, 102.28, 61.00, 46.94, 45.23, 23.60, 21.59, 21.06 ppm. Specific rotation:

$$[ α ] \frac{22.2}{D} = - 37.62$$

(c 0.90, CHCl₃) HRMS: Calc'd for C₁₉H₂₉N₃O₂SNa [M+Na⁺] 386.1873; found 386.1871. Enantiomeric excess: >99% ee. HPLC Conditions: Daicel Chiralpak IB column, 90:10 n-hexane:i-PrOH, flow rate: 1 mL/min, 25° C., UV detection wavelength: 254 nm, retention time: minor: 7.94 min, major: 7.09 min.

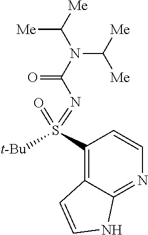 [SMILES: CC(C)N(C(=O)N=[S@](=O)(c1ccnc2[nH]ccc12)C(C)(C)C)C(C)C]

GP-3 was followed with additional changes mentioned: Commercially available 4-bromo-1H-pyrrolo[2,3-b]pyridine (0.275 mmol, 1.1 eq.) was used. CPME was used instead of Et₂₀ and 2.5 eq. of t-BuLi was used for the Li—I exchange (−78° C., 70 minutes: full exchange). Warmed to −10° C. and held for 2 hours before quenching. No modifications to the quench or work-up procedure were made. Purified by silica gel column chromatography using Hex/EtOAc (0% to 60% EtOAc gradient) to give the product (75 mg, 0.208 mmol, 82% yield) as a clear colorless oil.

Physical characteristics: Clear colorless oil. TLC: R_{f}=0.24 (hexanes/EtOAc, 60% EtOAc) ¹H NMR: (500 MHZ, CDCl₃) δ 10.55 (s, 1H), 8.35 (d, J=4.9 Hz, 1H), 7.50-7.29 (m, 1H), 7.24 (t, J=2.9 Hz, 1H), 6.75-6.70 (m, 1H), 4.41-4.16 (m, 1H), 4.08-3.86 (m, 1H), 1.41 (d, J=6.7 Hz, 6H), 1.39 (s, 9H), 1.23 (d, J=6.7 Hz, 3H), 1.16 (d, J=6.6 Hz, 3H) ppm. ¹³C NMR: (126 MHz, CDCl₃) δ 159.7, 149.6, 141.6, 134.5, 128.7, 119.1, 117.1, 101.1, 62.0, 46.7, 45.4, 23.7, 21.9, 21.7, 20.9, 20.8 ppm. Specific rotation:

$$[ α ] \frac{25.6}{D} = - 3.57$$

(c 1.00, CHCl₃) HRMS: Calc'd for C₁₈H₂₈N₄O₂SNa [M+Na⁺] 387.1825; found 387.1829. Enantiomeric excess: 97% ee. HPLC Conditions: Daicel Chiralpak IB column, 95:5 n-hexane:i-PrOH, flow rate: 1 mL/min, 25° C., UV detection wavelength: 220 nm, retention time: minor: 21.31 min, major: 25.84 min.

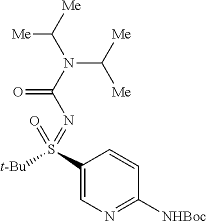 [SMILES: CC(C)N(C(=O)N=[S@](=O)(c1ccc(NC(=O)OC(C)(C)C)nc1)C(C)(C)C)C(C)C]

GP-3 was followed with additional changes mentioned: commercially available tert-butyl (5-bromopyridin-2-yl) carbamate (0.375 mmol, 1.5 eq.) was used. The reaction was warmed to 0° C. to be complete. Purified by silica gel column chromatography using hexane/EtOAc (0% to 40% EtOAc gradient) to give the product (57.2 mg, 130 μmol, 52% yield) as a white amorphous solid.

Physical characteristics: White amorphous solid. TLC: R_{f}=0.25 (hexane/EtOAc, 40% EtOAc). ¹H NMR: (500 MHZ, CDCl₃) δ 8.61 (d, J=2.5 Hz, 1H), 8.52 (s, 1H), 8.13 (d, J=8.9 Hz, 1H), 7.92 (dd, J=9.0, 2.4 Hz, 1H), 4.14 (s, 1H), 3.93 (s, 1H), 1.54 (s, 9H), 1.37 (s, 9H), 1.34-1.29 (m, 6H), 1.16 (dd, J=21.0, 6.8 Hz, 6H) ppm. ¹³C NMR: (126 MHZ, CDCl₃) δ 159.21, 155.40, 152.13, 149.93, 140.13, 125.22, 111.82, 82.24, 61.03, 46.71, 45.35, 28.35, 23.58, 21.77, 21.70, 20.90, 20.83 ppm. Specific rotation:

$$[ α ] \frac{23}{D} = + 41.19$$

(c 0.50, CHCl₃) HRMS: Calc'd for C₂₁H₃₇N₄O₄S [M+H⁺] 441.2530; found 441.2537. Enantiomeric excess: 91% ee. HPLC Conditions: Daicel Chiralpak IB column, 95:05 n-hexane:i-PrOH, flow rate: 1 mL min-1, 25° C., UV detection wavelength: 220 nm, retention time: minor: 14.873 min, major: 18.100 min.

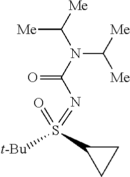 [SMILES: CC(C)N(C(=O)N=[S@](=O)(C1CC1)C(C)(C)C)C(C)C]

GP-2 was followed with additional changes mentioned: commercially available cyclopropylbromide (0.375 mmol, 1.5 eq.) was used and lithiated with t-BuLi (3 eq.) at −78° C. Purified by silica gel column chromatography using hexane/EtOAc (0% to 60% EtOAc gradient) to give the product (51.0 mg, 176 μmol, 70% yield) as a white crystalline solid.

### Different Scale Reactions:

GP-2 was used with t-BuSF (5.65 g, 21.2 mmol, 1 eq.), cyclopropyl bromide (3.40 mL, 42.4 mmol, 2 eq.), and t-BuLi (25 mL, 42.4 mmol, 1.7 M, 2 eq.) to give an identical yield and enantiopurity (4.28 g, 14.8 mmol, 70% yield, 97% ee).

Physical characteristics: White crystalline solid. TLC: R_{f}=0.2 (hexane/EtOAc, 40% EtOAc). ¹H NMR: (500 MHZ, CDCl₃) δ 4.00 (s, 2H), 2.51-2.40 (m, 1H), 1.60 (ddt, J=10.2, 7.5, 5.1 Hz, 1H), 1.48 (s, 9H), 1.29-1.12 (m, 14H), 1.11-1.01 (m, 1H) ppm. 13C NMR: (126 MHz, CDCl₃) δ 158.80, 62.55, 46.48, 45.21, 24.76, 24.37, 21.42, 21.17, 7.48, 5.15 ppm. Specific rotation:

$$[ α ] \frac{23}{D} = + 15.99$$

(c 1.00, CHCl₃) HRMS: Calc'd for C₁₄H₂₉N₂O₂S [M+H⁺] 289.1944; found 289.2952. Melting Point: 175-177° C. Enantiomeric excess: 97% ee. Recrystallized to >99% ee with >90% recovery. HPLC Conditions: Daicel Chiralpak IC column, 70:30 n-hexane:i-PrOH, flow rate: 1 mL min-1, 25° C., UV detection wavelength: 220 nm, retention time: minor: 10.773 min, major: 11.887 min.
Second Functionalization of t-BuSF Reagent: Chiral Sulfonimidoyl Fluoride, Sulfoximine and Sulfonimidamide Product Characterization Data.

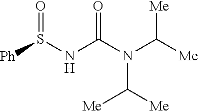

GP-4 was followed with no additional change: tert-butyl phenyl sulfoximine of >99% ee was used (97 mg, 0.30 mmol, 1 eq.). Purified by a short plug of silica gel eluting with DCM to give the product (71.0 mg, 0.264 mmol, 88% yield) as a white amorphous solid.

### Different Scale Reactions:

(1.24 g, 3.82 mmol, >99% ee) produced (0.88 g, 3.28 mmol, 86% yield, >99% ee)
(3.01 g, 9.28 mmol, >99% ee) produced (2.22 g, 8.27 mmol, 89% yield, >99% ee)

Physical characteristics: White amorphous solid. TLC: R_{f}=0.33 (hexane/acetone, 33% acetone). ¹H NMR: (500 MHZ, CDCl₃) δ 7.61 (dd, J=7.9, 1.8 Hz, 2H), 7.48-7.42 (m, 3H), 7.37 (s, 1H), 3.74 (hept, J=6.8 Hz, 2H), 1.24 (d, J=4.1 Hz, 6H), 1.23 (d, J=4.1 Hz, 6H) ppm. ¹³C NMR: (126 MHZ, CDCl₃) δ 153.70, 144.38, 131.27, 129.18, 125.22, 46.88, 21.29, 20.90 ppm. Specific rotation:

$$[ α ] \frac{23}{D} = - 149.55$$

(c 1.00, CHCl₃) HRMS: Calc'd for C₁₃H₂₀N₂O₂SNa [M+Na⁺] 291.1138; found 291.1144. Enantiomeric excess: >99% ee. HPLC Conditions: Daicel Chiralpak IA column, 50:50 n-hexane: DCM, flow rate: 1 mL min-1, 25° C., UV detection wavelength: 220 nm, retention time: minor: 14.55 min, major: 18.83 min.

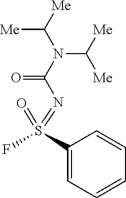 [SMILES: CC(C)N(C(=O)N=[S@@](=O)(F)c1ccccc1)C(C)C]

GP-6 was followed and no additional change: tert-butyl phenyl sulfoximine of >99% ee was used (500 mg, 1.5 mmol, 1.0 eq). Purified by silica gel column chromatography using hexane/EtOAc (0% to 25% EtOAc gradient) to give the product (370 mg, 1.3 mmol, 84% yield) as a white crystalline solid.

### Different Scale Reactions:

(1.0 g, 3.08 mmol, >99% ee) produced (689 mg, 2.41 mmol, 78% yield, >99% ee)
(2.0 g, 6.16 mmol, >99% ee) produced (1.31 g, 4.57 mmol, 74% yield, >99% ee)

Physical characteristics: White crystalline solid. TLC: R_{f}=0.3 (hexane/EtOAc, 20% EtOAc). ¹H NMR: (500 MHz, CDCl₃) δ 8.09-8.02 (m, 2H), 7.76-7.69 (m, 1H), 7.61 (t, J=7.9 Hz, 2H), 4.16 (s, 1H), 3.84 (s, 1H), 1.31 (dd, J=6.9, 3.4 Hz, 6H), 1.21 (t, J=6.6 Hz, 6H) ppm. ¹³C NMR: (126 MHz, CDCl₃) δ 153.30 (d, J=3.2 Hz), 135.39 (d, J=22.6 Hz), 134.98, 129.56, 127.72, 48.28, 45.89, 21.25, 20.59, 20.52 ppm. 19F NMR: (471 MHZ, CDCl₃) δ 69.53 ppm. Specific rotation:

$$[ α ] \frac{23}{D} = + 3 0.26$$

(c 1.00, CHCl₃) HRMS: Calc'd for C₁₃H₂₀FN₂O₂S [M+H⁺] 287.1224; found 287.1224. Melting Point: 102-105° C. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 13.293 min, major: 15.493 min.

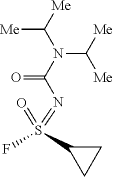 [SMILES: CC(C)N(C(=O)N=[S@@](=O)(F)C1CC1)C(C)C]

GP-6 was followed and no additional change: tert-butyl cyclopropyl sulfoximine of >99% ee was used (1.04 mmol, 1.0 eq) was used. Purified by silica gel column chromatography using hexane/EtOAc (0% to 25% EtOAc gradient) to give the product (232 mg, 0.927 mmol, 89% yield) as colorless oil which slowly solidified into a white amorphous solid.

Physical characteristics: White amorphous solid. TLC: R_{f}=0.3 (hexane/EtOAc, 20% EtOAc). ¹H NMR: (500 MHZ, CDCl₃) δ 4.15 (s, 1H), 3.80 (s, 1H), 3.36 (dq, J=8.2, 3.9 Hz, 1H), 1.55-1.43 (m, 2H), 1.33-1.27 (m, 6H), 1.25 (dq, J=7.9, 1.7 Hz, 2H), 1.19 (d, J=6.4 Hz, 6H) ppm. ¹³C NMR: (126 MHZ, CDCl₃) δ 154.09 (d, J=3.3 Hz), 48.11, 45.72, 30.10 (d, J=27.0 Hz), 20.90 (d, J=72.0 Hz), 7.24, 6.50 ppm. 19F NMR: (471 MHZ, CDCl₃) § 62.06 ppm. Specific rotation:

$$[ α ] \frac{23}{D} = + 38.58$$

(c 1.00, CHCl₃) HRMS: Calc'd for C₁₀H₂₀N₂O₂S [M+H⁺] 251.1224; found 251.1218. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 7.413 min, major: 8.187 min.

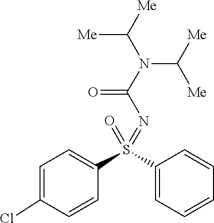

GP-7 (Grignard reagent) was followed with no additional modification: Commercially available 4-chlorophenylmagnesium bromide (0.275 mmol, 1.1 eq., 1.0 M solution in Et₂O) from Sigma-Aldrich was used with Ph-sulfonimidoyl fluoride (0.25 mmol, 1 eq.). Purified by silica gel column chromatography using hexane/EtOAc (0% to 50% EtOAc gradient) to give the product (89.8 mg, 0.237 mmol, 95% yield, >99% ee) as a white amorphous solid.

GP-8 (turbo-Grignard reagent) was followed with additional modifications mentioned: Commercially available 4-chloro-bromobenzene (0.275 mmol, 1.1 eq.) in dry THF (0.2 mL) was added at room temperature in one portion to the stirring isopropylmagnesium chloride lithium chloride complex solution (0.21 mL, 0.275 mmol, 1.1 eq. 1.3 M in THF), exchange takes about 12 hours. Purified by silica gel column chromatography using hexane/EtOAc (0% to 50% EtOAc gradient) to give the product (56.7 mg, 0.15 mmol, 60% yield, >99% ee) as a white amorphous solid.

GP-1 (organolithium) was followed with no additional modifications: Purified by silica gel column chromatography using hexane/EtOAc (0% to 50% EtOAc gradient) to give the product (75.6 mg, 0.2 mmol, 80% yield, 94% ee) as a white amorphous solid.

Physical characteristics: White amorphous solid. TLC: R_{f}=0.6 (hexane/EtOAc, 50% EtOAc). ¹H NMR (600 MHZ, CDCl₃) δ 7.96-7.93 (m, 2H), 7.88-7.84 (m, 2H), 7.56-7.52 (m, 1H), 7.52-7.48 (m, 2H), 7.46-7.42 (m, 2H), 4.12 (br, 2H), 1.28 (s, 12H) ppm. ¹³C NMR (151 MHZ, CDCl₃) δ 158.66, 140.97, 140.23, 139.28, 132.96, 129.74, 129.55, 129.10, 127.68, 46.28, 21.28 ppm. Specific rotation:

$$[ α ] \frac{23}{D} = - 3 \text{.42}$$

(c 1.00, CHCl₃) Melting Point: 129-131° C. HRMS: Calc'd for C₁₉H₂₄ClN₂O₂S⁺ [M+H⁺] 379.1242; found 379.1242. Enantiomeric excess: >99% ee for both Grignard and turbo-Grignard reagents. 94% ee for organolithium reagent. HPLC Conditions: Daicel Chiralpak IC column, 70:30 n-hexane:i-PrOH, flow rate: 1 mL min-1, 25° C., UV detection wavelength: 220 nm, retention time: minor: 18.860 min, major: 22.380 min.

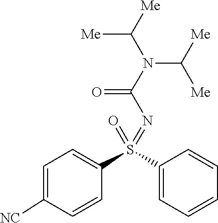

GP-8 was used with additional modifications mentioned: A solution of commercially available 4-iodobenzonitrile (63 mg, 0.275 mmol, 1.1 eq.) in dry THF (0.25 ml) was added to isopropylmagnesium chloride lithium chloride complex solution (0.21 mL, 0.275 mmol, 1.1 eq. 1.3 M in THF) dropwise at −78° C. Upon gradual warming up to −10° C. for 4 hours (the reaction mixture turned yellow), then a solution of Ph-sulfonimidoyl fluoride (0.25 mmol, 1 eq.) in THF (0.25 mL) was added, workup procedure as described in GP-8. Purified by silica gel column chromatography using hexane/EtOAc (0% to 50% EtOAc gradient) to give the product (90.4 mg, 0.245 mmol, 98% yield) as a colorless oil.

GP-12 was followed with the Mg—X exchange as described above with 2 equivalents of turbo-Grignard reagent: Ph-sulfonimidoyl fluoride (0.25 mmol, 1 eq.) was used to provide (65 mg, 176 mmol, 70% yield) as colorless oil.

Physical characteristics: Colorless oil. TLC: R_{f}=0.71 (hexane/EtOAc, 50% EtOAc). 1H NMR (500 MHZ, CDCl₃) δ 8.03-7.99 (m, 2H), 7.99-7.95 (m, 2H), 7.73 (d, J=8.4 Hz, 2H), 7.59-7.54 (m, 1H), 7.51 (dd, J=8.4, 6.7 Hz, 2H), 4.28 (br, 1H), 3.92 (br, 1H), 1.55-0.96 (m, 12H) ppm. ¹³C NMR (126 MHZ, CDCl₃) δ 158.33, 158.33, 146.62, 146.62, 139.48, 139.48, 133.50, 133.50, 133.06, 133.06, 129.69, 129.69, 128.15, 128.15, 127.88, 127.88, 117.41, 117.41, 116.05, 116.05, 47.02, 47.02, 45.60, 45.60, 21.28, 21.28 ppm. Specific rotation:

$$[ α ] \frac{23}{D} = - 19. 16$$

(c 1.00, CHCl₃) HRMS: Calc'd for C₂₀H₂₄N₃O₂S⁺ [M+H⁺] 370.1584; found 370.1576. Enantiomeric excess: >99% ee. HPLC Conditions: Daicel Chiralpak IC column, 70:30 n-hexane:i-PrOH, flow rate: 1 mL min-1, 25° C., UV detection wavelength: 254 nm, retention time: minor: 28.347 min, major: 35.253 min.

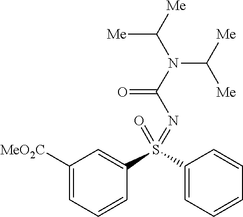

GP-8 was used with additional modifications mentioned: A solution of commercially available methyl 3-iodobenzoate (72.1 mg, 0.275 mmol, 1.1 eq.) in dry THF (0.25 mL) was added to isopropylmagnesium chloride lithium chloride complex solution (0.21 mL, 0.275 mmol, 1.1 eq. 1.3 M in THF) dropwise at −78° C., to. Upon gradual warming up to −10° C. for 4 hours (the reaction mixture turned yellow) then a solution of Ph-sulfonimidoyl fluoride (0.25 mmol, 1 eq.) in THF (0.25 mL) was added. Work-up procedure as described in GP-8. Purified by silica gel column chromatography using DCM/acetone, (0% to 10% Acetone gradient) to give the product (63.4 mg, 0.158 mmol, 63% yield) as a colorless oil.

Physical characteristics: Colorless oil. TLC: R_{f}=0.84 (DCM/acetone, 10% acetone). 1H NMR (500 MHz, CDCl₃) δ 8.58 (t, J=1.8 Hz, 1H), 8.15 (dt, J=7.8, 1.4 Hz, 1H), 8.11 (ddd, J=8.0, 2.0, 1.2 Hz, 1H), 8.01-7.93 (m, 2H), 7.56 (t, J=7.9 Hz, 1H), 7.53-7.46 (m, 3H), 4.19 (br, 2H), 3.90 (s, 3H), 1.29 (s, 12H) ppm. ¹³C NMR (126 MHZ, CDCl₃) δ 165.44, 158.58, 142.39, 140.64, 133.41, 132.94, 131.64, 131.53, 129.64, 129.48, 128.77, 127.68, 52.55, 46.06, 21.15 ppm. Specific rotation:

$$[ α ] \frac{23}{D} = - 8 \text{.82}$$

(c 1.00, CHCl₃) HRMS: Calc'd for C₂₁H₂₇N₂O₄S⁺ [M+H⁺] 403.1686; found 403.1675. Enantiomeric excess: >99% ee. HPLC Conditions: Daicel Chiralpak IB column, 95:05 n-hexane:i-PrOH, flow rate: 1 mL min-1, 25° C., UV detection wavelength: 220 nm, retention time: major: 26.667 min, minor: 29.413 min.

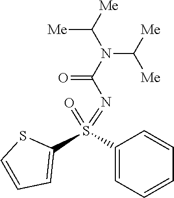 [SMILES: CC(C)N(C(=O)N=[S@@](=O)(c1ccccc1)c1cccs1)C(C)C]

GP-8 was followed with additional modifications mentioned: Commercially available 2-bromothiophene (0.25 mmol, 1 eq.) was added at room temperature in one portion to a stirring isopropylmagnesium chloride lithium chloride complex solution (0.21 mL, 0.275 mmol, 1.1 eq. 1.3 M in THF) (exchange takes about 12 hours) then cooled to 0° C. A solution of Ph-sulfonimidoyl fluoride (0.25 mmol, 1 eq.) in THF (0.25 mL) was added dropwise at 0° C. then warmed to room temperature. Work-up procedure as described in GP-8. Purified by silica gel column chromatography using hexane/EtOAc (0% to 50% EtOAc gradient) to give the product (81.4 mg, 0.233 mmol, 93% yield) as a light-pink crystalline solid.

Physical characteristics: Light-pink crystalline solid TLC: R_{f}=0.46 (hexane/EtOAc, 50% EtOAc). ¹H NMR (500 MHZ, CDCl₃) δ 7.99-7.95 (m, 2H), 7.59 (d, J=4.4 Hz, 2H), 7.53-7.46 (m, 3H), 7.04 (t, J=4.4 Hz, 1H), 4.09 (br, 2H), 1.29 (s, 12H) ppm. ¹³C NMR: (126 MHz, CDCl₃) δ 158.38, 142.44, 142.11, 133.67, 133.13, 132.60, 129.37, 128.23, 127.28,

$$[ α ] \frac{23}{D} = 61.32$$

(c 1.00, CHCl₃) Melting Point: 177-45.75, 21.14 ppm. Specific rotation: 179° C. HRMS: Calc'd for C₁₇H₂₃N₂O₂S₂⁺ [M+H⁺] 351.1195; found 351.1189. Enantiomeric excess: >99% ee. HPLC Conditions: Daicel Chiralpak IC column, 70:30 n-hexane:i-PrOH, flow rate: 1 mL min-1, 25° C., UV detection wavelength: 254 nm, retention time: minor: 25.780 min, major: 29.880 min.

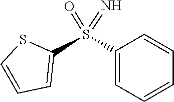 [SMILES: N=[S@@](=O)(c1ccccc1)c1cccs1]

GP-13 was followed with no additional modification: N,N-diisopropyl urea protected thiophene phenyl sulfoximine (52.5 mg, 0.15 mmol, 1 eq.) was used. Purified by silica gel column chromatography using DCM/acetone (0% to 10% acetone) to give the product (25 mg, 112 mmol, 75% yield) as a white crystalline solid.

Physical characteristics: White crystalline solid. TLC: R_{f}=0.7 (DCM/acetone, 10% acetone). ¹H NMR: (500 MHZ, CDCl₃) δ 8.11 (dd, J=7.5, 1.8 Hz, 2H), 7.65 (dd, J=3.8, 1.4 Hz, 1H), 7.59 (dd, J=4.9, 1.4 Hz, 1H), 7.57-7.52 (m, 1H), 7.49 (dd, J=8.4, 6.6 Hz, 2H), 7.04 (dd, J=5.0, 3.8 Hz, 1H), 3.34 (s, 1H) ppm. ¹³C NMR: (126 MHZ, CDCl₃) δ 146.14, 143.33, 133.78, 133.21, 132.72, 129.18, 127.95, 127.65. ppm. Specific rotation:

$$[ α ] \begin{matrix}23 \\ D\end{matrix} = 14.2$$

(c 1.00, CHCl₃) Melting Point: 123-125° C. HRMS: Calc'd for C₁₀H₁₀NOS₂⁺ [M+H⁺] 224.0198; found 224.0198. Enantiomeric excess: >99% ee. HPLC Conditions: Daicel Chiralpak IB column, 90:10 n-hexane:i-PrOH, flow rate: 1 mL min-1, 25° C., UV detection wavelength: 254 nm, retention time: minor: 28.53 min, major: 29.94 min.

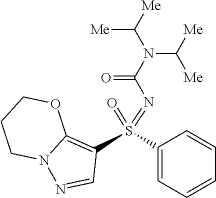 [SMILES: CC(C)N(C(=O)N=[S@@](=O)(c1ccccc1)c1cnn2c1OCCC2)C(C)C]

GP-8 was followed with additional modifications mentioned: 3-iodo-6,7-dihydro-5H-pyrazolo[5,1-b][1,3]oxazine (62.5 mg, 0.25 mmol) in 0.5 mL dry THF was added at 0° C. dropwise to the stirring isopropylmagnesium chloride lithium chloride complex solution (0.21 mL, 0.275 mmol, 1.1 eq. 1.3 M in THF) which was slowly warmed to room temperature (exchange takes about 6 hours, turning into a white emulsion). Then a solution of Ph-sulfonimidoyl fluoride (0.25 mmol, 1 eq.) in THF (0.25 mL) was added. Purified by silica gel column chromatography using DCM/acetone (0% to 15% acetone gradient) to give the product (87.7 mg, 0.225 mmol, 90% yield) as a white crystalline solid.

Physical characteristics: White crystalline solid. TLC: R_{f}=0.46 (hexane/EtOAc, 50% EtOAc). ¹H NMR: (500 MHz, CDCl₃) δ 7.94-7.90 (m, 2H), 7.64 (s, 1H), 7.49-7.41 (m, 3H), 4.29 (dddd, J=36.7, 11.1, 6.6, 4.0 Hz, 2H, overlay with br, 1H), 4.05 (t, J=6.2 Hz, 2H), 3.85 (br, 1H), 2.25-2.13 (m, 2H), 1.28 (d, J=6.9 Hz, 6H), 1.17 (t, J=8.2 Hz, 6H) ppm. ¹³C NMR: (126 MHz, CDCl₃) δ 158.71, 149.68, 142.88, 138.97, 132.13, 128.98, 126.78, 102.22, 66.59, 46.93, 44.98, 44.35, 21.40, 21.35, 21.03, 20.80, 20.74 ppm. Specific rotation:

$$[ α ] \frac{23}{D} = 4 \text{.55}$$

(c 1.00, CHCl₃) Melting Point: 124-125° C. HRMS: Calc'd for C₁₉H₂₇N₄O₃S⁺ [M+H⁺] 391.1798; found 391.1793. Enantiomeric excess: >99% ee. HPLC Conditions: Daicel Chiralpak IB column, 90:10 n-hexane:i-PrOH, flow rate: 1 mL min-1, 25° C., UV detection wavelength: 220 nm, retention time: minor: 34.340 min, major: 42.567 min.

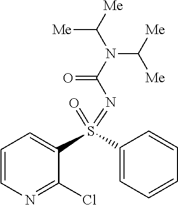 [SMILES: CC(C)N(C(=O)N=[S@@](=O)(c1ccccc1)c1cccnc1Cl)C(C)C]

GP-8 was followed and additional changes mentioned: A solution of isopropylmagnesium chloride lithium chloride complex solution (0.21 mL, 0.275 mmol, 1.1 eq. 1.3 M in THF) was added dropwise at −40° C., to a solution of commercially available 3-iodo-2-chloropyridine (52.9 mg, 0.275 mmol, 1.1 eq.) in 0.25 mL dry THF. Upon gradual warming up to 0° C. over 1 hour, the reaction mixture turned yellow, then a solution of Ph-sulfonimidoyl fluoride (0.25 mmol, 1 eq.) in THF (0.25 mL) was added, workup procedure as described in GP-8. Purified by silica gel column chromatography using DCM/acetone (0% to 9% acetone gradient) to give the product (70.2 mg, 0.185 mmol, 74% yield) as a light-yellow amorphous solid.

Physical characteristics: Light-yellow amorphous solid. TLC: R_{f}=0.73 (DCM/acetone, 10% acetone). ¹H NMR: (500 MHZ, CDCl₃) δ 8.77 (dd, J=7.9, 1.9 Hz, 1H), 8.46 (dd, J=4.7, 1.9 Hz, 1H), 8.08-7.98 (m, 2H), 7.60 (t, J=7.4 Hz, 1H), 7.55-7.45 (m, 3H), 4.38 (br, 1H), 3.84 (br, 1H), 1.34 (d, J=7.0 Hz, 6H), 1.20 (dd, J=21.1, 6.9 Hz, 6H) ppm. ¹³C NMR: (126 MHz, CDCl₃) δ 157.86, 152.51, 146.97, 141.15, 137.49, 137.27, 133.69, 129.13, 129.09, 123.48, 47.39, 45.37, 21.53, 21.44, 20.66, 20.54 ppm. Specific rotation:

$$[ α ] \frac{23}{D} = - 60.07$$

(c 1.00, CHCl₃) Melting Point: 96-97° C. HRMS: Calc'd for C₁₈H₂₃ClN₃O₂S⁺ [M+H⁺] 380.1194; found 380.1188. Enantiomeric excess: 98.7% ee. HPLC Conditions: Daicel Chiralpak IC column, 70:30 n-hexane:i-PrOH, flow rate: 1 mL min-1, 25° C., UV detection wavelength: 254 nm, retention time: minor: 15.013 min, major: 18.127 min.

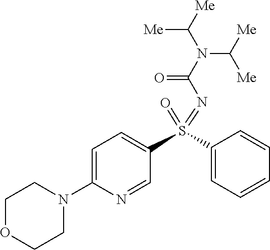 [SMILES: CC(C)N(C(=O)N=[S@@](=O)(c1ccccc1)c1ccc(N2CCOCC2)nc1)C(C)C]

GP-8 was followed with additional changes mentioned: 4-(5-bromopyridin-2-yl) morpholine (60.5 mg, 0.25 mmol, 1 eq.) in 0.3 mL dry THF was added at room temperature in one portion to the stirring isopropylmagnesium chloride lithium chloride complex solution (0.38 mL, 0.5 mmol, 2 eq. 1.3 M in THF) at 0° C. then warmed to room temperature where it stirred for 12 hours (reaction mixture turned turbid orange). Upon complete Mg-halogen exchange, a solution of Ph-sulfonimidoyl fluoride (107.3 mg, 0.375 mmol, 1.5 eq.) in THF (0.5 mL) was added at 0° C. then warmed to room temperature while monitoring by TLC. Work-up conditions were the same as described in GP-8. Purified by silica gel column chromatography using DCM/acetone (0% to 9% acetone gradient) to give the product (64.5 mg, 0.15 mmol, 60% yield) as a colorless crystalline solid.

Physical characteristics: Colorless crystalline solid. TLC: R_{f}=0.2 (hexane/EtOAc, 50% EtOAc, light purple spot under 254 nm UV). ¹H NMR: (500 MHZ, CDCl₃) δ 8.66 (d, J=2.5 Hz, 1H), 7.91 (dd, J=8.0, 1.8 Hz, 2H), 7.88 (dd, J=9.1, 2.6 Hz, 1H), 7.51-7.43 (m, 3H), 6.58 (d, J=9.2 Hz, 1H), 4.29 (s, 1H), 3.94 (s, 1H), 3.76-3.73 (m, 4H), 3.60 (dd, J=5.8, 4.1 Hz, 4H), 1.35 (d, J=6.9 Hz, 6H), 1.19 (t, J=7.9 Hz, 6H) ppm. ¹³C NMR: (126 MHz, CDCl₃) δ 160.11, 158.70, 148.67, 142.26, 136.78, 132.29, 129.31, 127.09, 124.90, 105.77, 66.50, 46.91, 45.23, 44.86, 21.64, 20.75 ppm. Specific rotation:

$$[ α ] \frac{23}{D} = 21.08$$

(c1.00, CHCl₃) Melting Point: 118-120° C. HRMS: Calc'd for C₂₂H₃₁N₄O₃S⁺ [M+H⁺] 431.2111; found 431.2101. Enantiomeric excess: >99% ee. HPLC Conditions: Daicel

Chiralpak IB column, 90:10 n-hexane:i-PrOH, flow rate: 1 mL min-1, 25° C., UV detection wavelength: 220 nm, retention time: minor: 34.567 min, major: 38.660 min.

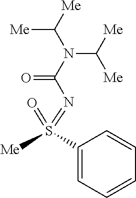 [SMILES: CC(C)N(C(=O)N=[S@@](C)(=O)c1ccccc1)C(C)C]

GP-7 was followed with no additional change: Ph-sulfonimidoyl fluoride (0.25 mmol, 1 eq.) and commercially available methylmagnesium bromide from Sigma-Aldrich were used. Purified by silica gel column chromatography using hexane/EtOAc (0% to 50% EtOAc gradient) to give the product (64.2 mg, 0.227 mmol, 91% yield) as a white crystalline solid.

GP-12 was followed with no additional change: Ph-sulfonimidoyl fluoride (0.25 mmol, 1 eq.) and commercially available methylmagnesium chloride (0.50 mmol, 2 eq.)

from Sigma-Aldrich were used to provide (52 mg, 184 mmol, 74% yield) as white crystalline solid.

Physical characteristics: White crystalline solid. TLC: R_{f}=0.3 (hexane/EtOAc, 33% EtOAc). ¹H NMR: (500 MHz, CDCl₃) δ 7.96-7.91 (m, 2H), 7.63-7.59 (m, 1H), 7.57-7.52 (m, 2H), 4.15 (s, 1H), 3.91 (s, 1H), 3.30 (s, 3H), 1.29-1.16 (m, 12H) ppm. ¹³C NMR: (126 MHz, CDCl₃) δ 159.09, 140.42, 133.12, 129.43, 127.20, 46.79, 45.12, 44.97, 21.47, 21.38, 20.79 ppm. Specific rotation:

$$[ α ] \frac{23}{D} = - 14 \text{.51}$$

(c 1.00, CHCl₃) Melting Point: 169-170° C. HRMS: Calc'd for C₁₄H₂₃N₂O₂S⁺ [M+H⁺] 283.1475; found 283.1474. Enantiomeric excess: >99% ee. HPLC Conditions: Daicel Chiralpak IC column, 70:30 n-hexane: i-PrOH, flow rate: 1 mL min-1, 25° C., UV detection wavelength: 220 nm, retention time: major: 18.687 min, minor: 29.860 min.

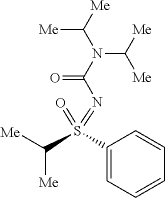 [SMILES: CC(C)N(C(=O)N=[S@@](=O)(c1ccccc1)C(C)C)C(C)C]

GP-7 was followed with no additional change: Ph-sulfonimidoyl fluoride (0.25 mmol, 1 eq.) and commercially available isopropylmagnesium chloride from Sigma-Aldrich were used. Purified by silica gel column chromatography using hexane/EtOAc (0% to 50% EtOAc gradient) to give the product (69.8 mg, 0.225 mmol, 90% yield) as a white amorphous solid.

GP-8 was used with no additional change: Ph-sulfonimidoyl fluoride (0.25 mmol, 1 eq.) and commercially available isopropylmagnesium chloride lithium chloride complex solution (1.3 M in THF) from Sigma-Aldrich. Purified by silica gel column chromatography using hexane/EtOAc (0% to 50% EtOAc gradient) to give the product (73.6 mg, 0.237 mmol, 95% yield) as a white amorphous solid.

GP-12 was followed with no additional change: Ph-sulfonimidoyl fluoride (0.25 mmol, 1 eq.) and commercially available isopropylmagnesium chloride lithium chloride complex solution (0.385 mL, 2 eq., 1.3 M in THF) from Sigma-Aldrich were used to provide (53 mg, 171 mmol, 68% yield) as white amorphous solid.

Physical characteristics: White amorphous solid. TLC: R_{f}=0.44 (hexane/EtOAc, 50% EtOAc). ¹H NMR: (500 MHZ, CDCl₃) δ 7.83-7.80 (m, 2H), 7.61-7.57 (m, 1H), 7.53 (dd, J=8.5, 6.7 Hz, 2H), 4.05 (d, J=70.6 Hz, 2H), 3.58 (pd, J=6.9, 1.0 Hz, 1H), 1.36-1.13 (m, 18H) ppm. ¹³C NMR: (126 MHz, CDCl₃) δ 159.23, 136.23, 132.93, 129.07, 128.95, 56.10, 46.51, 45.16, 21.58, 20.79, 15.99, 15.87 ppm. Specific rotation:

$$[ α ] \frac{23}{D} = 6.00$$

(c 1.00, CHCl₃) Melting Point: 99-110° C. HRMS: Calc'd for C₁₆H₂₇N₂O₂S⁺ [M+H⁺] 311.1788; found 311.1779. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 20.207 min, major: 23.347 min.

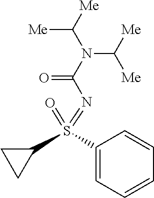 [SMILES: CC(C)N(C(=O)N=[S@@](=O)(c1ccccc1)C1CC1)C(C)C]

GP-7 was followed with no additional change: Ph-sulfonimidoyl fluoride (0.25 mmol, 1 eq.) and commercially available cyclopropylmagnesium chloride from Sigma-Aldrich were used. Purified by silica gel column chromatography using hexane/EtOAc (0% to 50% EtOAc gradient) to give the product (75.4 mg, 0.245 mmol, 98% yield) as a white amorphous solid.

Physical characteristics: White amorphous solid. TLC: R_{f}=0.4 (hexane/EtOAc, 50% EtOAc). ¹H NMR: (500 MHZ, CDCl₃) δ 7.82 (dd, J=7.2, 1.9 Hz, 2H), 7.57-7.53 (m, 1H), 7.50 (dd, J=8.3, 6.4 Hz, 2H), 4.07 (br, 1H), 3.92 (br, 1H), 2.51 (tt, J=8.0, 4.8 Hz, 1H), 1.47 (ddt, J=10.2, 7.3, 5.0 Hz, 1H), 1.32-1.20 (m, 7H), 1.18-1.07 (m, 7H), 0.90 (dtd, J=9.1, 7.5, 5.2 Hz, 1H) ppm. ¹³C NMR: (126 MHz, CDCl₃) δ 158.73, 140.89, 132.61, 129.30, 127.24, 46.53, 45.09, 33.86, 21.59, 20.71, 6.70, 5.36 ppm. Specific rotation:

$$[ α ] \frac{23}{D} = 21 \text{.43}$$

(c 1.00, CHCl₃) Melting Point: 80-81° C. HRMS: Calc'd for C₁₆H₂₅N₂O₂S⁺ [M+H⁺] 309.1632; found 309.1632. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 18.653 min, major: 22.207 min.

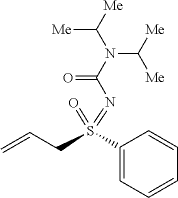 [SMILES: C=CC[S@@](=O)(=NC(=O)N(C(C)C)C(C)C)c1ccccc1]

GP-7 was followed with additional changed mentioned: Allylmagnesium bromide prepared following literature procedure (J. Org. Chem. 1942, 07, 4, 326) and added dropwise to a solution of Ph-sulfonimidoyl fluoride (0.25 mmol, 1 eq.) at −78° C. over 5 minutes to give a gold solution, reaction completed after 1 h at −78° C. and quenched with MeOH. Purified by silica gel column chromatography using hexane/EtOAc (0% to 50% EtOAc gradient) to give the product (76 mg, 0.247 mmol, 98% yield) as a white crystalline solid.

Physical characteristics: White crystalline solid. TLC: R_{f}=0.53 (hexane/EtOAc, 50% EtOAc). ¹H NMR: (500 MHZ, CDCl₃) δ 7.86 (dd, J=7.4, 1.7 Hz, 2H), 7.61-7.56 (m, 1H), 7.51 (dd, J=8.5, 7.0 Hz, 2H), 5.64 (ddt, J=17.5, 10.2, 7.5 Hz, 1H), 5.23 (d, J=10.1 Hz, 1H), 5.03 (dd, J=17.1, 1.6 Hz, 1H), 4.40 (dd, J=13.6, 7.5 Hz, 1H), 4.18 (s, 1H), 4.10 (dd, J=13.6, 7.4 Hz, 1H), 3.89 (s, 1H), 1.23 (d, J=6.8 Hz, 12H) ppm. ¹³C NMR: (126 MHZ, CDCl₃) δ 159.09, 137.39, 133.25, 129.06, 129.02, 128.45, 125.16, 124.63, 60.37, 46.98, 45.13, 21.38, 20.78 ppm. Specific rotation:

$$[ α ] \frac{23}{D} = - 99.33$$

(c 1.00, CHCl₃) Melting Point: 79-80° C. HRMS: Calc'd for C₁₆H₂₅N₂O₂S⁺ [M+H⁺] 309.1632; found 309.1632. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 16.560 min, minor: 21.653 min.

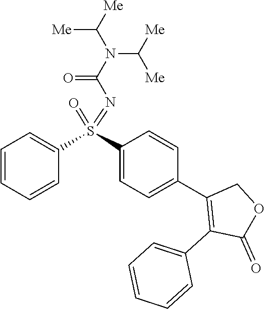 [SMILES: CC(C)N(C(=O)N=[S@@](=O)(c1ccccc1)c1ccc(C2=C(c3ccccc3)C(=O)OC2)cc1)C(C)C]

GP-8 was used with additional changes mentioned: 4-(4-iodophenyl)-3-phenylfuran-2 (5H)-one (60.5 mg, 0.25 mmol, 1 eq.) in 0.3 mL dry THF was added at room temperature in one portion to the stirring isopropylmagnesium chloride lithium chloride complex solution (0.38 mL, 0.5 mmol, 2 eq. 1.3 M in THF), the reaction mixture turned turbid orange and exchange takes about 2 hours. Upon complete Mg-halogen exchange, a solution of Ph-sulfonimidoyl fluoride (107.3 mg, 0.375 mmol, 1.5 eq.) in THF (0.5 mL) was added, workup procedure as described in Method 2. Purified by silica gel column chromatography using DCM/acetone (0% to 6% acetone gradient) to give the product (77.8 mg, 0.155 mmol, 62% yield) as a colorless crystalline solid.

Physical characteristics: Colorless crystalline solid. TLC: R_{f}=0.72 (DCM/acetone, 10% acetone). ¹H NMR: (500 MHz, CDCl₃) δ 7.98-7.93 (m, 2H), 7.90-7.86 (m, 2H), 7.57-7.53 (m, 1H), 7.50 (dd, J=8.3, 6.6 Hz, 2H), 7.44-7.40 (m, 2H), 7.36 (s, 5H), 5.13 (d, J=3.5 Hz, 2H), 4.30 (br, 1H), 3.90 (br, 1H), 1.36-1.32 (m, 6H), 1.19 (t, J=7.4 Hz, 6H) ppm. ¹³C NMR: (126 MHz, CDCl₃) δ 172.80, 158.51, 153.64, 143.53, 140.38, 134.77, 133.05, 129.50, 129.45, 129.31, 129.21, 128.97, 128.93, 128.46, 128.38, 128.17, 127.75, 70.37, 47.15, 45.38, 21.61, 20.70 ppm. Specific rotation:

$$[ α ] \frac{23}{D} = 8.57$$

(c 1.00, CHCl₃) Melting Point: 192-193° C. HRMS: Calc'd for C₂₉H₃₁N₂O₄S⁺ [M+H⁺] 503.2000; found 503.2016. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 19.693 min, major: 22.627 min.

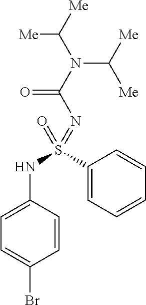 [SMILES: CC(C)N(C(=O)N=[S@](=O)(Nc1ccc(Br)cc1)c1ccccc1)C(C)C]

GP-9 was followed with no additional change: Commercially available 4-bromoaniline (1.0 eq, 0.25 mmol) was used. NaHMDS (2.0 eq) was used as base. Purified by silica gel column chromatography using hexane/EtOAc (0% to 35% EtOAc gradient) to give the product (89 mg, 203 μmol, 81% yield) as a white solid.

Physical characteristics: White amorphous solid. TLC: R_{f}=0.6 (hexane/EtOAc, 50% EtOAc). ¹H NMR: (500 MHz, CDCl₃) δ 11.17 (s, 1H), 7.82 (dd, J=7.8, 1.7 Hz, 2H), 7.53-7.46 (m, 1H), 7.42 (dd, J=8.5, 7.1 Hz, 2H), 7.32-7.27 (m, 2H), 7.01-6.95 (m, 2H), 4.32 (s, 1H), 3.86 (s, 1H), 1.33 (d, J=6.8 Hz, 6H), 1.15 (d, J=6.9 Hz, 6H) ppm. ¹³C NMR: (126 MHz, CDCl₃) δ 157.73, 140.74, 136.10, 132.94, 132.38, 129.17, 126.96, 117.98, 47.36, 45.50, 21.22, 21.07, 20.95, 20.80 ppm. Specific rotation:

$$[ α ] \frac{23}{D} = - 128.38$$

(c 1.00, CHCl₃) HRMS: Calc'd for C₁₉H₂₅BrN₃O₂S [M+H⁺] 438.0845; found 438.0852. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 4.927 min, major: 5.447 min.

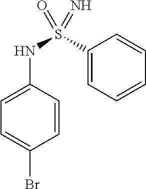 [SMILES: N=[S@](=O)(Nc1ccc(Br)cc1)c1ccccc1]

GP-14 was followed with no additional change: Purified by silica gel column chromatography using hexane/acetone (0% to 50% acetone gradient) to give the product (26 mg, 84 μmol, 84% yield) as a white a white amorphous solid.

Physical characteristics: White amorphous solid. TLC: R_{f}=0.30 (hexane/acetone, 35% acetone). ¹H NMR: (500 MHz, DMSO-d6) δ 7.93 (d, 2H), 7.62-7.53 (m, 3H), 7.37-7.26 (m, 4H), 6.93 (d, J=8.2 Hz, 2H) ppm. ¹³C NMR: (126 MHZ, DMSO-d6) & 144.87, 143.68,

131.86, 131.41, 128.88, 126.43, 124.84, 112.22 ppm. Specific rotation:

$$[ α ] \frac{23}{D} = + 262.22$$

(c 0.5, CHCl₃) HRMS: Calc'd for C₁₂H₁₂N₂OS [M+H⁺] 310.9848; found 310.9848. ee>99% HPLC Conditions: Chiralpak IC column, 70:30 n-hexane:i-PrOH, flow rate: 1 mL min-1, 25° C., UV detection wavelength: 220 nm, retention time: minor: 6.193 min, major: 6.600 min. Specific rotation:

$$[ α ] \frac{23}{D} = + 26 \; 2.22$$

(c 0.5, CHCl₃) HRMS: Calc'd for C₁₂H₁₂N₂OS [M+H⁺] 310.9848; found 310.9848. Enantiomeric excess: >99% ee HPLC Conditions: Chiralpak IC column, 70:30 n-hexane:i-PrOH, flow rate: 1 mL min-1, 25° C., UV detection wavelength: 220 nm, retention time: minor: 6.193 min, major: 6.600 min.

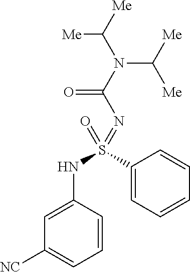 [SMILES: CC(C)N(C(=O)N=[S@](=O)(Nc1cccc(C#N)c1)c1ccccc1)C(C)C]

GP-9 was followed with no additional change: Commercially available 3-aminobenzonitrile (1.0 eq., 0.25 mmol) was used. NaHMDS (2.0 eq) was used as base. Purified by silica gel column chromatography using hexane/EtOAc (0% to 30% EtOAc gradient) to give the product (83 mg, 216 μmol, 86% yield) as a white amorphous solid.

Physical characteristics: White amorphous solid. TLC: R_{f}=0.28 (hexane/EtOAc, 25% EtOAc). ¹H NMR: (500 MHz, CDCl₃) δ 7.85 (dd, J=7.5, 1.8 Hz, 2H), 7.53 (dd, J=8.4, 6.4 Hz, 1H), 7.46 (t, J=7.7 Hz, 2H), 7.37 (d, J=2.2 Hz, 1H), 7.34-7.26 (m, 3H), 4.31 (s, 1H), 3.85 (s, 1H), 1.33 (d, J=6.9 Hz, 6H), 1.15 (d, J=6.6 Hz, 6H) ppm. ¹³C NMR: (126 MHz, CDCl₃) δ 157.54, 140.54, 138.29, 133.22, 130.25, 129.35, 127.96, 126.91, 125.36, 123.90, 118.23, 113.35, 47.42, 45.57, 21.14, 21.02, 20.88, 20.75 ppm. Specific rotation:

$$[ α ] \frac{23}{D} = - 153.17$$

(c 1.00, CHCl₃) HRMS: Calc'd for C₂₀H₂₅N₄O₂S [M+H⁺] 385.1693; found 385.1690. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 8.700 min, major: 9.327 min.

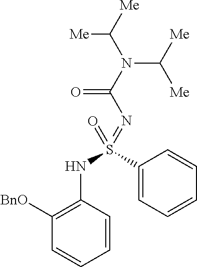 [SMILES: CC(C)N(C(=O)N=[S@](=O)(Nc1ccccc1OCc1ccccc1)c1ccccc1)C(C)C]

GP-9 was followed with no additional change: Commercially available 2-(benzyloxy) aniline (1.0 eq) was used. NaHMDS (2.0 eq) was used as base. Purified by silica gel column chromatography using hexane/EtOAc (0% to 20% EtOAc gradient) to give the product (104 mg, 223 μmol, 89% yield) as a colorless oil, which solidified to a white amorphous solid.

Physical characteristics: White amorphous solid. TLC: R_{f}=0.36 (hexane/EtOAc, 20% EtOAc). ¹H NMR: (500 MHz, CDCl₃) δ 11.16 (s, 1H), 7.82 (dd, J=7.8, 1.6 Hz, 2H), 7.60-7.53 (m, 2H), 7.46 (dd, J=8.4, 1.6 Hz, 1H), 7.44-7.35 (m, 3H), 7.35-7.27 (m, 3H), 6.96 (td, J=7.8, 1.6 Hz, 1H), 6.82 (t, J=7.4 Hz, 2H), 5.10 (d, J=12.1 Hz, 1H), 4.98 (d, J=12.2 Hz, 1H), 4.40 (s, 1H), 3.79 (s, 1H), 1.36 (dd, J=7.0, 3.6 Hz, 6H), 1.14 (dd, J=21.3, 6.8 Hz, 6H) ppm. ¹³C NMR: (126 MHz, CDCl₃) δ 157.40, 149.10, 141.41, 136.62, 132.40, 128.76, 128.58, 127.80, 127.04, 126.93, 126.79, 125.02, 122.12, 121.26, 112.33, 70.38, 47.44, 45.31, 21.24, 21.07, 21.04, 20.88 ppm. Specific rotation:

$$[ α ] \frac{23}{D} = + 6.49$$

(c 1.00, CHCl₃) HRMS: Calc'd for C₂₆H₃₂N₃O₃S [M+H⁺] 466.2159; found 466.2154. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 6.333 min, major: 7.067 min.

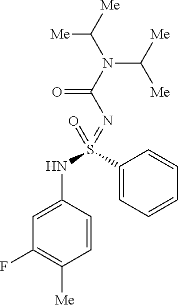 [SMILES: Cc1ccc(N[S@@](=O)(=NC(=O)N(C(C)C)C(C)C)c2ccccc2)cc1F]

GP-9 was followed with no additional change: Commercially available 3-fluoro-4-methylaniline (1.0 eq, 0.2 mmol) was used. NaHMDS (2.0 eq) was used as base. Purified by silica gel column chromatography using hexane/EtOAc (0% to 20% EtOAc gradient) to give the product (68 mg, 174 μmol, 69% yield) as a colorless oil.

Physical characteristics: Colorless oil. TLC: R_{f}=0.36 (hexane/EtOAc, 20% EtOAc). ¹H NMR: (500 MHZ, CDCl₃) δ 7.83 (dd, J=7.6, 1.8 Hz, 2H), 7.49 (t, J=7.4 Hz, 1H), 7.42 (t, J=7.7 Hz, 2H), 6.96 (t, J=8.3 Hz, 1H), 6.82 (dd, J=10.8, 2.2 Hz, 1H), 6.75 (dd, J=8.1, 2.2 Hz, 1H), 4.32 (s, 1H), 3.98-3.77 (m, 1H), 2.13 (s, 3H), 1.33 (d, J=6.9 Hz, 6H), 1.15 (d, J=6.9 Hz, 6H) ppm. ¹³C NMR: (126 MHZ, CDCl₃) δ 162.29, 160.34, 157.86, 140.92, 135.96 (d, J=10.2 Hz), 132.93, 131.89 (d, J=6.3 Hz), 129.19, 127.08, 121.41 (d, J=17.4 Hz), 117.39 (d, J=3.4 Hz), 109.12 (d, J=25.8 Hz), 47.41, 45.56, 21.30, 21.16, 21.04, 20.90, 14.21 (d, J=3.2 Hz) ppm. 19F NMR: (471 MHz, CDCl₃) δ −115.20 ppm. Specific rotation:

$$[ α ] \frac{23}{D} = - 135.45$$

(c 1.00, CDCl₃) HRMS: Calc'd for C₂₀H₂₇FN₃O₂S [M+H⁺] 392.1803; found 392.1803. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 5.120 min, major: 5.620 min.

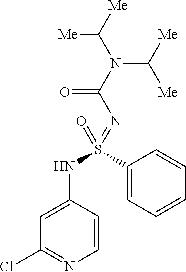 [SMILES: CC(C)N(C(=O)N=[S@](=O)(Nc1ccnc(Cl)c1)c1ccccc1)C(C)C]

GP-9 was followed with additional changes mentioned: Commercially available 2-chloropyridin-4-amine (1.0 eq, 0.2 mmol) and NaHMDS (2.0 eq) were used. The reaction was quenched after stirring at room temperature for 1.5 hours. Purified by silica gel column chromatography using hexane/EtOAc (0% to 50% EtOAc gradient) to give the product (71 mg, 0.180 mmol, 90% yield) as a clear colorless oil.

Physical characteristics: Clear colorless oil. TLC: R_{f}=0.36 (hexane/EtOAc, 40% EtOAc). ¹H NMR: (500 MHz, CDCl₃) δ 12.54 (s, 1H), 8.10 (d, J=5.6 Hz, 1H), 7.94-7.89 (m, 2H), 7.61-7.55 (m, 1H), 7.53-7.48 (m, 2H), 7.03 (d, J=2.0 Hz, 1H), 6.91 (dd, J=5.6, 2.0 Hz, 1H), 4.30 (s, 1H), 3.84 (s, 1H), 1.33 (d, J=3.8 Hz, 6H), 1.14 (d, J=3.5 Hz, 6H) ppm. ¹³C NMR: (126 MHz, CDCl₃) δ 157.1, 152.3, 150.2, 147.0, 140.3, 133.4, 129.4, 126.8, 113.0, 112.3, 47.4, 45.6, 21.0, 20.9, 20.8, 20.6 ppm. Specific rotation:

$$[ α ] \frac{22.8}{D} = - 65.23$$

(c 1.00, CHCl₃) HRMS: Calc'd for C₁₈H₂₃ClN₄O₂SNa [M+Na⁺] 417.1122; found 417.1122. Enantiomeric excess: >99% ee. HPLC Conditions: Daicel Chiralpak IC column, 70:30 n-hexane:i-PrOH, flow rate: 1 mL/min, 25° C., UV detection wavelength: 254 nm, retention time: minor: 6.97 min, major: 12.84 min.

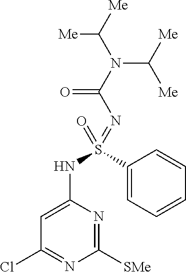 [SMILES: CSc1nc(Cl)cc(N[S@@](=O)(=NC(=O)N(C(C)C)C(C)C)c2ccccc2)n1]

GP-9 was followed with additional changes mentioned: Commercially available 6-chloro-2-(methylthio)pyrimidin-4-amine (1.0 eq, 0.20 mmol) and NaHMDS (2.0 eq) were used. The reaction was quenched after stirring at room temperature for 1.5 hours. Purified by silica gel column chromatography using hexane/EtOAc (0% to 60% EtOAc gradient) to give the product (76 mg, 0.172 mmol, 86% yield) as a white amorphous solid.

Physical characteristics: White amorphous solid. TLC: R_{f}=0.41 (hexane/EtOAc, 40% EtOAc). ¹H NMR: (500 MHZ, CDCl₃) δ 9.06 (s, OH), 8.00-7.95 (m, 2H), 7.60-7.57 (m, 1H), 7.54-7.50 (m, 2H), 6.59 (s, 1H), 4.39-4.16 (m, 1H), 4.00-3.78 (m, 1H), 2.36 (s, 3H), 1.35-1.12 (m, 12H) ppm. ¹³C NMR: ¹³C NMR (126 MHz, CDCl₃) δ 171.49, 164.17, 160.33, 140.98, 133.39, 129.29, 128.97, 127.07, 107.77, 47.39, 20.89, 20.82, 19.30, 14.07 ppm. Specific rotation:

$$[ α ] \frac{23.3}{D} = - 4.62$$

(c 1.00, CDCl₃) HRMS: Calc'd for C₁₆H₂₄ClN₅O₂S₂Na [M+Na⁺] 464.0952; found 464.0957. Enantiomeric excess: >99% ee. HPLC Conditions: Daicel Chiralpak IC column, 70:30 n-hexane:i-PrOH, flow rate: 1 mL/min, 25° C., UV detection wavelength: 220 nm, retention time: minor: 5.42 min, major: 8.53 min.

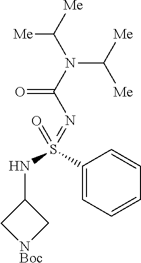 [SMILES: CC(C)N(C(=O)N=[S@](=O)(NC1CN(C(=O)OC(C)(C)C)C1)c1ccccc1)C(C)C]

GP-11 (LiBr) was followed with no addition change: Commercially available tert-butyl 3-aminoazetidine-1-carboxylate (1.0 eq. 0.1 mmol) was used. Heated the reaction at 60° C. for 13 hours. Purified by silica gel column chromatography using hexane/EtOAc (0% to 60% EtOAc gradient) to give the product (34.6 mg, 78.9 μmol, 79% yield) as a clear colorless oil.

Physical characteristics: Clear colorless oil. TLC: R_{f}=0.41 (hexane/EtOAc, 50% EtOAc). ¹H NMR: (500 MHz, CDCl₃) δ 8.79 (d, J=8.8 Hz, 1H), 7.92-7.87 (m, 2H), 7.61-7.55 (m, 1H), 7.53-7.48 (m, 2H), 4.37-4.19 (m, 1H), 4.20-4.13 (m, 1H), 4.08-4.00 (m, 1H), 3.95 (dd, J=9.1, 5.6 Hz, 1H), 3.91-3.79 (m, 1H), 3.78-3.72 (m, 1H), 3.54 (dd, J=9.3, 5.7 Hz, 1H), 1.39 (s, 9H), 1.31 (d, J=6.8 Hz, 6H), 1.15 (d, J=6.9 Hz, 6H) ppm. ¹³C NMR: ¹³C NMR (126 MHz, CDCl₃) δ 157.9, 155.8, 141.0, 133.0, 129.2, 126.9, 79.9, 57.4, 47.2, 45.3, 41.5, 28.3, 21.1, 21.0, 20.8, 20.8 ppm. Specific rotation:

$$[ α ] \frac{23}{D} = - 26.86$$

(c 1.00, CHCl₃) HRMS: Calc'd for C₂₁H₃₄N₄O₄SNa [M+Na⁺] 461.2193; found 461.2195. Enantiomeric excess: >99% ee. HPLC Conditions: Daicel Chiralpak IC column, 70:30 n-hexane:i-PrOH, flow rate: 1 mL/min, 25° C., UV detection wavelength: 254 nm, retention time: minor: 10.09 min, major: 12.91 min.

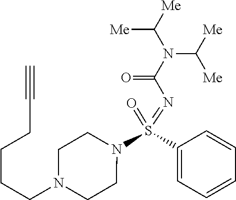 [SMILES: C#CCCCCN1CCN([S@@](=O)(=NC(=O)N(C(C)C)C(C)C)c2ccccc2)CC1]

GP-11 (LiBr) was followed with no additional change: 1-(hex-5-yn-1-yl) piperazine (1.1 eq. 0.220 mmol) was used (prepared from the procedure below). Heated the reaction at 70° C. for 13 hours. Purified by silica gel column chromatography using hexane/EtOAc (0% to 60% EtOAc gradient) to give the product (85 mg, 0.196 mmol, 98% yield) as a clear light-yellow oil.

Physical characteristics: Clear light-yellow oil. TLC: R_{f}=0.2 (hexane/EtOAc, 60% EtOAc). ¹H NMR: (500 MHz, CDCl₃) δ 7.84-7.79 (m, 2H), 7.58-7.48 (m, 3H), 4.23 (s, 1H), 3.88 (s, 1H), 3.20-3.06 (m, 4H), 2.56-2.47 (m, 4H), 2.34 (t, J=7.0 Hz, 2H), 2.17 (td, J=6.8, 2.6 Hz, 2H), 1.92 (t, J=2.6 Hz, 1H), 1.59-1.44 (m, 4H), 1.30-1.20 (m, 12H) ppm. ¹³C NMR: (126 MHz, CDCl₃) δ 157.20, 136.73, 132.47, 128.98, 127.59, 84.16, 68.52, 57.48, 52.33, 47.11, 45.80, 45.14, 26.20, 25.77, 21.51, 20.77, 18.27 ppm. Specific rotation:

$$[ α ] \frac{23.3}{D} = - 37.98$$

(c 1.00, CHCl₃) HRMS: Calc'd for C₂₃H₃₇N₄O₂S [M+H⁺] 433.2632; 433.2636. Enantiomeric excess: Unable to separate enantiomers.

### Synthesis of 1-(hex-5-yn-1-yl) piperazine

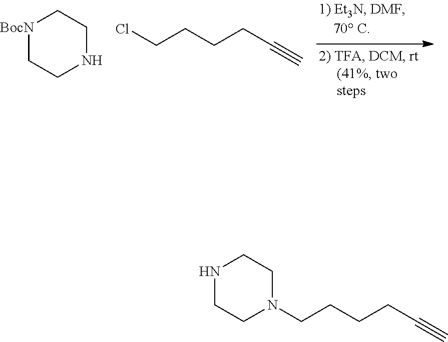

### Step 1:

In a 200 mL round-bottomed flask equipped with a stir bar was tert-butyl piperazine-1-carboxylate (2.55 g, 21.9 mmol, 1 eq.) in DMF (60 mL, 0.36 M). 6-chlorohex-1-yne (4.48 g, 24.1 mmol, 1.1 eq.) and Et₃N (3.66 mL, 26.3 mmol, 1.2 eq.) were added then the reaction vessel was capped with a septum and heated to 70° C. for 21 hours. The reaction mixture was cooled to room temperature and DMF was removed under vacuum to give a crude residue that was taken up in EtOAc (100 mL) and half saturated aqueous NaCl (150 mL). The aqueous layer was extracted with EtOAc (100 mL×3), combined organic layers were dried over Na₂SO₄, filtered and concentrated. Further purification by silica gel column chromatography using DCM/MeOH (0% to 3% MeOH gradient) gave tert-butyl 4-(hex-5-yn-1-yl) piperazine-1-carboxylate (2.49 g, 9.35 mmol, 43% yield) as a colorless foam that was used in the next step.

Physical characteristics: Colorless foam. TLC: R_{f}=0.38 (DCM/MeOH, 5% MeOH). ¹H NMR: (500 MHz, CDCl₃) δ 3.55-3.39 (m, 4H), 2.54-2.36 (m, 6H), 2.22 (td, J=6.9, 2.6 Hz, 2H), 1.94 (t, J=2.6 Hz, 1H), 1.69-1.61 (m, 2H), 1.58-1.52 (m, 2H), 1.45 (s, 9H). ¹³C NMR: (126 MHZ, CDCl₃) δ 154.68, 84.14, 79.75, 68.59, 57.95, 52.90, 42.85, 28.42, 26.26, 25.47, 18.28. HRMS: Calc'd for C₁₅H₂₇N₂O₂ [M+H⁺] 267.2067; found 267.2063.

### Step 2:

In a 100 mL septum capped round-bottomed flask equipped with a stir bar and argon balloon was tert-butyl 4-(hex-5-yn-1-yl) piperazine-1-carboxylate (2.49 g, 9.35 mmol, 1 eq.) in DCM (9.4 mL, 1.0 M) was added TFA (9.37 mL, 122 mmol, 13 eq.) at room temperature. The reaction mixture stirred at room temperature for 3 hours at which time the solvents were removed under reduced pressure to give a crude oil that was taken up in DCM (250 mL), washed with saturated Na₂CO₃ (100 mL×3), dried over Na₂SO₄, filtered and concentrated to give 1-(hex-5-yn-1-yl) piperazine (1.48 g, 8.90 mmol, 95% yield) as an off-white amorphous solid that was used without further purification.

Physical characteristics: Off-white amorphous solid. TLC: R_{f}=0.18 (DCM/MeOH, 5% MeOH). ¹H NMR: (500 MHz, CDCl₃) δ 2.88 (t, J=4.8 Hz, 4H), 2.50-2.34 (m, 4H), 2.33-2.28 (m, 2H), 2.19 (td, J=6.8, 2.6 Hz, 2H), 2.13 (s, 1H), 1.92 (t, J=2.5 Hz, 1H), 1.64-1.49 (m, 4H) ppm. ¹³C NMR: (126 MHz, CDCl₃) δ 84.34, 68.40, 58.64, 54.44, 46.01, 26.45, 25.71, 18.35. HRMS: Calc'd for C₁₀H₁₉N₂ [M+H⁺] 167.1543; found 167.1538.

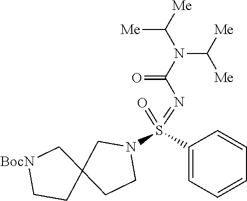 [SMILES: CC(C)N(C(=O)N=[S@@](=O)(c1ccccc1)N1CCC2(CCN(C(=O)OC(C)(C)C)C2)C1)C(C)C]

GP-9 (NaHMDS) was followed with no additional change: Commercially available tert-butyl 2,7-diazaspiro[4.4]nonane-2-carboxylate (1.0 eq, 0.2 mmol) and NaHMDS (1.0 eq) were used. Purified by silica gel column chromatography using hexane/EtOAc (0% to 40% EtOAc gradient) to give an inseparable mixture of diastereomers (92 mg, 187 mmol, 93% yield) as a colorless foam.

Physical characteristics: Colorless foam TLC: R_{f}=0.58 (hexane/EtOAc, 60% EtOAc). ¹H NMR: (500 MHZ, CDCl₃) δ 7.89 (d, J=6.9 Hz, 2H), 7.61-7.49 (m, 3H), 4.31-4.06 (m, 1H), 4.03-3.76 (m, 1H), 3.60-3.41 (m, 1H), 3.39-2.98 (m, 7H), 1.89-1.75 (m, 3H), 1.69-1.63 (m, 1H), 1.42 (s, 9H), 1.30-1.21 (m, 12H) ppm. ¹³C NMR: (126 MHZ, CDCl₃) δ 157.46, 154.43, 138.54, 132.52, 129.09, 127.10, 79.50, 56.22, 56.12, 54.80, 54.17, 49.00, 48.12, 47.20, 47.08, 45.12, 45.04, 44.67, 35.08, 34.94, 34.84, 34.27, 28.48, 21.47, 20.78 ppm. Note: The spirocyclic amine nucleophile used was racemic. HRMS: Calc'd for C₂₅H₄₁N₄O₄S [M+H⁺] 493.2843; found 493.2849.

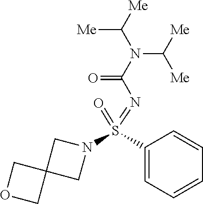 [SMILES: CC(C)N(C(=O)N=[S@@](=O)(c1ccccc1)N1CC2(COC2)C1)C(C)C]

GP-11 (NaI) was followed with addition changes mentioned: Commercially 2-oxa-6-azaspiro[3.3]heptane oxalate (1.0 eq., 0.1 mmol) was used. The equivalents of Et₃N were increased from 2 to 6 equivalents and the reaction was heated to 60° C. for 24 hours; no further modifications were made. Purified by silica gel column chromatography using hexane/EtOAc (0% to 40% EtOAc gradient) to give the product (30 mg, 82 μmol, 82% yield) as a white amorphous solid.

Physical characteristics: White amorphous solid. TLC: R_{f}=0.32 (hexane/EtOAc, 60% EtOAc). ¹H NMR: (500 MHz, CDCl₃) δ 7.90-7.85 (m, 2H), 7.61-7.56 (m, 1H), 7.54-7.49 (m, 2H), 4.71 (d, J=7.2 Hz, 2H), 4.67 (d, J=7.2 Hz, 2H), 4.18 (d, J=8.4 Hz, 2H), 4.13 (s, 1H), 3.98 (d, J=8.4 Hz, 2H), 3.93 (s, 1H), 1.29-1.20 (m, 12H) ppm. ¹³C NMR: (126 MHz, CDCl₃) δ 157.74, 137.97, 132.83, 129.07, 127.57, 80.88, 59.73, 47.03, 45.20, 37.36, 21.46, 20.75 ppm. Specific rotation:

$$[ α ] \frac{23.2}{D} = - 5.21$$

(c 0.90, CHCl₃) HRMS: Calc'd for C₁₈H₂₇N₃O₃S [M+Na⁺] 388.1665; found 388.1666. Enantiomeric excess: >99% ee.

Note: When LiBr was used, 98% ee was obtained HPLC Conditions: Daicel Chiralpak IC column, 70:30 n-hexane:i-PrOH, flow rate: 1 mL/min, 25° C., UV detection wavelength: 220 nm, retention time: minor: 31.32 min, major: 53.79 min.

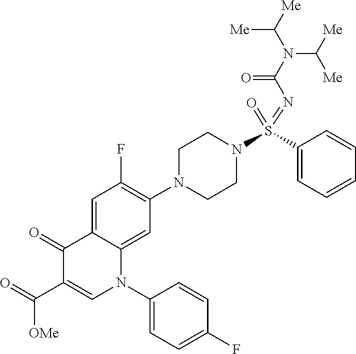 [SMILES: COC(=O)c1cn(-c2ccc(F)cc2)c2cc(N3CCN([S@@](=O)(=NC(=O)N(C(C)C)C(C)C)c4ccccc4)CC3)c(F)cc2c1=O]

GP-11 (LiBr) was followed with no additional change: Commercially available sarafloxacin HCl salt was used to prepare the methyl ester analog of sarafloxacin,⁴ which was used as the secondary amine nucleophile (1 eq., 0.1 mmol). The reaction was heated to 70° C. for 10 hours. Purified by silica gel column chromatography using hexanes/acetone (0% to 50% acetone gradient) to give the product (54 mg, 81.1 μmol, 81% yield) as a light-yellow oil.

Physical characteristics: Light-yellow oil. TLC: R_{f}=0.48 (hexanes/acetone, 50% acetone). ¹H NMR: (500 MHZ, CDCl₃) δ 8.39 (s, 1H), 8.03 (d, J=12.9 Hz, 1H), 7.87-7.82 (m, 2H), 7.61-7.57 (m, 1H), 7.56-7.51 (m, 2H), 7.44-7.39 (m, 2H), 7.33 (t, J=8.3 Hz, 2H), 6.23 (d, J=6.9 Hz, 1H), 4.28-4.12 (m, 1H), 3.97-3.82 (m, 4H), 3.24 (dd, J=6.5, 3.5 Hz, 4H), 3.09 (dd, J=6.4, 3.7 Hz, 4H), 1.31-1.18 (m, 12H) ppm. ¹³C NMR: (126 MHz, CDCl₃) δ 173.00 (d, J=2.1 Hz), 166.09, 164.06, 162.05, 156.78, 154.29, 152.31, 148.55, 144.13 (d, J=10.8 Hz), 138.06, 136.97, 136.51 (d, J=3.5 Hz), 132.77, 129.28 (d, J=9.0 Hz), 129.13, 127.37, 123.33 (d, J=7.0 Hz), 117.80 (d, J=23.2 Hz), 113.37 (d, J=23.2 Hz), 110.69, 106.23 (d, J=2.7 Hz), 52.20, 49.30 (d, J=4.1 Hz), 47.18, 45.64, 45.27, 21.50, 20.75, 20.68 ppm. 19F NMR: (471 MHz, CDCl₃) δ −108.84, −123.42 ppm. HRMS: Calc'd for C₃₄H₃₈F₂N₅O₅S [M+H⁺] 666.2556; found 666.2560. Enantiomeric excess: Unable to separate enantiomers.

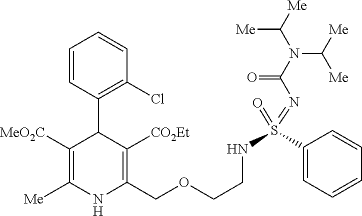 [SMILES: CCOC(=O)C1=C(COCCN[S@@](=O)(=NC(=O)N(C(C)C)C(C)C)c2ccccc2)NC(C)=C(C(=O)OC)C1c1ccccc1Cl]

GP-11 (LiBr) was followed with additional changes mentioned: Ph-sulfonimidoyl fluoride (1.1 eq., 0.11 mmol) was used. Commercially available rac-amlodipine (1 eq., 0.1 mmol) was used. The reaction was heated at 70° C. for 13 hours. Purified by silica gel column chromatography using hexane/EtOAc (0% to 35% EtOAc gradient) to give an inseparable mixture of diastereomers (47 mg, 69.6 μmol, 70% yield) as a light-yellow oil. Physical characteristic: Light-yellow oil. TLC: R_{f}=0.38 (hexane/EtOAc, 40% EtOAc). ¹H NMR: (500 MHZ, CDCl₃) δ 8.91-8.75 (m, 1H), 7.94 (d, J=7.9 Hz, 2H), 7.61-7.55 (m, 1H), 7.54-7.39 (m, 4H), 7.22 (d, J=7.9 Hz, 1H), 7.14 (q, J=7.5 Hz, 1H), 7.04 (t, J=7.6 Hz, 1H), 5.41 (d, J=2.1 Hz, 1H), 4.79-4.59 (m, 2H), 4.32-4.10 (m, 1H), 4.09-3.98 (m, 2H), 3.98-3.79 (m, 1H), 3.73-3.64 (m, 1H), 3.62 (s, 3H), 3.58-3.51 (m, 1H), 3.35-3.23 (m, 1H), 3.13-3.03 (m, 1H), 2.43 (d, J=2.6 Hz, 3H), 1.27 (s, 6H), 1.21-1.12 (m, 9H) ppm. ¹³C NMR: (126 MHz, CDCl₃) δ 168.15, 167.25, 158.13, 158.09, 146.06, 146.03, 145.20, 145.16, 145.01, 144.93, 140.85, 140.79, 132.70, 132.69, 132.26, 132.17, 131.54, 131.51, 129.16, 129.13, 129.11, 127.31, 127.29, 127.00, 126.91, 103.70, 103.68, 101.64, 101.56, 69.56, 69.49, 67.98, 67.93, 59.79, 59.78, 50.74, 46.92, 45.27, 41.86, 37.04, 36.90, 29.72, 21.19, 21.12, 20.72, 19.35, 19.32, 14.29 ppm. Note: The primary amine nucleophile used was racemic. HRMS: Calc'd for C₃₃H₄₄ClN₄O₇S [M+H⁺] 675.2614; found 675.2611.

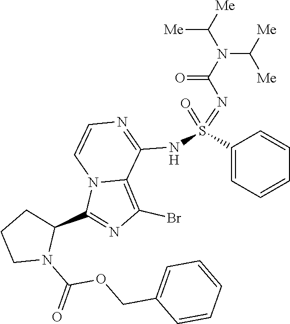 [SMILES: CC(C)N(C(=O)N=[S@](=O)(Nc1nccn2c([C@@H]3CCCN3C(=O)OCc3ccccc3)nc(Br)c12)c1ccccc1)C(C)C]

GP-9 was followed with additional changed mentioned: Reaction scale changed to 0.20 mmol. Commercially available benzyl (2S)-2-{8-amino-1-bromoimidazo[1,5-a]pyrazin-3-yl}pyrrolidine-1-carboxylate (0.20 mmol, 1.0 eq) and NaHMDS (2.0 eq) were used. Purified by silica gel column chromatography using hexane/acetone (0% to 45% acetone gradient) to give the product (110 mg, 161 μmol, 80% yield) as a white amorphous solid.

Physical characteristics: White amorphous solid. TLC: R_{f}=0.30 (hexane/acetone, 30% acetone). ¹H NMR: (500 MHZ, CDCl₃) δ 7.83 (dd, J=7.6, 1.8 Hz, 2H), 7.49 (t, J=7.4 Hz, 1H), 7.42 (t, J=7.7 Hz, 2H), 6.96 (t, J=8.3 Hz, 1H), 6.82 (dd, J=10.8, 2.2 Hz, 1H), 6.75 (dd, J=8.1, 2.2 Hz, 1H), 4.32 (s, 1H), 3.98-3.77 (m, 1H), 2.13 (s, 3H), 1.33 (d, J=6.9 Hz, 6H), 1.15 (d, J=6.9 Hz, 6H) ppm. ¹³C NMR: (126 MHz, CDCl₃) δ 162.29, 160.34, 157.86, 140.92, 135.96 (d, J=10.2 Hz), 132.93, 131.89 (d, J=6.3 Hz), 129.19, 127.08, 121.41 (d, J=17.4 Hz), 117.39 (d, J=3.4 Hz), 109.12 (d, J=25.8 Hz), 47.41, 45.56, 21.30, 21.16, 21.04, 20.90, 14.21 (d, J=3.2 Hz) ppm. Specific rotation:

$$[ α ] \frac{23}{D} = - 19.56$$

### (c 1.00, CHCl₃)

HRMS: Calc'd for C₃₁H₃₇BrN₇O₄S [M+H⁺] 682.1806; found 682.1810 Diastereomeric Excess: >99 de, determined by ¹HNMR.

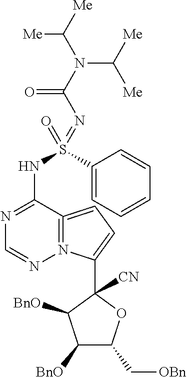

GP-9 was followed with no additional change: Commercially available (2R,3R,4R,5R)-2-(4-aminopyrrolo[2,1-f][1,2,4]triazin-7-yl)-3,4-bis (benzyloxy)-5-((benzyloxy)methyl)tetrahydrofuran-2-carbonitrile (0.1 mmol, 1 eq.) and NaHMDS (2.0 eq) were used. Purified by silica gel column chromatography using hexane/EtOAc (0% to 35% EtOAc gradient) to give the product (66 mg, 79.7 μmol, 80% yield) as a light-yellow amorphous solid.

Physical characteristics: light-yellow amorphous solid. TLC: R_{f}=0.67 (hexane/EtOAc, 40% EtOAc). ¹H NMR: (500 MHZ, CDCl₃) δ 8.15 (d, J=7.6 Hz, 2H), 7.63-7.53 (m, 3H), 7.49 (s, 1H), 7.33-7.23 (m, 15H), 6.92-6.84 (m, 2H), 4.84 (s, 2H), 4.65 (d, J=4.9 Hz, 1H), 4.57-4.46 (m, 4H), 4.37 (d, J=12.0 Hz, 1H), 4.23 (s, 1H), 4.02 (t, J=5.8 Hz, 1H), 3.93 (s, 1H), 3.76 (dd, J=11.0, 3.5 Hz, 1H), 3.60 (dd, J=11.0, 3.9 Hz, 1H), 1.34 (dd, J=12.3, 6.8 Hz, 6H), 1.26-1.18 (m, 6H) ppm. Specific rotation:

$$[ α ] \frac{22.7}{D} = + 62.96$$

(c 1.00, CHCl₃) HRMS: Calc'd for C₄₈H₅₀N₇O₆S [M+H⁺] 828.3538; found 828.3529. Diastereomeric excess: >99% de (determined by ¹H NMR)

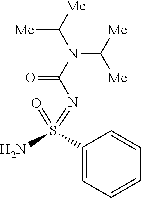 [SMILES: CC(C)N(C(=O)N=[S@@](N)(=O)c1ccccc1)C(C)C]

GP-10 was followed with no additional change: Commercially available NH₄Cl (3.0 eq) was used as a nitrogen source. i-PrMgCl—LiCl (6.0 eq) was used as base. NH₄Cl and i-PrMgClLiCl were mixed at room temperature in THF (2.5 mL) and stirred the mixture for 30 min, Ph-sulfonimidoyl fluoride (0.25 mmol, 1 eq.) in THF (0.5 mL) was added to the reaction slowly. Purified by silica gel column chromatography using hexane/EtOAc (0% to 60% EtOAc gradient) to give the product (58 mg, 204 μmol, 82% yield) as a white amorphous solid.

Physical characteristics: White amorphous solid. TLC: R_{f}=0.3 (hexane/EtOAc, 50% EtOAc). ¹H NMR: (500 MHz, CDCl₃) δ 7.95 (dd, J=7.5, 1.7 Hz, 2H), 7.58-7.52 (m, 1H), 7.49 (dd, J=8.4, 6.7 Hz, 2H), 6.50 (s, 2H), 4.29 (s, 1H), 3.81 (s, 1H), 1.29-1.15 (m, 12H) ppm. ¹³C NMR: (126 MHz, CDCl₃) δ 158.34, 143.27, 132.66, 129.07, 126.36, 47.23, 45.21, 21.26, 21.17, 20.91 ppm. Specific rotation:

$$[ α ] \frac{23}{D} = - 24.24$$

(c 1.00, CHCl₃) HRMS: Calc'd for C₁₃H₂₂N₃O₂S [M+H⁺] 284.1427; found 284.1427. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 7.673 min, minor: 8.6734 min.

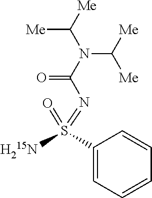 [SMILES: CC(C)N(C(=O)N=[S@@]([15NH2])(=O)c1ccccc1)C(C)C]

GP-10 was followed with no additional change: Commercially available 15NH₄Br (3.0 eq) was used as a nitrogen source. i-PrMgCl—LiCl (6.0 eq) was used as base. 15NH₄Br and i-PrMgClLiCl were mixed at room temperature in THF (2.5 mL) and stirred the mixture for 30 min, Ph-sulfonimidoyl fluoride (0.25 mmol, 1 eq.) in THF (0.5 mL) was added to the reaction slowly. Purified by silica gel column chromatography using hexane/EtOAc (0% to 60% EtOAc gradient) to give the product (58 mg, 204 μmol, 82% yield) as a white solid.

Physical characteristics: White amorphous solid. TLC: R_{f}=0.3 (hexane/EtOAc, 50% EtOAc). ¹H NMR: (500 MHz, CDCl₃) δ 7.99-7.90 (m, 2H), 7.54 (t, J=4.6 Hz, 1H), 7.48 (td, J=7.9, 7.5, 2.2 Hz, 2H), 6.53 (d, J=53.6 Hz, 2H), 4.28 (s, 1H), 3.80 (s, 1H), 1.21 (dd, J=32.2, 7.5 Hz, 12H). ppm. ¹³C NMR: (126 MHZ, CDCl₃) δ 158.34, 143.27, 132.66, 129.07, 126.36, 47.26, 45.21, 21.26, 21.17, 20.91 ppm. Specific rotation:

$$[ α ] \frac{23}{D} = - 22 \text{.10}$$

(c 1.00, CHCl₃) HRMS: Calc'd for C₁₃H₂₂N₂¹⁵NO₂S [M+H⁺] 285.1398; found 285.1398. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 7.387 min, major: 8.340 min.

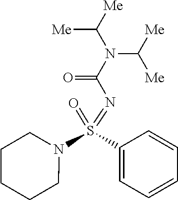 [SMILES: CC(C)N(C(=O)N=[S@@](=O)(c1ccccc1)N1CCCCC1)C(C)C]

GP-12 was followed with no additional change: Commercially available piperidine (1.0 eq) was used. i-PrMgCl—LiCl (1.1 eq) was used as base. i-PrMgCl—LiCl was added to a solution of piperidine in THF (2.5 mL) under −20° C. and stirred the mixture for 30 min, Ph-sulfonimidoyl fluoride (0.25 mmol, 1 eq.) in THF (0.5 mL) was added to the reaction slowly. Purified by silica gel column chromatography using hexane/EtOAc (0% to 60% EtOAc gradient) to give the product (76 mg, 216 μmol, 86% yield) as a white amorphous solid.

Physical characteristics: White amorphous solid. TLC: R_{f}=0.3 (hexane/EtOAc, 50% EtOAc). ¹H NMR: (500 MHz, CDCl₃) δ 7.85 (dd, J=7.2, 1.8 Hz, 2H), 7.58-7.52 (m, 1H), 7.50 (dd, J=8.3, 6.5 Hz, 2H), 4.21 (s, 1H), 3.92 (s, 1H), 3.20-2.99 (m, 4H), 1.61 (p, J=5.6 Hz, 4H), 1.50-1.40 (m, 2H), 1.34-1.18 (m, 12H) ppm. ¹³C NMR: (126 MHZ, CDCl₃) δ 157.63, 138.36, 132.33, 128.95, 127.56, 46.99, 46.70, 45.19, 25.47, 23.82, 21.63, 20.91, 20.86 ppm. Specific rotation:

$$[ α ] \frac{23}{D} = - 26.24$$

(c 1.00, CHCl₃) HRMS: Calc'd for C₁₈H₃₀N₃O₂S [M+H⁺] 352.2053; found 352.2047. Enantiomeric excess: >99% ee. HPLC Conditions: Daicel Chiralpak IA column, 98:02 n-hexane:i-PrOH, flow rate: 1 mL min-1, 25° C., UV detection wavelength: 220 nm, retention time: minor: 10.77 min, major: 11.88 min.

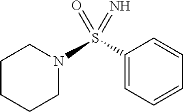 [SMILES: N=[S@@](=O)(c1ccccc1)N1CCCCC1]

GP-13 was followed with no additional change: Purified by silica gel column chromatography using hexane/acetone (0% to 50% acetone gradient) to give the product (20.5 mg, 91.3 μmol, 91% yield) as a white amorphous solid.

Physical characteristics: White amorphous solid. TLC: R_{f}=0.28 (hexane/acetone, 50% acetone). ¹H NMR: (500 MHZ, CDCl₃) § 7.91-7.84 (m, 2H), 7.58-7.53 (m, 1H), 7.53-7.48 (m, 2H), 2.98 (t, J=5.5 Hz, 4H), 2.44 (s, 1H), 1.68-1.55 (m, 4H), 1.42-1.29 (m, 2H) ppm. ¹³C NMR: (126 MHz, CDCl₃) δ 136.35, 132.27, 128.79, 128.09, 48.06, 25.77, 23.75 ppm. Specific rotation:

$$[ α ] \frac{23}{D} = + 28. 98$$

(c 1.00, CHCl₃) HRMS: Calc'd for C₁₁H₁₇N₂OS [M+H⁺] 225.1056; found 225.1056. Enantiomeric excess: >99% ee. HPLC Conditions: Chiralpak IB column, 95:05 n-hexane:i-PrOH, flow rate: 1 mL min-1, 25° C., UV detection wavelength: 220 nm, retention time: minor: 27.053 min, major: 29.040 min.

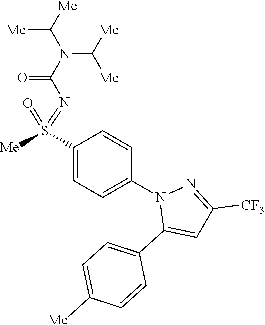 [SMILES: Cc1ccc(-c2cc(C(F)(F)F)nn2-c2ccc([S@](C)(=O)=NC(=O)N(C(C)C)C(C)C)cc2)cc1]

GP-12 was followed with no additional change: Commercially available MeMgCl (3.0 M in THF, used without titration, 0.5 mmol, 2.0 eq) was used. Purified by silica gel column chromatography using hexane/EtOAc (0% to 50% EtOAc gradient) to give the product (100 mg, 197 μmol, 79% yield) as a white amorphous solid.

Physical characteristics: White amorphous solid. TLC: R_{f}=0.38 (hexane/EtOAc, 50% EtOAc). ¹H NMR: (500 MHz, CDCl₃) δ 7.93 (d, J=8.7 Hz, 2H), 7.53 (d, J=8.7 Hz, 2H), 7.18 (d, J=8.0 Hz, 2H), 7.13 (d, J=8.2 Hz, 2H), 6.74 (s, 1H), 4.44-3.67 (m, 2H), 3.33 (s, 3H), 2.38 (s, 3H), 1.24 (t, J=6.4 Hz, 12H). ppm. ¹³C NMR: (126 MHZ, CDCl₃) δ 158.87, 145.37, 144.29 (q, J=38.5 Hz), 143.09, 140.02, 139.94, 129.91, 128.87, 128.46, 125.83, 125.69, 121.15 (q, J=269.2 Hz), 106.56 (d, J=2.0 Hz), 45.01, 21.44, 21.16 ppm. 19F NMR: (471 MHz, CDCl₃) δ −62.49. ppm. Specific rotation: (c 1.00, CHCl₃)

$$[ α ] \frac{23}{D} = - 18 \text{.22}$$

HRMS: Calc'd for C₂₅H₂₉F₃N₄NaO₂S [M+Na⁺] 529.1856; found 529.1854. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 12.940 min, minor: 22.473 min.

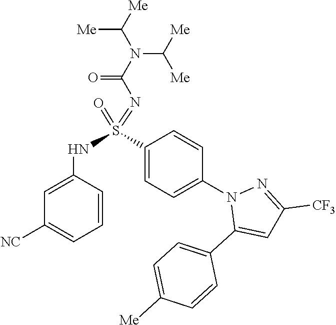 [SMILES: Cc1ccc(-c2cc(C(F)(F)F)nn2-c2ccc([S@](=O)(=NC(=O)N(C(C)C)C(C)C)Nc3cccc(C#N)c3)cc2)cc1]

GP-12 was followed with no additional change: Commercially available 3-aminobenzonitrile (0.25 mmol, 1.0 eq) was used. NaHMDS (2.0 eq) was used as base. Purified by silica gel column chromatography using hexane/EtOAc (0% to 30% EtOAc gradient) to give the product (115 mg, 189 μmol, 75% yield) as a white amorphous solid. Physical characteristics: White amorphous solid. TLC: R_{f}=0.4 (hexane/EtOAc, 35% EtOAc). ¹H NMR: (500 MHz, CDCl₃) δ 7.83 (d, J=8.7 Hz, 2H), 7.43-7.37 (m, 3H), 7.37-7.29 (m, 3H), 7.12 (d, J=7.9 Hz, 2H), 7.04-6.98 (m, 2H), 6.71 (s, 1H), 4.31 (s, 1H), 3.86 (s, 1H), 2.38 (s, 3H), 1.32 (t, J=5.8 Hz, 6H), 1.16 (d, J=6.8 Hz, 6H) ppm. ¹³C NMR: (126 MHz, CDCl₃) δ 157.36, 145.38, 144.32 (q, J=38.6 Hz), 142.74, 140.05, 139.91, 138.07, 130.42, 129.80, 128.78, 128.23, 127.90, 125.70, 125.48, 124.02, 121.10 (q, J=269.1 Hz), 118.09, 113.56, 106.46 (d, J=2.3 Hz), 47.51, 45.71, 21.44, 20.94 (dd, J=39.0, 20.4 Hz) ppm. 19F NMR: (471 MHz, CDCl₃) δ −62.52. ppm. Specific rotation:

$$[ α ] \frac{23}{D} = - 171.85$$

(c 1.00, CHCl₃) HRMS: Calc'd for C₃₁H₃₂N₆O₂S [M+H⁺] 609.2254; found 609.2254. Enantiomeric excess: >99% ee. HPLC Conditions: Daicel Chiralpak IA column, 90:10 n-hexane:i-PrOH, flow rate: 1 mL min-1, 25° C., UV detection wavelength: 220 nm, retention time: minor: 11.987 min, major: 16.800 min.
Applying the Chiral Bifunctional t-BuSF Sulfonimidoyl Transfer Reagent to Pharmaceutically Relevant Targets and Intermediates.

### Asymmetric Synthesis of a Celebrex Sulfoximine Analog.

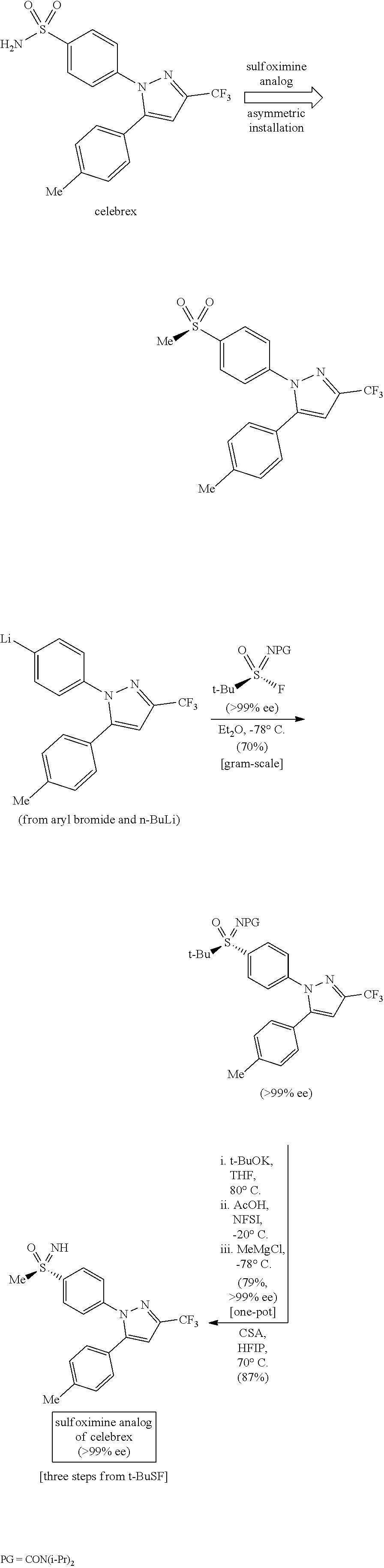

The requisite tert-butyl sulfoximine was prepared as described above using GP-1 on a gram-scale (1.54 g, 2.81 mmol, 70% yield, >99% ee). The one-pot chiral sulfoximine synthesis was employed using GP-12 to prepare urea protected chiral methyl sulfoximine analog of Celebrex (220 mg, 0.431 mmol, 79% yield, >99% ee). Deprotection using GP-13 was followed with no additional changes. Purified by silica gel column chromatography using hexane/EtOAc (0% to 50% EtOAc gradient) to give the product (33 mg, 87 μmol, 87% yield) as a white amorphous solid.

Physical characteristics: White amorphous solid. TLC: R_{f}=0.28 (hexane/EtOAc, 50% EtOAc). ¹H NMR: (500 MHz, CDCl₃) δ 7.99 (d, J=8.7 Hz, 2H), 7.50 (d, J=8.7 Hz, 2H), 7.17 (d, J=7.9 Hz, 2H), 7.11 (d, J=8.3 Hz, 2H), 6.74 (s, 1H), 3.14 (s, 3H), 3.02 (s, 1H), 2.37 (s, 3H) ppm. ¹³C NMR: (126 MHz, CDCl₃) δ 145.37, 144.26 (q, J=38.5 Hz), 143.19, 142.40, 139.94, 129.88, 128.95, 128.82, 125.76, 125.73, 121.11 (q, J=269.2 Hz), 106.53 (d, J=2.1 Hz), 46.19, 21.44 ppm. 19F NMR: (471 MHz, CDCl₃) δ −65.50 ppm. Specific rotation:

$$[ α ] \frac{23}{D} = + 42.33$$

(c 1.00, CHCl₃) HRMS: Calc'd for C₁₈H₁₇N₃OS [M+H⁺] 380.1039; found 380.1039. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 21.587 min, major: 31.287 min.

### Asymmetric Formal Synthesis of Pfizer's PYK2 Inhibitor.

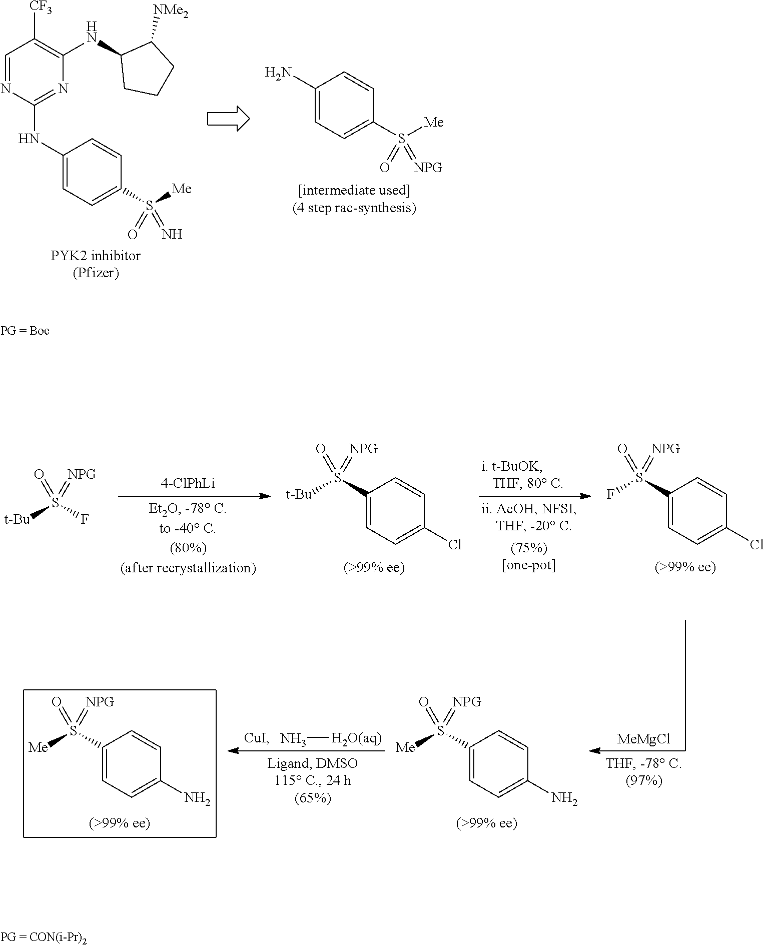

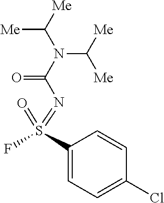 [SMILES: CC(C)N(C(=O)N=[S@@](=O)(F)c1ccc(Cl)cc1)C(C)C]

N,N-diisopropyl urea protected tert-butyl 4-chlorophenyl sulfoximine (552 mg, 1.54 mmol) with >99% ee was obtained by recrystallization of 95% ee material and >90% recovery. GP-14 was applied for the recrystallization using hexanes/EtOAc as a solvent system.

GP-6 was followed for the fluorination with an increased reaction time of step 1 (t-BuOK, THF, 80° C.) from 2 to 3 hours. Purified by silica gel column chromatography using hexane/EtOAc (0% to 20% EtOAc gradient) to give the product (380 mg, 1.18 mmol, 76% yield) as a white amorphous solid.

Physical characteristics: White amorphous solid. TLC: R_{f}=0.43 (hexane/EtOAc, 20% EtOAc). ¹H NMR: (500 MHz, CDCl₃) δ 8.04-7.98 (m, 2H), 7.62-7.56 (m, 2H), 4.15 (s, 1H), 3.83 (s, 1H), 1.32 (dd, J=6.8, 3.8 Hz, 6H), 1.22 (dd, J=6.9, 5.2 Hz, 6H) ppm. 13C NMR: (126 MHz, CDCl₃) § 153.06 (d, J=3.0 Hz), 141.94, 133.94 (d, J=24.1 Hz), 129.97, 129.26, 48.41, 46.02, 21.30, 20.60, 20.55 ppm. 19F NMR: (471 MHz, CDCl₃) δ 69.70 ppm. Specific rotation:

$$[ α ] \frac{23}{D} = + 14 \text{.43}$$

(c 1.00, CHCl₃) HRMS: Calc'd for C₁₃H₁₉ClFN₂O₂S [M+H⁺] 321.0834; found 321.0829. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 12.980 min, major: 15.033 min.

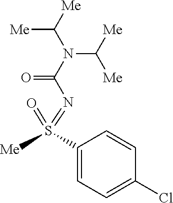 [SMILES: CC(C)N(C(=O)N=[S@@](C)(=O)c1ccc(Cl)cc1)C(C)C]

GP-7 was followed on a larger scale: N,N-diisopropyl urea protected sulfonimidoyl fluoride (321 mg, 1.0 mmol, 1.0 eq) was used with commercially available MeMgCl (0.367 mL, 1.1 eq, 3.0 M in THF, used without titration). Purified by silica gel column chromatography using hexane/EtOAc (0% to 50% EtOAc gradient) to give the product (310 mg, 0.978 mmol, 97% yield) as a white amorphous solid.

Physical characteristics: White amorphous solid. TLC: R_{f}=0.15 (hexane/EtOAc, 30% EtOAc). ¹H NMR: (500 MHZ, CDCl₃) δ 7.91-7.87 (m, 2H), 7.56-7.52 (m, 2H), 4.04 (s, 2H), 3.31 (s, 3H), 1.24 (dd, J=6.9, 2.7 Hz, 12H) ppm. ¹³C NMR: (126 MHz, CDCl₃) δ 158.92, 139.94, 139.22, 129.89, 128.86, 45.11, 21.20 ppm. Specific rotation:

$$[ α ] \frac{23}{D} = - 13.14$$

(c 1.00, CHCl₃) HRMS: Calc'd for C₁₄H₂₂ClN₂O₂S [M+H⁺] 317.1805; found 317.1801. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 14.447 min, major: 26.527 min.

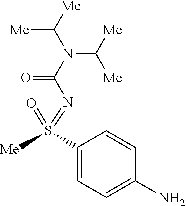 [SMILES: CC(C)N(C(=O)N=[S@@](C)(=O)c1ccc(N)cc1)C(C)C]

Used modified conditions from the reported method (Org. Lett. 2015, 17, 5934-5937). To a flame dried sealed tube with magnetic stir bar, added N,N-diisopropyl urea protected 4-chlorophenyl methyl sulfoximine (158 mg, 0.5 mmol, 1 eq.), CuI (9.5 mg, 10 mol %), ligand prepared from above reference (16.2 mg, 10 mol %) and K₃PO₄ (117 mg, 1.1 eq). The flask was evacuated and back filled with argon three times then anhydrous DMSO (0.5 mL, 1.0 M) was added to the mixture, followed by aqueous ammonia solution (133 μL, 2.0 eq, 30% w/w). The reaction tube was tightly sealed and heated to 115° C. in oil bath (blast shield was placed in front of the reaction). After 24 hours, the reaction was cooled to room temperature then diluted with EtOAc and brine, extracted with EtOAc (15 mL×3). Combined organic layers were washed with water (10 mL×3) and brine (10 mL×3), dried over Na₂SO₄, filtered and concentrated. Further purified by silica gel column chromatography using hexane/EtOAc (0% to 50% EtOAc gradient) to give the product (97 mg, 0.326 mmol, 65% yield) as a white amorphous solid.

Physical characteristics: White amorphous solid. TLC: R_{f}=0.16 (hexane/EtOAc, 50% EtOAc). ¹H NMR: (500 MHZ, DMSO-d6) δ 7.49 (d, 2H), 6.69-6.61 (m, 2H), 6.05 (s, 2H), 4.18 (s, 1H), 3.91-3.55 (m, 1H), 3.25 (s, 3H), 1.15 (dd, J=7.0, 3.7 Hz, 12H) ppm. 13C NMR: (126 MHz, DMSO-d6) δ 158.48, 153.24, 128.76, 124.23, 112.87, 46.23, 44.69, 43.96, 21.10, 20.82 ppm. Specific rotation:

$$[ α ] \frac{23}{D} = - 21.01$$

(c 1.00, CHCl₃) HRMS: Calc'd for C₁₄H₂₄N₃O₂S [M+H⁺] 298.1584; found 289.1573. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 18.167 min, minor: 31.040 min.

### Asymmetric Formal Synthesis of an Allosteric Dopamine Di Receptor Agonist.

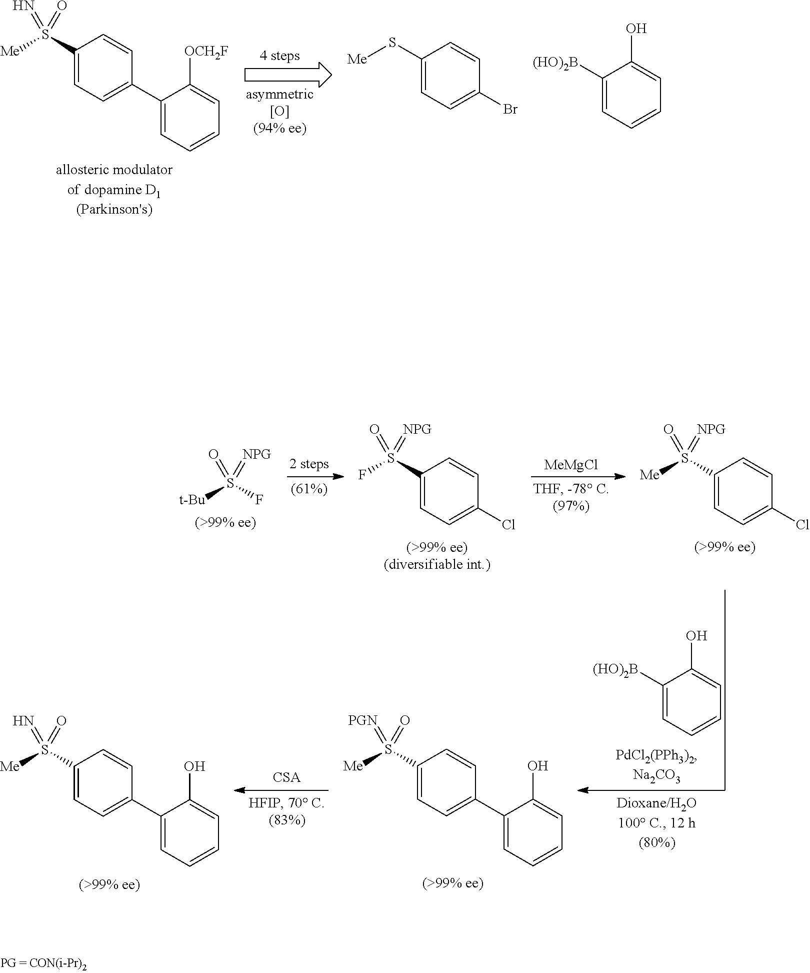

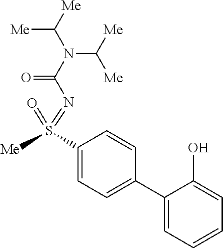 [SMILES: CC(C)N(C(=O)N=[S@@](C)(=O)c1ccc(-c2ccccc2O)cc1)C(C)C]

General Suzuki coupling conditions were used. To a flame dried round bottom flask with magnetic stir bar and under argon, N,N-diisopropyl urea protected 4-Cl-phenyl methyl sulfoximine (95 mg, 0.3 mmol, 1 eq.), commercially available 2-Hydroxyphenylboronic acid (62 mg, 1.5 eq), PdCl₂(PPh₃)₂ (10.5 mg, 10%) and Na₂CO₃ (95.4 mg, 3.0 eq) were added. Then dissolved the mixture with degassed dioxane/H₂O (2:1) solution. Heated to 100° C. and stirred for 12 h in oil bath. Removed for bath and cooled to room temperature. Quenched the reaction with water and extracted with EtOAc (10 mL×3). Washed the combined organic layer with water (10 mL×3) and brine (10 mL×3). Dried over anhydrous Na₂SO₄. Purified by silica gel column chromatography using hexane/EtOAc (0% to 50% EtOAc gradient) to give the product (90 mg, 240 μmol, 80% yield) as a white amorphous solid.

Physical characteristics: White amorphous solid. TLC: R_{f}=0.26 (hexane/EtOAc, 50% EtOAc). ¹H NMR: (500 MHz, CDCl₃) δ 7.95 (d, J=8.5 Hz, 2H), 7.74 (d, J=8.5 Hz, 2H), 7.46 (d, J=4.0 Hz, 1H), 7.29-7.22 (m, 2H), 7.04 (dd, J=8.6, 1.2 Hz, 1H), 6.98 (td, J=7.4, 1.3 Hz, 1H), 4.16 (d, J=68.5 Hz, 2H), 3.34 (s, 3H), 1.34 (dt, J=43.8, 6.6 Hz, 12H) ppm. ¹³C NMR: (126 MHZ, CDCl₃) δ 159.44, 154.04, 144.00, 137.74, 130.40, 130.30, 129.80, 127.14, 126.23, 120.30, 116.99, 46.88, 45.54, 45.36, 21.58, 21.52, 20.87 ppm.

$$[ α ] \frac{23}{D} = - 2 \text{.78}$$

(c 1.00, CHCl₃) HRMS: Calc'd for C₂₀H₂₇N₂O₃S [M+H⁺] Specific rotation: 375.1737; found 375.1721. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 9.480 min, minor: 11.847 min.

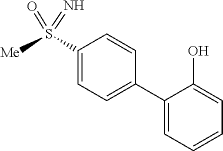 [SMILES: C[S@](=N)(=O)c1ccc(-c2ccccc2O)cc1]

GP-13 was followed and no additional change: N,N-diisopropyl urea protected methyl bi-aryl sulfoximine (0.1 mmol) was used. Purified by silica gel column chromatography using hexane/acetone (0% to 60% acetone gradient) to give the product (20.6 mg, 83 μmol, 83% yield) as a white solid.

Physical characteristics: White amorphous solid. TLC: R_{f}=0.2 (hexane/acetone, 50% acetone). ¹H NMR: (500 MHz, DMSO-d6) δ 9.74 (s, 1H), 7.92 (d, J=8.5 Hz, 2H), 7.74 (d, J=8.5 Hz, 2H), 7.29 (dd, J=7.6, 1.8 Hz, 1H), 7.20 (ddd, J=8.2, 7.3, 1.7 Hz, 1H), 6.96 (dd, J=8.1, 1.2 Hz, 1H), 6.90 (td, J=7.5, 1.2 Hz, 1H), 4.17 (s, 1H), 3.08 (s, 3H) ppm. 13C NMR: (126 MHz, DMSO-d6) δ 154.48, 142.73, 141.96, 130.40, 129.53, 129.47, 126.98, 126.22, 119.60, 116.18, 45.89 ppm. Specific rotation:

$$[ α ] \frac{23}{D} = + 5.53$$

### (c 1.00, MeOH)

HRMS: Calc'd for C₁₃H₁₄NO₂S [M+H⁺] 248.0740; found 248.0740. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: major: 12.407 min, minor: 20.907 min.

### Asymmetric Sulfonimidamide Analog Synthesis of AstraZeneca's Begacestat.

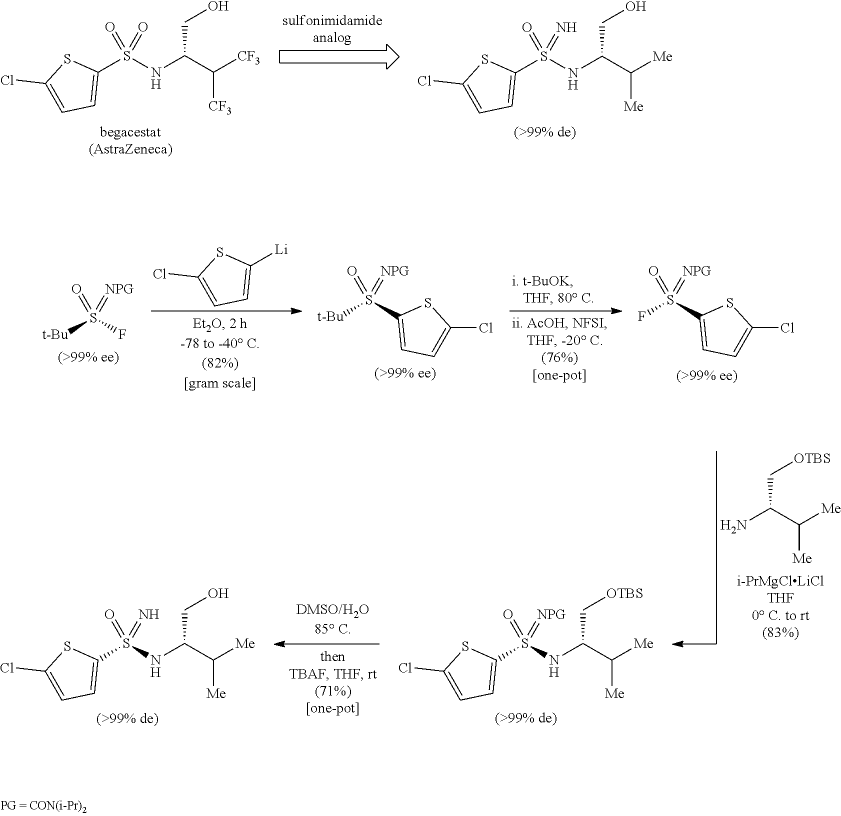

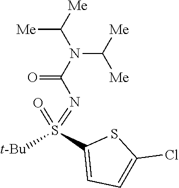 [SMILES: CC(C)N(C(=O)N=[S@](=O)(c1ccc(Cl)s1)C(C)(C)C)C(C)C]

GP-2 was used with no additional change: commercially available 2-bromo-5-chloro-thiophene (721 mg, 6.08 mmol, 1.5 eq.) with t-BuSF (1.08 g, 4.05 mmol, 1 eq.) was used. Purified by silica gel column chromatography using hexane/EtOAc (0% to 45% EtOAc gradient) to give the product (1.21 g, 3.32 mmol, 82% yield, >99% ee) as a white amorphous solid.

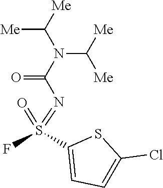 [SMILES: CC(C)N(C(=O)N=[S@](=O)(F)c1ccc(Cl)s1)C(C)C]

GP-6 was followed with no additional change: N,N-diisopropyl urea protected tert-butyl 5-Cl-thiophene sulfoximine (1.2 g, 3.29 mmol, 1.0 eq) was used. Purified by silica gel column chromatography using hexane/EtOAc (0% to 30% EtOAc gradient) to give the product (790 mg, 2.42 mmol, 73% yield) as colorless oil which solidified into a white amorphous solid.

Physical characteristics: White amorphous solid. TLC: R_{f}=0.75 (hexane/EtOAc, 40% EtOAc). ¹H NMR: (500 MHz, CDCl₃) δ 7.78 (d, J=4.2 Hz, 1H), 7.03 (d, J=4.2 Hz, 1H), 4.12 (s, 1H), 3.86 (s, 1H), 1.31 (d, J=6.8 Hz, 6H), 1.23 (dd, J=6.8, 4.2 Hz, 6H) ppm. 13C NMR: (126 MHz, CDCl₃) δ 153.15, 142.04 (d, J=2.1 Hz), 135.33, 131.63 (d, J=30.0 Hz), 127.15, 48.42, 46.05, 21.28, 20.57 ppm. 19F NMR: (471 MHz, CDCl₃) δ 77.11 ppm. Specific rotation:

$$[ α ] \frac{23}{D} = + 7.23$$

(c 1.00, CHCl₃) HRMS: Calc'd for C₁₁H₁₇ClFN₂O₂S [M+H⁺] 327.0399; found 327.0399. Enantiomeric excess: >99% ee. HPLC Conditions: Daicel Chiralpak IC column, 70:30 n-hexane:i-PrOH, flow rate: 1 mL min-1, 25° C., UV detection wavelength: 220 nm, retention time: minor: 9.220 min, major: 10.873 min.

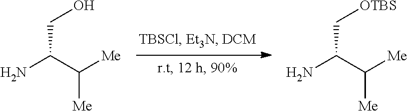 [SMILES: CC(C)[C@H](N)CO ; CC(C)[C@H](N)CO[Si](C)(C)C(C)(C)C]

The protection was finished with reported method with no modification and spectroscopic data was in accordance with the literature. (Org. Lett. 2007, 9, 1, 101-104). ¹H NMR: (500 MHz, CDCl₃) δ 3.65 (dd, J=9.8, 4.1 Hz, 1H), 3.40 (dd, J=9.8, 7.6 Hz, 1H), 2.59 (ddd, J=7.6, 6.2, 4.0 Hz, 1H), 2.14 (s, 2H), 1.72-1.57 (m, 1H), 0.92 (dd, J=7.7, 6.8 Hz, 6H), 0.89 (s, 9H), 0.05 (s, 6H) ppm.

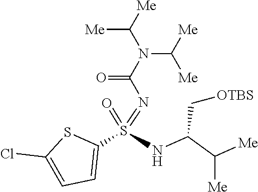 [SMILES: CC(C)[C@@H](CO[Si](C)(C)C(C)(C)C)N[S@@](=O)(=NC(=O)N(C(C)C)C(C)C)c1ccc(Cl)s1]

GP-10 was followed with no additional change: N,N-diisopropyl urea protected 5-Cl-thiophene sulfonimidoyl fluoride (98 mg, 0.3 mmol, 1.0 eq) was used with OTBS protected primary amine (2 eq.). Purified by silica gel column chromatography using hexane/EtOAc (0% to 15% EtOAc gradient) to give the product (130 mg, 248 μmol, 83% yield) as a colorless oil.

Physical characteristics: Colorless oil. TLC: R_{f}=0.55 (hexane/EtOAc, 10% EtOAc). ¹H NMR: (500 MHz, CDCl₃) δ 7.91 (d, J=8.2 Hz, 1H), 7.43 (d, J=4.1 Hz, 1H), 7.39 (d, J=4.0 Hz, 0.01H, from S-diastereomer), 6.84 (d, J=4.1 Hz, 1H), 4.33 (s, 1H), 3.69 (s, 1H), 3.45 (dd, J=10.2, 3.8 Hz, 1H), 3.30 (dd, J=10.2, 5.3 Hz, 1H), 3.16-3.06 (m, 1H), 2.02 (dp, J=13.6, 6.8 Hz, 1H), 1.29 (t, J=8.0 Hz, 6H), 1.13 (dd, J=11.8, 6.8 Hz, 6H), 0.95 (dd, J=7.0, 2.6 Hz, 7H), 0.86 (s, 10H), −0.00 (s, 3H), −0.02 (s, 3H) ppm. ¹³C NMR: (126 MHZ, CDCl₃) δ 157.72, 141.59, 136.80, 130.88, 125.93, 61.42, 59.93, 47.59, 45.19, 29.52, 25.92, 21.21, 21.05, 20.78, 19.12, 18.50, 18.31, −5.52, −5.57 ppm. Specific rotation:

$$[ α ] \frac{23}{D} = - 42. 01$$

(c 1.00, CHCl₃) HRMS: Calc'd for C₂₂H₄₃ClN₃O₃S₂Si [M+H⁺] 524.2198; found 524.2196. Diastereomeric excess: >99% de by ¹HNMR.

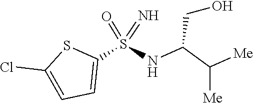 [SMILES: CC(C)[C@@H](CO)N[S@](=N)(=O)c1ccc(Cl)s1]

Modified GP-14 was used to telescope OTBS deprotection: To a 5 mL vial with magnetic stir bar, dissolved N,N-diisopropyl urea protected 5-Cl-thiophene sulfonimidamide (105 mg, 0.2 mmol, 1.0 eq) in DMSO (0.1 M, 2.0 mL) then added water (0.2 mL) and heated to 80° C. for 12 h. Cooled to room temperature then TBAF (1.0 mL, 5.0 eq, 1.0 M in THF, used as received) was added slowly to the mixture. Upon completion (checked by TLC) water (10 mL) was added and extracted with EtOAc (15 mL×3), washed with water (10 mL×3) and brine (10 mL×3), dried over N₂SO₄, filtered and concentrated. Purified by silica gel column chromatography using hexane/acetone (0% to 50% acetone gradient) to give the product (40 mg, 141 μmol, 71% yield) as a colorless oil.

Physical characteristics: Colorless oil. TLC: R_{f}=0.51 (hexane/EtOAc, 50% acetone). ¹H NMR: (500 MHZ, CDCl₃) δ 7.40 (d, J=4.1 Hz, 1H), 6.90 (d, J=4.0 Hz, 1H), 3.72 (m, J=14.3, 12.2, 6.3, 3.7 Hz, 2H), 3.62-3.54 (m, 2H), 3.21 (td, J=6.5, 4.0 Hz, 1H), 3.15 (d, J=4.1 Hz, 0.01 H, from S-diastereomer) 1.77 (dq, J=13.5, 6.8 Hz, 1H), 0.83 (t, J=6.4 Hz, 6H) ppm. ¹³C NMR: (126 MHz, CDCl₃) δ 142.75, 137.13, 131.34, 127.02, 63.66, 62.25, 30.07, 19.34, 18.49 ppm. Specific rotation:

$$[ α ] \frac{23}{D} = + 39. 44$$

(c 1.00, CHCl₃) HRMS: Calc'd for C₉H₁₆N₂O₂S₂ [M+H⁺] 283.0336; found 283.0336. Diastereomeric excess: >99% de by ¹HNMR.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound of Formula II: 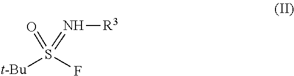
wherein R³ is —C(═O)—N(R⁴)₂, and wherein R⁴ is independently selected at each occurrence from C₃-C₅ alkyl.

2. The compound of claim 1, wherein each R⁴ is isopropyl.

3. The compound of claim 1, wherein the compound is selected from

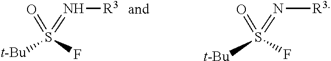

4. The compound of claim 1, wherein the compound is of Formula II-a or Formula II-b

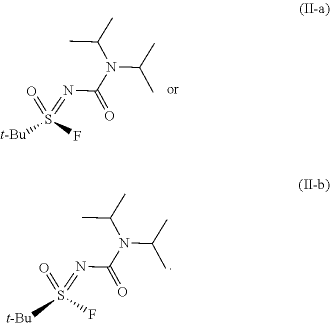 [SMILES: CC(C)N(C(=O)N=[S@](=O)(F)C(C)(C)C)C(C)C ; CC(C)N(C(=O)N=[S@@](=O)(F)C(C)(C)C)C(C)C]

5. (canceled)

6. A process for synthesis of a compound of Formula I 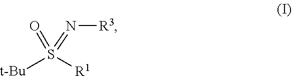
the process comprising:
reacting a compound of Formula II 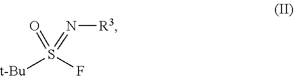
with a compound of Formula III 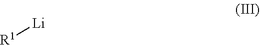
to form the compound of Formula I;
wherein:
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

7. (canceled)

8. The process of claim 6, wherein each R⁴ is isopropyl.

9. The process of claim 8, wherein the compound of Formula II is selected from:

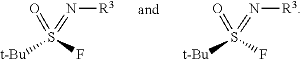

10-13. (canceled)

14. A compound of Formula I prepared according to the process of claim 6.

15. A process for synthesis of a compound of Formula V 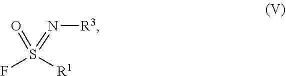
the process comprising:
(a) preparing a compound of Formula I according to the process of claim 6;
(b) reacting the compound of Formula I with a first base to form a compound of Formula IV 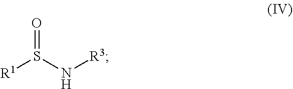
and
(c) reacting the compound of Formula IV with a fluorinating reagent and a second base to form the compound of Formula V 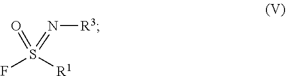
wherein
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

16-29. (canceled)

30. A process for synthesis of a compound of Formula V 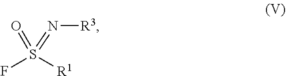
the process comprising:
(a) preparing a compound of Formula I according to the process of claim 6;
(b) reacting the compound of Formula I with a first base to form a reaction mixture;
(c) reacting the first reaction mixture with a fluorinating reagent in the presence of an acid to form the compound of Formula V 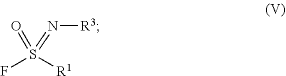
wherein
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

31-42. (canceled)

43. A compound of Formula V prepared according to the process of claim 15.

44. A process for synthesis of a compound of Formula VII 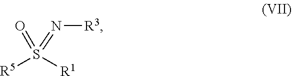
the process comprising:
(a) preparing a compound of Formula V according to the process of claim 15;
(b) reacting the compound of Formula V with a compound of Formula VI 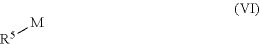
to form the compound of Formula VII;
wherein:
R⁵ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, and (R^{x}R^{y}N)—(C₀-C₃ alkyl)-, wherein each R⁵ is optionally substituted with one or more Z groups as allowed by valency;
M is selected from Li, Na, MgX¹, and MgX¹LiX², wherein X¹ and X² are each independently halo;
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

45-52. (canceled)

53. A process for synthesis of a compound of Formula VII-a 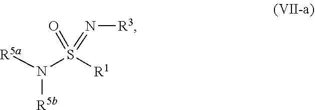
the process comprising:
(a) preparing a compound of Formula V according to the process of claim 15;
(b) reacting the compound of Formula V with a compound of Formula VI-a in the presence of an amine base 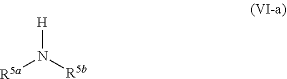
to form the compound of Formula VII;
wherein:
R^{5a} is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5a} is optionally substituted with one or more Z groups as allowed by valency;
R^{5b} is selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5b} is optionally substituted with one or more Z groups as allowed by valency;
or R^{5a} and R^{5b} are brought together with the nitrogen to which they are attached to form a 3- to 8-membered monocyclic or bicyclic heterocycle which is optionally substituted with one or more Z groups as allowed by valency;
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
R³ is —C(═O)—N(R⁴) 2, wherein R⁴ is independently selected at each occurrence from C₃-C₅ alkyl;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

54-58. (canceled)

59. A compound of Formula VII prepared by the process of claim 44.

60. A compound of Formula VII-a prepared by the process of of claim 53.

61. A process of synthesis of a compound of Formula VIII 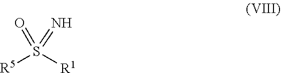
the process comprising:
(a) preparing a compound of Formula VII according to the process of claim 44; and
(b) reacting the compound of Formula VIII in the presence of an acid or by heating to form the compound of Formula VIII;
wherein
R⁵ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, and (R^{x}R^{y}N)—(C₀-C₃ alkyl)-, wherein each R⁵ is optionally substituted with one or more Z groups as allowed by valency;
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

62. A process of synthesis of a compound of Formula VIII-a 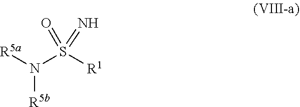
(a) preparing a compound of Formula VII according to the process of claim 53; and
(b) reacting the compound of Formula VIII in the presence of an acid or by heating to form the compound of Formula VIII;
wherein
R^{5a} is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5a} is optionally substituted with one or more Z groups as allowed by valency;
R^{5b} is selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, or (5- to 10-membered monocyclic or bicyclic heteroaryl)-(C₀-C₃ alkyl)-, wherein R^{5b} is optionally substituted with one or more Z groups as allowed by valency;
or R^{5a} and R^{5b} are brought together with the nitrogen to which they are attached to form a 3- to 8-membered monocyclic or bicyclic heterocycle which is optionally substituted with one or more Z groups as allowed by valency;
R¹ is selected from C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, and (5- to 10-membered monocyclic or bicyclic heteroaryl)-(C₀-C₃ alkyl)-, wherein each R¹ is optionally substituted with one or more Z groups as allowed by valency;
Z is independently selected at each occurrence from halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-, wherein each Z is optionally substituted with one or more Y groups as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which is optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which is optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

63-66. (canceled)

67. A compound of Formula VIII or Formula VIII-a prepared by the process of claim 61.
